(19) United States
(12) Patent Application Publication
(10) Pub. No.: US 2004/0063658 A1
(43) Pub. Date: Apr. 1, 2004

(54) Nucleoside derivatives for treating hepatitis C virus infection

(76) Inventors: Christopher Don Roberts, Belmont, CA (US); Natalia B. Dyatkina, Mountain View, CA (US); Jesse D. Keicher, Menlo Park, CA (US); Sebastian Johannes Reinhard Liehr, East Palo Alto, CA (US); Eric Jason Hanson, San Francisco, CA (US)

Correspondence Address: BURNS, DOANE, SWECKER & MATHIS, L.L.P., P.O. Box 1404, Alexandria, VA, 22313-1404, US

(21) Appl. No.: 10/431,631

(22) Filed: May 6, 2003

Related U.S. Application Data
(60) Provisional application No. 60/378,624, filed on May 6, 2002.
(60) Provisional application No. 60/392,871, filed on Jun. 28, 2002.

(51) Int. Cl. A61K 31/7076; A61K 31/7072; C07H 19/16; C07H 19/06

(52) U.S. Cl. 514/45; 514/50; 536/27.21; 536/28.53

(57) ABSTRACT

Disclosed are compounds, compositions and methods for treating hepatitis C virus infections.

### CROSS-REFERENCE TO RELATED APPLICATIONS

This application claims the benefit of U.S. Provisional Application Serial No. 60/378,624, filed on May 6, 2002 and U.S. Provisional Application Serial No. 60/392,871, filed on Jun. 28, 2002, the disclosures of which are incorporated herein in their entirety.

### FIELD OF THE INVENTION

The invention relates to the field of pharmaceutical chemistry, in particular to compounds, compositions and methods for treating hepatitis C virus infections.

### REFERENCES

The following publications and patents are cited in this application as superscript numbers:

1. Chen, et al., Med. Assoc., 95(1):6-12 (1996)

2. Cornberg, et al., “Hepatitis C: therapeutic perspectives.” Forum (Genova), 11(2):154-62 (2001)

3. Dymock, et al., Antivir. Chem. Chemother. 11(2):79-96 (2000)

4. Devos, et al., International Patent Application Publication No. WO 02/18404 A2, published Mar. 7, 2002

5. Sommadossi, et al., International Patent Application Publication No. WO 01/90121, published May 23, 2001

6. Ducrocq, C.; et al., Tetrahedron, 32:773 (1976).

7. Rizkalla, B. H.; Broom, A. D., J. Org. Chem., 37(25):3980 (1972).

8. Anderson, G. L.; Broom, A. D., J. Org. Chem., 42(6):997 (1977).

9. Rizkalla, B. H.; Broom, A. D., J. Org. Chem., 37(25):3975 (1972).

10. Furukawa, Y.; Honjo, M., Chem. Pharm. Bull, 16(6):1076 (1968).

11. Ektova, L. V.; et al., Bioorg. Khim., 5:1369 (1979).

12. De Clercq, E.; et al., J. Med. Chem., 26(5):661 (1983).

13. Robins, M. J.; Barr, P. J., J. Org. Chem., 48(11):1854 (1983).

14. Griengl, H., J. Med. Chem., 28(11):1679 (1985).

15. Lichtenhaler, F. W.; Cuny, E., Chem. Ber., 114:1610 (1981).

16. Hamilton, H. W.; Bristol, J. A., J. Med. Chem., 26(11):1601 (1983).

17. Seela, F.; Steker, H., Liebigs Ann. Chem., p. 1576 (1983).

18. Winkley, M. W.; et al., J. Heterocycl. Chem., 8:237 (1971).

19. Barascut, J. L.; et al., J. Carbohydr. Nucleosides Nucleotides, 3(5&6):281 (1976).

20. Kiriasis, L.; Pfleiderer, W., Nucleosides Nucleotides, 8(7):1345 (1989).

21. Schneider, H. -J.; Pfleiderer, W., Chem. Berich., 107:3377 (1974).

22. Angew. Chem. Int. Ed. Engl., 35:1968 (1996)

23. Hildbrand, S.; et al., Helv. Chim. Acta, 79:702 (1996).

24. De Las Heras, F.; et al., J. Heterocycl. Chem., 13:175 (1976).

25. Tam, S. Y -K.; et al., J. Heterocycl. Chem., 13:1305 (1976).

26. Chu, C. K.; et al., J. Heterocycl. Chem., 17:1435 (1980).

27. De Bernardo, S.; Weigele, M., J. Org. Chem., 42(1):109 (1977).

28. Saureamid-Reaktionen, L.; Orthoamide, I., Chem. Ber., 101:41 (1968).

29. Lim, M. -I.; Klein, R. S.; Fox, J. J., Tet. Lett., 21:1013 (1981).

30. Yamazaki, A.; et al., J. Org. Chem., 32:1825 (1967).

31. Yamazaki, A.; Okutsu, M., J. Heterocycl. Chem., 1978,15:353 (1978)

32. Lim, M. -I.; Klein, R. S., Tet. Lett., 22:25 (1981).

33. Bhattacharya, B. K.; et al., Tet. Lett., 27(7):815 (1986).

34. Grisis, N. S.; et al., J. Med. Chem., 33:2750 (1990).

35. Li, N-. S.; Tang, X. -Q.; Piccirilli, J. A., Organic Letters, 3(7):1025 (2001).

36. Cristalli, G.; et al., J. Med. Chem., 30(9):1686 (1987).

37. Seela, F.; et al., Nucleosides Nucleotides, 17(4):729 (1998).

38. Sagi, G.; et al., J. Med. Chem. 35(24):4549 (1992).

39. Hawkins, M. E.; et al., Nucleic Acids Research, 23(15):2872 (1995).

40. Mandal, S. B., et al., Synth. Commun., 9:1239 (1993).

41. Witty, D. R., et al., Tet. Lett., 31: 4787 (1990).

42. Ning, J. et al., Carbohydr. Res., 330:165 (2001).

43. Yokoyama, M., et al., J. Chem. Soc. Perkin Trans. I, 2145 (1996).

44. Carroll, S. S., et al., ., International Patent Application Publication No. WO 02/057287, published Jul. 25, 2002

45. Carroll, S. S., et al., ., International Patent Application Publication No. WO 02/057425, published Jul. 25, 2002

All of the above publications, patents and patent applications are herein incorporated by reference in their entirety to the same extent as if each individual publication, patent or patent application was specifically and individually indicated to be incorporated by reference in its entirety.

### STATE OF THE ART

Hepatitis C virus (HCV) causes a liver damaging infection that can lead to cirrhosis, liver failure or liver cancer, and eventually death. HCV is an enveloped virus containing a positive-sense single-stranded RNA genome of approximately 9.4 kb, and has a virion size of 30-60 nm.¹

HCV is a major causative agent for post-transfusion and for sporadic non-A, non-B hepatitis. Infection by HCV is insidious in a high proportion of chronically infected (and infectious) carriers who may not experience clinical symptoms for many years.

HCV is difficult to treat and it is estimated that there are 500 million people infected with it worldwide. No effective immunization is currently available, and hepatitis C can only be controlled by other preventive measures such as improvement in hygiene and sanitary conditions and interrupting the route of transmission.

At present, the only acceptable treatment for chronic hepatitis C is interferon (IFN-alpha) and this requires at least six (6) months of treatment and/or ribavarin, which can inhibit viral replication in infected cells and also improve liver function in some people.

IFN-alpha belongs to a family of naturally occurring small proteins with characteristic biological effects such as antiviral, immunoregulatory and antitumoral activities which are produced and secreted by most animal nucleated cells in response to several diseases, in particular viral infections. IFN-alpha is an important regulator of growth and differentiation affecting cellular communication and immunological control. Treatment of HCV with interferon, however, has limited long term efficacy with a response rate about 25%. In addition, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction.

Ribavirin (1-β-D-ribofuranosyl-1H-1,2,-4-triazole-3-carboxamide), an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of PEG-IFN plus ribavirin. However, a number of patients still have significant side effects, primarily related to ribaviran. Ribavirin causes significant hemolysis in 10-20% of patients treated at currently recommended doses, and the drug is both teratogenic and embryotoxic.

Other approaches are being taken to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. NS3/4A serine protease, ribonucleic acid (RNA) helicase, RNA-dependent RNA polymerase are considered as potential targets for new drugs.^{2,3}

Devos, et al.⁴ describes purine and pyrimidine nucleoside derivatives and their use as inhibitors of HCV RNA replication. Sommadossi, et al.⁵ describes 1′, 2+ or 3′-modified nucleosides and their use for treating a host infected with HCV. Carroll, et al.^{44,45}, describe nucleosides as inhibitors of RNA-dependent RNA viral polymerase.

Given the fact of the worldwide epidemic level of HCV, there is a strong need for new effective drugs for HCV treatment. The present invention provides nucleoside derivatives for treating HCV infections.

### SUMMARY OF THE INVENTION

This invention is directed to novel compounds that are useful in the treatment of HCV in mammals. Specifically, the compounds of this invention are represented by formula Ia, Ib and Ic below:

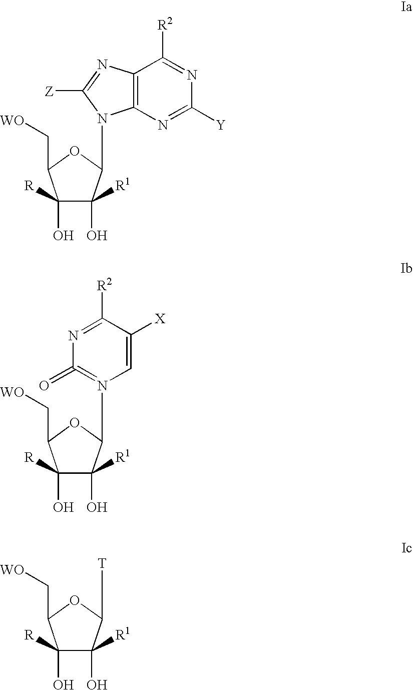

wherein R and R¹ are independently selected from the group consisting of:

hydrogen,

alkyl,

substituted alkyl,

alkenyl,

substituted alkenyl,

alkynyl, and

substituted alkynyl

provided that R and R¹ are not both hydrogen;

R² is selected from the group consisting of: alkyl,

substituted alkyl,

cycloalkyl,

substituted cycloalkyl,

alkenyl,

substituted alkenyl,

alkynyl,

substituted alkynyl,

acylamino

guanidino

amidino

thioacylamino,

hydroxy,

alkoxy,

substituted alkoxy,

halo,

nitro,

thioalkyl

aryl,

substituted aryl,

heteroaryl,

substituted heteroaryl,

—NR³R⁴ where R³ and R⁴ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic and where R³ and R⁴ are joined to form, together with the nitrogen atom bond thereto, a heterocyclic, substituted heterocyclic, heteroaryl, or substituted heteroaryl,

—NR⁵NR³R⁴ where R³ and R⁴ are as defined above and R⁵ is selected from the group consisting of hydrogen and alkyl,

W is selected from the group consisting of:

hydrogen,

phosphate (including monophosphate, diphosphate, triphosphate or a stablilized phosphate prodrug),

phosphonate,

acyl,

alkyl,

sulfonate ester selected from the group consisting of alkyl esters, substituted alkyl esters, alkenyl esters, substituted alkenyl esters, aryl esters, substituted aryl esters, heteroaryl esters, substituted heteroaryl esters, heterocyclic esters and substituted heterocyclic esters,

a lipid,

an amino acid,

a carbohydrate,

a peptide, and

cholesterol;

X is selected from the group consisting of:

hydrogen,

halo,

alkyl,

substituted alkyl, and

—NR³R⁴ where R³ and R⁴ are as identified above;

Y is selected from the group consisting of:

hydrogen,

halo,

hydroxy,

alkylthio

—NR³R⁴ where R³ and R⁴ are as identified above;

Z is selected from the group consisting of:

hydrogen,

halo,

hydroxy,

alkyl,

azido, and

—NR³R⁴ where R³ and R⁴ are as identified above

—NR⁵NR³R⁴ where R³, R⁴ and R⁵ are as identified above;

and wherein T is selected from the group consisting of

a) 1- and 3-deazapurines of the formula below:

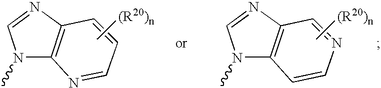

b) purine nucleosides of the formula below:

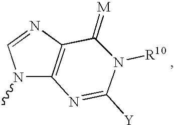

c) benzimidazole nucleosides of the formula below:

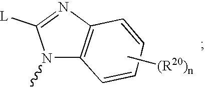

d) 5-pyrrolopyridine nucleosides of the formula below:

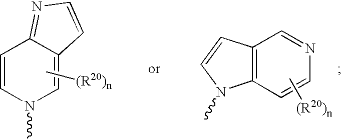

e) 4-pyrimidopyridone sangivamycin analogs of the formula below:

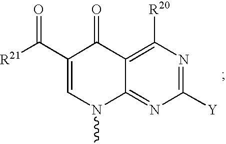

f) 2-pyrimidopyridone sangivamycin analogs of the formula below:

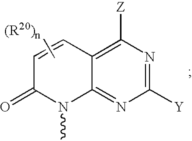

g) 4-pyrimidopyridone sangivamycin analogs of the formula below:

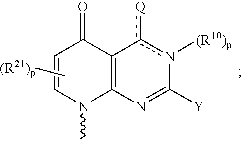

h) pyrimidopyridine analogs of the formulae below:

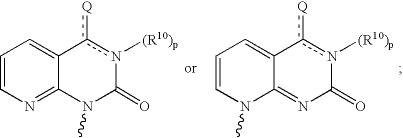

i) pyrimido-tetrahydropyridines of the formula below:

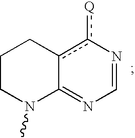

j) Furanopyrimidines (& tetrahydro furanopyrimidines) of the formulae below:

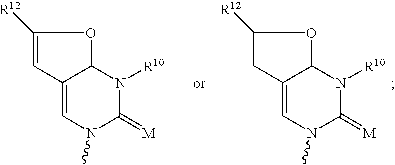

k) pyrazolopyrimidines of the formula below:

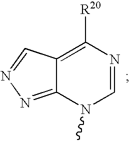

l) pyrolopyrimidines of the formula below:

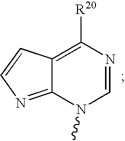

m) triazolopyrimidines of the formula below:

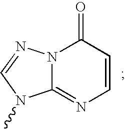

n) pteridines of the formula below:

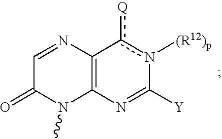

o) pyridine C-nucleosides of the formula below:

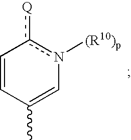

p) pyrazolotriazine C-nucleosides of the formula below:

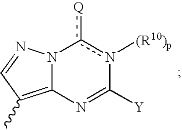

q) Indole nucleosides of the formula below:

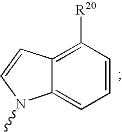

r) a base of the formula below:

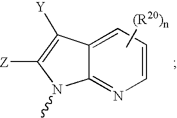

s) a base of the formula below:

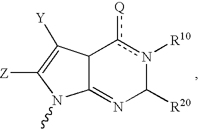

t) a base of the formula below:

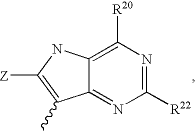

u) a base of the formula below:

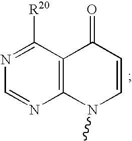

v) a base of the formula below:

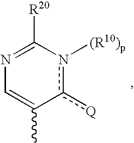

w) a base of the formula below:

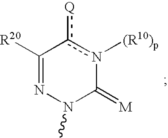

x) a base of the formula below:

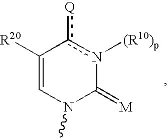

y) a base of the formula below:

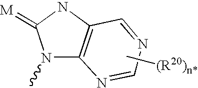

and further wherein one of bonds characterized by [symbol] is a double bond and the other is a single bond provided that, when the [symbol] between the N and a ring carbon is a double bond, then p is 0 and when the [symbol] between Q and a ring carbon is a double bond, then p is 1;

each p is independently 0 or 1;

each n is independently 0 or an integer from 1 to 4;

each n* is independently 0 or an integer from 1 to 2;

L is selected from the group consisting of hydrogen, halo, alkyl, substituted alkyl, amino, substituted amino, azido, and nitro;

Q is selected from the group consisting of hydrogen, halo, ═O, —OR¹¹, ═N—R¹¹, —NHR¹¹, ═S, —SR¹¹, aryl, substituted aryl, heteroaryl, substituted heteroaryl, heterocyclic, and substituted heterocyclic;

M is selected from the group consisting of ═O, ═N—R¹¹, and ═S;

Y is as defined above;

R¹⁰ is selected from the group consisting of hydrogen, alkyl, substituted alkyl, cycloalkyl, substituted cycloalkyl, heterocyclic, substituted heterocyclic, alkylthioether, substituted alkylthioether, aryl, substituted aryl, heteroaryl, and substituted heteroaryl, with the proviso that when T is b), s), v), w) or x), then R¹⁰ is not hydrogen;

each R¹¹ and R¹² is independently selected from the group consisting of hydrogen, alkyl, substituted alkyl, cycloalkyl, substituted cycloalkyl, heterocyclic, substituted heterocyclic, amino, substituted amino, alkylthioether, substituted alkylthioether, aryl, substituted aryl, heteroaryl, and substituted heteroaryl;

each R²⁰ is independently selected from the group consisting of:

hydrogen,

alkyl,

substituted alkyl,

aryl,

substituted aryl,

cycloalkyl,

substituted cycloalkyl,

alkenyl,

substituted alkenyl,

alkynyl,

substituted alkynyl,

heteroaryl,

substituted heteroaryl,

acylamino

guanidino

amidino

thioacylamino,

alkoxy,

substituted alkoxy,

alkylthio,

nitro,

halo,

hydroxy

—NR³R⁴ where R³ and R⁴ are as defined above,

—NR⁵NR³R⁴ where R³, R⁴ and R⁵ are as defined above;

each R²¹ and R²² are independently selected from the group consisting of:

—NR³R⁴ where R³ and R⁴ are as defined above, and

—NR⁵NR³R⁴ where R³, R⁴ and R⁵ are as defined above

—C(O)NR³R⁴ where R³ and R⁴ are as defined above, and

—C(O)NR⁵NR³R⁴ where R³, R⁴ and R⁵ are as defined above;

and pharmaceutically acceptable salts thereof;

with the provisos that

1) for a compound of formula Ia, when Z is Z is hydrogen, halo, hydroxy, azido, or NR³R⁴, where R³ and R⁴ are independently H, or alkyl; Y is hydrogen or —NR³R⁴ where R³ and R⁴ are independently hydrogen or alkyl; then R² is not alkyl, alkoxy, halo, hydroxy, CF₃, or —NR³R⁴ where R³ and R⁴ are independently hydrogen or alkyl;

2) for a compound of formula la, when Z is hydrogen, halo, hydroxy, azido, or NR³R⁴, where R³ and R⁴ are independently H, or alkyl; Y is hydrogen, halo, hydroxy, or alkylthio; then R² is not

alkyl,

substituted alkyl, wherein the substituted alkyl is substituted with hydroxyl, amino, alkylamino, arylamino, alkoxy, aryloxy, nitro, cyano, sulfonic acid, sulfate, phosphonic acid, phosphate, or phosphonate, either unprotected or protected,

halo,

hydroxy,

alkoxy,

thioalkyl, or

—NR³R⁴, where R³ and R⁴ are independently hydrogen, alkyl or alkyl substituted with hydroxyl, amino, alkylamino, arylamino, alkoxy, aryloxy, nitro, cyano, sulfonic acid, sulfate, phosphonic acid, phosphate, or phosphonate, either unprotected or protected);

3) for a compound of formula Ib, when X is hydrogen, halo, alkyl, CF₃ or —NR³R⁴ where R³ is hydrogen and R⁴ is alkyl, then R² is not alkyl, alkoxy, halo, hydroxy, CF₃, or —NR³R⁴ where R³ and R⁴ are independently hydrogen or alkyl;and

4) for a compound of formula Ib, R² is not, halo, alkoxy, hydroxy, thioalkyl, or —NR³R⁴ (where R³ and R⁴ are independently hydrogen, alkyl or alkyl substituted with hydroxyl, amino, alkylamino, arylamino, alkoxy, aryloxy, nitro, cyano, sulfonic acid, sulfate, phosphonic acid, phosphate, or phosphonate, either unprotected or protected)

And further provided that the compound of Formual Ia, Ib or Ic is not

a) 2-Hydroxymethyl-5-(6-phenyl-purin-9-yl)-tetrahydro-furan-3,4-diol; or

b) b) 2-Hydroxymethyl-5-(6-thiophen-3-yl-purin-9-yl)-tetrahydro-furan-3,4-diol.

In a preferred embodiment R¹ is selected from the group consisting of —CH₃, —CF₃, —CH═CH₂, and —C CH, more preferrably CH₃.

In another preferred embodiment when T is a base of formula a) then T is a 3-deazapurine.

This invention is further directed to a compound of Formula II:

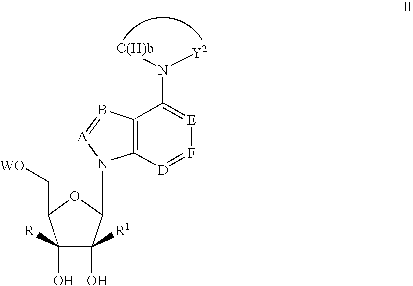

wherein R and R¹ are independently selected from the group consisting of:

hydrogen,

alkyl,

substituted alkyl,

alkenyl,

substituted alkenyl,

alkynyl,

substituted alkynyl,

halogen,

azido,

amino, and

substituted amino;

provided that R and R¹ are not both hydrogen;

Y² is CH₂, N, S, SO, or SO₂;

N together with —C(H)_{b} and Y² forms a heterocyclic, substituted heterocyclic, heteroaryl or substituted heteroaryl group wherein each of said heterocyclic, substituted heterocyclic, heteroaryl or substituted heteroaryl group is optionally fused to form a bi- or multi-fused ring system (preferably no more than 5 fused rings) with one or more ring structures selected from the group consisting of cycloalkyl, cycloalkenyl, heterocyclic, aryl and heteroaryl group which, in turn, each of such ring structures is optionally substituted with 1 to 4 substituents selected from the group consisting of hydroxyl, halo, alkoxy, substituted alkoxy, thioalkyl, substituted thioalkyl, aryl, heteroaryl, heterocyclic, nitro, cyano, carboxyl, carboxyl esters, alkyl, substituted alkyl, alkenyl, substituted alkenyl, alkynyl, substituted alkynyl, amino, and substituted amino;

b is an integer equal to 0 or 1;

A, B, D, and E are independently selected from the group consisting of >N, >CH, >C—CN, >C—NO₂, >C-alkyl, >C-substituted alkyl, >C—NHCONH₂, >C—CONR¹⁵R¹⁶, >C—COOR¹⁵, >C-hydroxy, >C-alkoxy, >C-amino, >C-alkylamino, >C-dialkylamino, >C-halogen, >C-(1,3-oxazol-2-yl), >C-(1,3-thiazol-2-yl) and >C-(imidazol-2-yl);

F is selected from >N, >C—CN, >C—NO₂, >C-alkyl, >C-substituted alkyl, >C—NHCONH₂, >C—CONR¹⁵R¹⁶, >C—COOR⁵, >C-alkoxy, >C-(1,3-oxazol-2-yl), >C-(1,3-thiazol-2-yl), >C-(imidazol-2-yl), and >C—Y, where Y is selected from the group consisting of hydrogen, halo, hydroxy, alkylthioether, and —NR³R⁴ where R³ and R⁴ are independently selected from the group consisting of hydrogen, hydroxy, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclic, substituted heterocyclic and where R³ and R⁴ are joined to form, together with the nitrogen atom bond thereto, a heterocyclic group, provided that only one of R³ and R⁴ are hydroxy, alkoxy, or substituted alkoxy;

R¹⁵ and R¹⁶ are independently selected from the group consisting of:

hydrogen,

alkyl,

substituted alkyl,

cycloalkyl,

substituted cycloalkyl,

aryl,

substituted aryl,

heteroaryl,

substituted heteroaryl, and

R¹⁵ and R¹⁶ together with the atom to which they are attached may form a cycloalkyl, substituted cycloalkyl, hetercycloalkyl, substituted heterocylcoalkyl, heteroaryl, or substituted heteroaryl;

W is selected from the group consisting of:

hydrogen,

phosphate (including monophosphate, diphosphate, triphosphate or a stablilized phosphate prodrug),

phosphonate,

acyl,

alkyl,

sulfonate ester selected from the group consisting of alkyl esters, substituted alkyl esters, alkenyl esters, substituted alkenyl esters, aryl esters, substituted aryl esters, heteroaryl esters, substituted beteroaryl esters, heterocyclic esters and substituted heterocyclic esters,

a lipid,

an amino acid,

a carbohydrate,

a peptide, and

cholesterol;

and pharmaceutically acceptable salts thereof.

In a preferred embodiment, the compounds of formula II are represented by formula IIA:

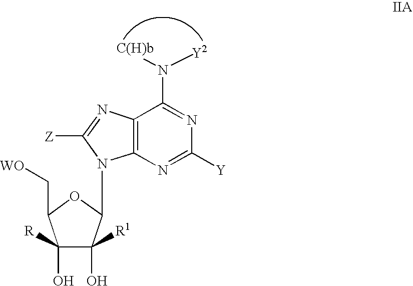

wherein R and R¹ are independently selected from the group consisting of:

hydrogen,

alkyl,

substituted alkyl,

alkenyl,

substituted alkenyl,

alkynyl,

substituted alkynyl,

halogen,

azido,

amino, and

substituted amino;

provided that R and R¹ are not both hydrogen;

Y²is CH₂, N, S, SO, or SO₂;

N together with —C(H)_{b} and Y² forms a heterocyclic, substituted heterocyclic, heteroaryl or substituted heteroaryl group wherein each of said heterocyclic, substituted heterocyclic, heteroaryl or substituted heteroaryl group is optionally fused to form a bi- or multi-fused ring system (preferably no more than 5 fused rings) with one or more ring structures selected from the group consisting of cycloalkyl, cycloalkenyl, heterocyclic, aryl and heteroaryl group which, in turn, each of such ring structures is optionally substituted with 1 to 4 substituents selected from the group consisting of hydroxyl, halo, alkoxy, substituted alkoxy, thioalkyl, substituted thioalkyl, aryl, heteroaryl, heterocyclic, nitro, cyano, carboxyl, carboxyl esters, alkyl, substituted alkyl, alkenyl, substituted alkenyl, alkynyl, substituted alkynyl, amino, and substituted amino;

b is an integer equal to 0 or 1;

W is selected from the group consisting of:

hydrogen,

phosphate (including monophosphate, diphosphate, triphosphate or a stablilized phosphate prodrug),

phosphonate,

acyl,

alkyl,

sulfonate ester selected from the group consisting of alkyl esters, substituted alkyl esters, alkenyl esters, substituted alkenyl esters, aryl esters, substituted aryl esters, heteroaryl esters, substituted heteroaryl esters, heterocyclic esters and substituted heterocyclic esters,

a lipid,

an amino acid,

a carbohydrate,

a peptide, and

cholesterol;

Y is selected from the group consisting of Y is selected from the group consisting of:

hydrogen,

halo,

hydroxy,

alkylthioether

—NR³R⁴ where R³ and R⁴ are independently selected from the group consisting of hydrogen, hydroxy, alkyl, substituted alkyl, alkenyl, substituted alkenyl, alkynyl and substituted alkynyl, alkoxy, substituted alkoxy, aryl, substituted aryl, heteroaryl, substituted heteroaryl, heterocyclic, substituted heterocyclic and where R³ and R⁴ are joined to form, together with the nitrogen atom bond thereto, a heterocyclic group, provided that only one of R³ and R⁴ are hydroxy, alkoxy, or substituted alkoxy;

Z is selected from the group consisting of:

hydrogen,

halo,

hydroxy,

alkyl,

azido, and

—NR³R⁴ where R³ and R⁴ are independently selected from the group consisting of hydrogen, hydroxy, alkyl, substituted alkyl, alkenyl, substituted alkenyl, alkynyl and substituted alkynyl, alkoxy, substituted alkoxy, aryl, substituted aryl, heteroaryl, substituted heteroaryl, heterocyclic, substituted heterocyclic and where R³ and R⁴ are joined to form, together with the nitrogen atom bond thereto, a heterocyclic group, provided that only one of R³ and R⁴ are hydroxy, alkoxy, or substituted alkoxy;

and pharmaceutically acceptable salts thereof.

Compounds included within the scope of this invention include, for example, those set forth below (including pharmaceutically acceptable salts thereof):

| Cmpd# | Structure | Name |
|---|---|---|
| 1 | 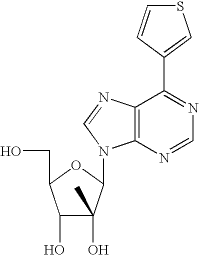 | 9-(2′-C-methyl-β-D-ribofuranosyl)- 6-(thiophen-3-yl)-purine |
| 2 | 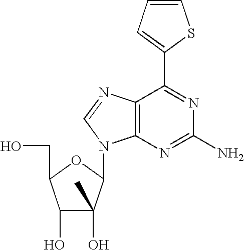 | 9-(2′-C-methyl-β-D-ribofuranosyl)- 6-thiophen-2-yl)-2-aminopurine |
| 3 | 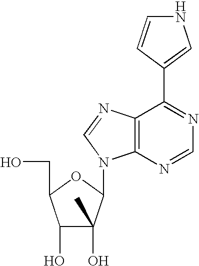 | 9-(2′-C-methyl-β-D-ribofuranosyl)- 6-(prrol-3-yl)-purine |
| 4 | 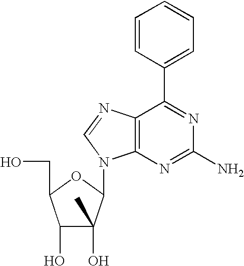 | 9-(2′-C-methyl-β-D-ribofuranosyl)- 6-phenyl-2-aminopurine |
| 5 | 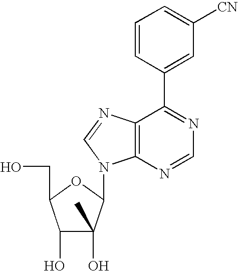 | 9-(2′-C-methyl-β-D-ribofuranosyl)- 6-(3-cyanophen-yl)-purine |
| 6 | 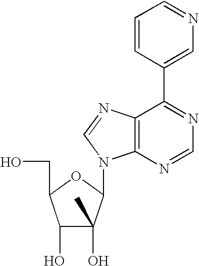 | 9-(2′-C-methyl-β-D-ribofuranosyl)- 6-(pyridin-3-yl)-purine |
| 7 | 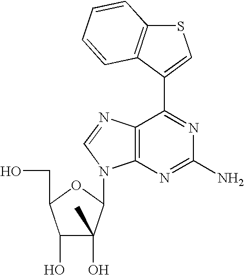 | 9-(2′-C-methyl-β-D-ribofuranosyl)- 6-Benzo[b]thiophen-3-yl)- aminopurine |
| 8 | 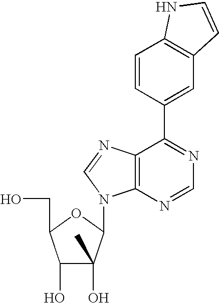 | 9-(2′-C-methyl-β-D-ribofuranosyl)- 6-(1H-Indol-5-yl)-purine |
| 9 | 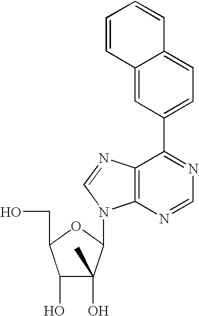 | 9-(2′-C-methyl-β-D-ribofuranosyl)- 6-(naphthalen-2-yl)-purine |
| 10 | 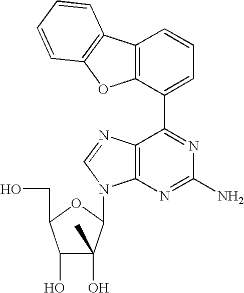 | 9-(2′-C-methyl-β-D-ribofuranosyl)- 6-(dibenzofuran-4-yl)-2- aminopurine |
| 11 | 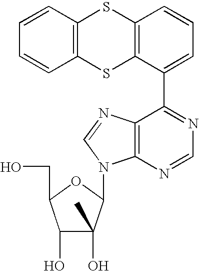 | 9-(2′-C-methyl-β-D-ribofuranosyl)- 6-(thianthren-1-yl)-purine |
| 13 | 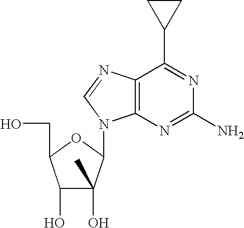 | 9-(2′-C-methyl-β-D-ribofuranosyl)- 6-cyclopropyl-2-aminopurine |
| 14 | 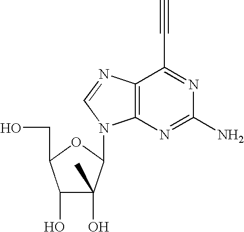 | 9-(2′-C-methyl-β-D-ribofuranosyl)- 6-(ethynyl)-purine |
| 15 | 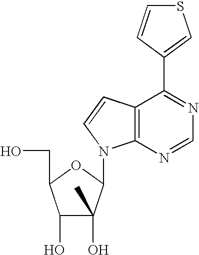 | 7-(2′-C-methyl-β-D-ribofuranosyl)- 4-thiophen-3-yl-7H-pyrrolo[2,3- d]pyrimidine |
| 16 | 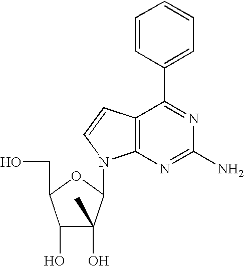 | 7-(2′-C-methyl-β-D-ribofuranosyl)- 4-phenyl-7H-pyrrolo[2,3- d]pyrimidin-2-ylamine |
| 17 | 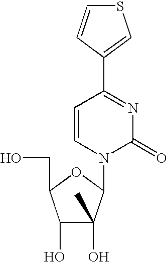 | 1-(2′-C-methyl-β-D-ribofuranosyl)- 4-thiophen-3-yl-1H-pyrimidin-2-one |
| 18 | 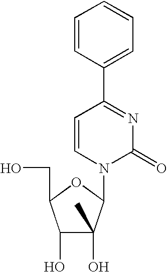 | 1-(2′-C-methyl-β-D-ribofuranosyl)- 4-phenyl-1H-pyrimidin-2-one |
| 19 | 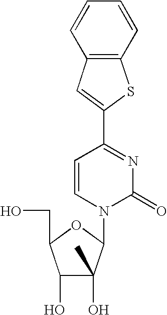 | 1-(2′-C-methyl-β-D-ribofuranosyl)- 4-benzo[b]thiophen-2-yl-1H- pyrimidin-2-one |
| 21 | 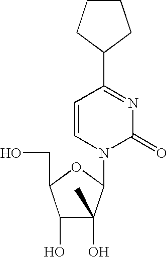 | 1-(2′-C-methyl-β-D-ribofuranosyl)- 4-cyclopentyl-1h-pyrimidin-2-one |
| 22 | 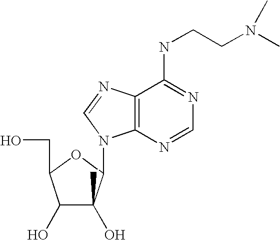 | 9-(2′-C-methyl-β-D-ribofuranosyl)- N⁶-(2-dimethylaminoethyl)-adenine |
| 23 | 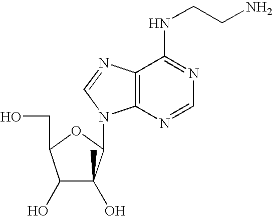 | 9-(2′-C-methyl-β-D-ribofuranosyl)- N⁶-(2-aminoethyl)adenine |
| 24 | 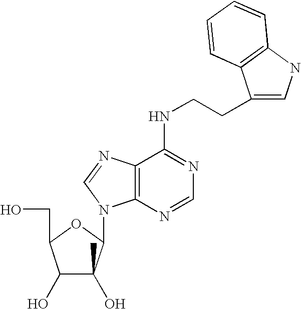 | 9-(2′-C-methyl-β-D-ribofuranosyl)- N⁶-[2-(3H-indol-3-yl)- ethyl]adenine |
| 25 | 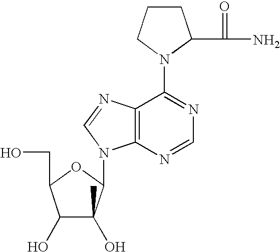 | 9-(2′-C-methyl-β-D-ribofuranosyl)- 6-[2-aminocarbonyl-(pyrrolidine-1- yl)]-purine |
| 26 | 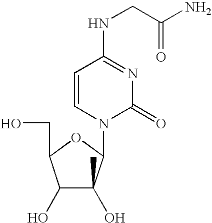 | 1-(2′-C-methyl-β-D-ribofuranosyl)- N⁴-(aminocarbonylmethyl)cytidine |
| 27 | 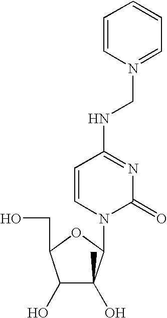 | 9-(2′-C-methyl-β-D-ribofuranosyl)- N⁴-[(pyridin-1-yl)-methyl]cytidine |
| 30 | 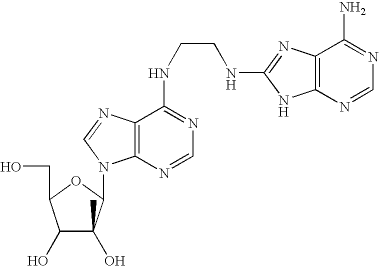 | 1-(2′-C-methyl-β-D-ribofuranosyl)- N⁶-[ (adenin-8-yl)-aminoethy]adenine |
| 31 | 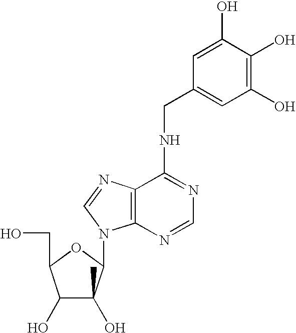 | 9-(2′-C-methyl-β-D-ribofuranosyl)- N⁶-[(benzene-3,4,5- triol)methyl]adenine |
| 32 | 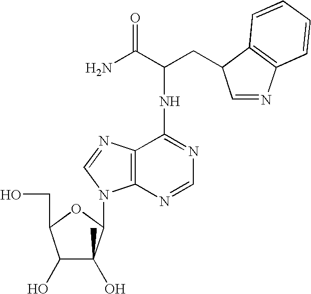 | 9-(2′-C-methyl-β-D-ribofuranosyl)- N⁶-[1-aminocarbonyl-2-(3H-indol- 3-yl)-ethyl]adenine |
| 33 | 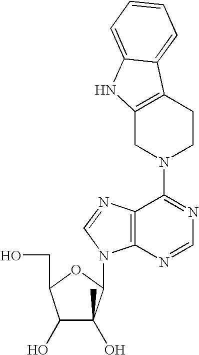 | 9-(2′-C-methyl-β-D-ribofuranosyl)- 6-(1,3,4,9-tetrahydro-beta-carbolin- 2-yl)purine |
| 34 | 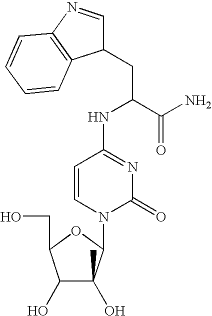 | 1-(2′-C-methyl-β-D-ribofuranosyl)- N⁴-[1-aminocarbonyl-2-(3H-indol- 3-yl)-ethyl]cytosine |
| 35 | 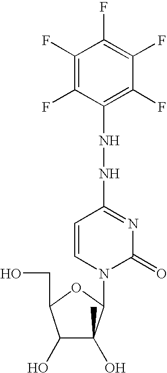 | 1-(2′-C-methyl-β-D-ribofuranosyl)- 4-(pentafluorophenyl-hydrazino)- pyrimidin-2-one |
| 37 | 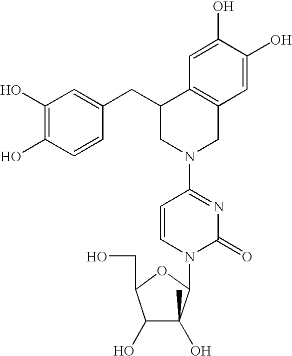 | 1-(2′-C-methyl-β-D-ribofuranosyl)- 4-[4-(3,4-dixydroxy-benzyl)-6,7- dihydroxy-3,4-dihydro-1H- isoquinolin-2-yl]-pyrimidin-2-one |
| 38 | 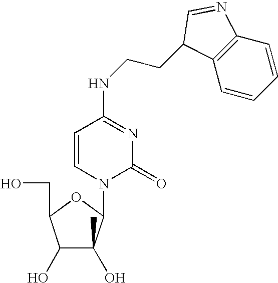 | 1-(2′-C-methyl-β-D-ribofuranosyl)- N⁴-[2-(3H-indol-3-yl)- ethyl]cytosine |
| 39 | 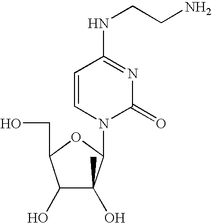 | 1-(2′-C-methyl-β-D-ribofuranosyl)- N⁴-(2-aminooethyl)cytosine |
| 40 | 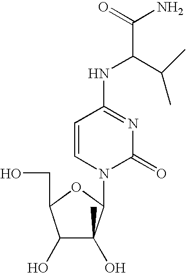 | 1-(2′-C-methyl-β-D-ribofuranosyl)- N⁴-(aminocarbonyl-isopropyl- methyl)cytidine |
| 53 | 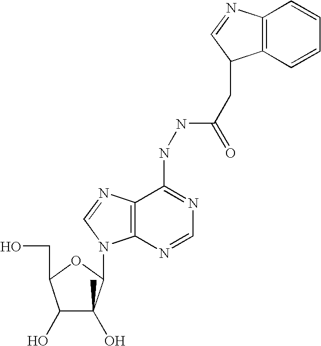 | 9-(2′-C-methyl-β-D-ribofuranosyl)- N⁶-{[(3H-indol-3-yl)-acetic acid]- hydrazide}adenine |
| 54 | 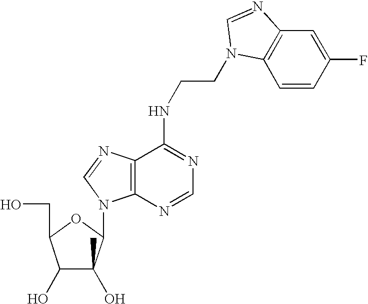 | 9-(2′-C-methyl-β-D-ribofuranosyl)- N⁶-[2-(5-fluoro-benzimidazol-1- yl)-ethyl]adenine |
| 55 | 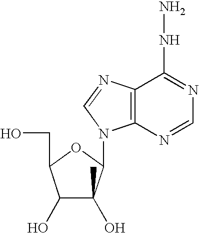 | 9-(2′-C-methyl-β-D-ribofuranosyl)- 6-hydrazino-purine |
| 56 | 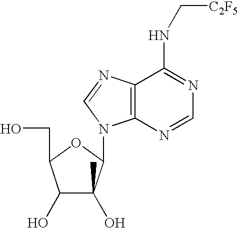 | 9-(2′-C-methyl-β-D-ribofuranosyl)- N⁶-(2,2,3,3,3,- pentafluoropropyl)adenine |
| 57 | 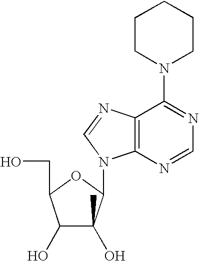 | 9-(2′-C-methyl-β-D-ribofuranosyl)- 6-(piperidin-1-yl)purine |
| 60 | 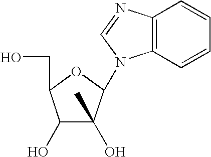 | 9-(2′-C-methyl-β-D-ribofuranosyl)- 1H-benzimidazol |
| 61 | 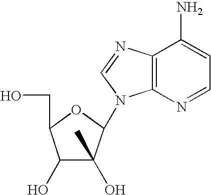 | 9-(2′-C-methyl-β-D-ribofuranosyl)- 3H-imidazo[4,5-b]pyridin-7-ylamine |
| 62 | 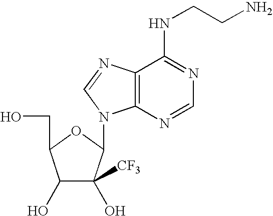 | 9-(2′-C-trifluoromethyl-β-D- ribofuranosyl)-N⁶-(2- aminoethyl)adenine |
| 63 | 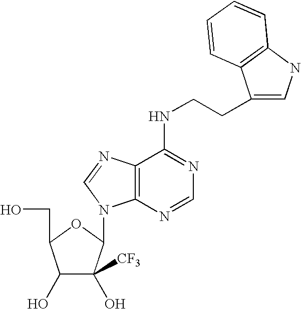 | 9-(2′-C-trifluoromethyl-β-D- ribofuranosyl)-N⁶-[2-(3H-indol-3- yl)-ethyl]adenine |
| 64 | 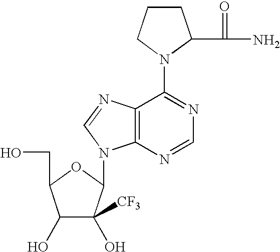 | 9-(2′-C-trifluoromethyl-β-D- ribofuranosyl)-6-[2-aminocarbonyl- (pyrrolidine-1-yl)]-purine |
| 66 | 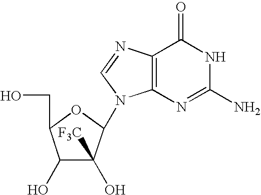 | 9-(2′-C-trifluoromethyl-β-D- ribofuranosyl)guanine |
| 67 | 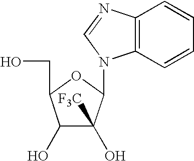 | 9-(2′-C-trifluoromethyl-β-D- ribofuranosyl)-1H-benzimidazole |
| 68 | 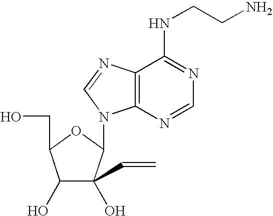 | 9-(2′-C-ethenyl-β-D-ribofuranosyl)- N⁶-(2-aminoethyl)adenine |
| 69 | 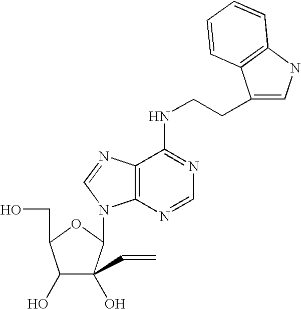 | 9-(2′-C-ethenyl-β-D-ribofuranosyl)- N⁶-[2-(3H-indol-3-yl)-ethyl]adenine |
| 70 | 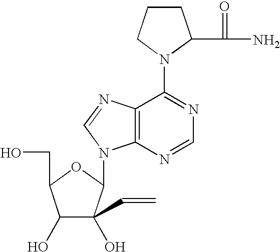 | 9-(2′-C-ethenyl-β-D-ribofuranosyl)- 6-[2-aminocarbonyl-(pyrrolidine-1- yl)]-purine |
| 73 | 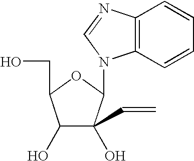 | 1-(2′-C-ethenyl-β-D-ribofuranosyl)- 1H-benzimidazole |
| 74 | 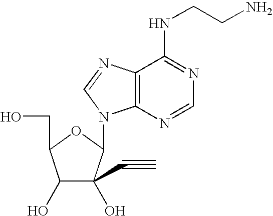 | 9-(2′-C-ethenyl-β-D-ribofuranosyl)- N⁶-(2-aminoethyl)adenine |
| 75 | 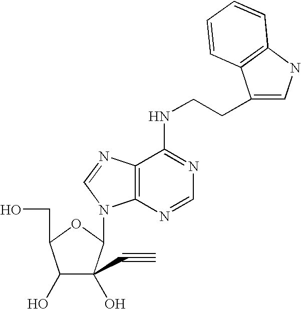 | 9-(2′-C-ethenyl-β-D-ribofuranosyl)- N⁶-[2-(3H-indol-3-yl)-ethyl]adenine |
| 76 | 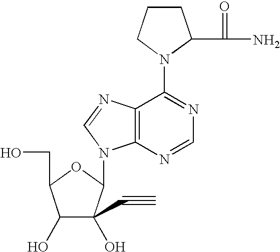 | 9-(2′-C-ethenyl-β-D-ribofuranosyl)- 6-[2-aminocarbonyl-(pyrrolidine-1- yl)]purine |
| 79 | 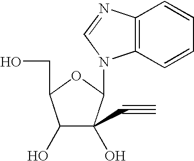 | 1-(2′-C-ethenyl-β-D-ribofuranosyl)- 1H-benzimidazole |
| 80 | 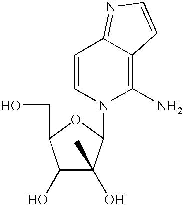 | 5-(2′-C-ethenyl-β-D-ribofuranosyl)- 5H-pyrrolo[3,2-c]pyridin-4-ylamine |
| 81 | 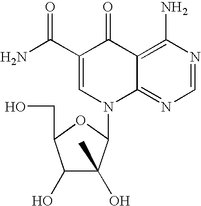 | 4-Amino-8-(2′-C-methyl-β-D- ribofuranosyl)-5-oxo-5,8-dihydro- pyrido[2,3-d]pyrimidine-6- carboxylic acid amide |
| 82 | 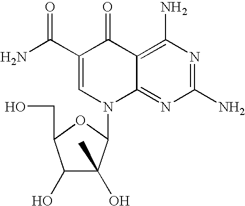 | 2,4-Diamino-8-(2′-C-methyl-β-D- ribofuranosyl)-5-oxo-5,8-dihydro- pyrido[2,3-e]pyrimidine-6- carboxylic acid amide |
| 83 | 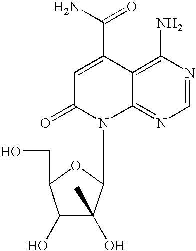 | 4-Amino-8-(2′-C-methyl-β-D- ribofuranosyl)-7-oxo-7,8-dihydro- pyrido[2,3-d]pyrimidine-5- carboxylic acid amide |
| 84 | 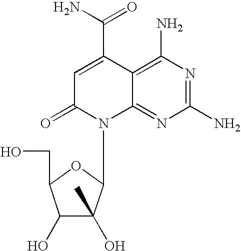 | 2,4-Diamino-8-(2′-C-methyl-β-D- ribofuranosyl)-7-oxo-7-8-dihydro- pyrido[2,3-d]pyrimidine-5- carboxylic acid amide |
| 85 | 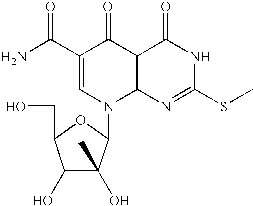 | 8-(2′-C-methyl-β-D-ribofuranosyl)- 2-methyolsulfanyl-4,5-dioxo-3,4,5,8-nl tetrahydro-pyrido[2,3-d]pyrimidine- 6-carboxylic acid amide |
| 86 | 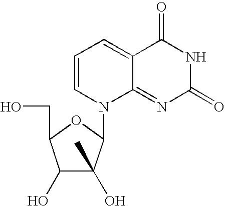 | 8-(2′-C-methyl-β-D-ribofuranosyl)- 8H-pyrido[2,3-d]pyrimidine-2,4- dione |
| 87 | 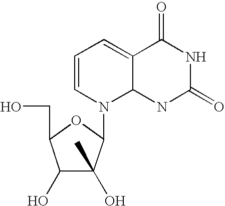 | 1-(2′-C-methyl-β-D-ribofuranosyl)- 1H-pyrido[2,3-d]pyrimidine-2,4- dione |
| 88 | 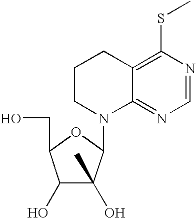 | 8-(2′-C-methyl-β-D-ribofuranosyl)- 4-methylsulfanyl-5,6.7,8-tetrahydro- pyrido[2,3-d]pyrimidine |
| 89 | 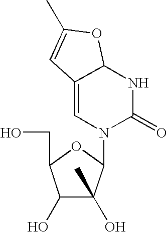 | 3-(2′-C-methyl-β-D-ribofuranosyl)- 6-methyl-3,7a-dihydro-1H-furo[2,3- d]pyrimidin-2-one |
| 90 | 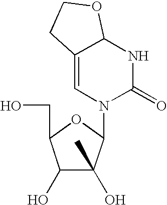 | 3-(2′-C-methyl-β-D-ribofuranosyl)- 3,5,6,7a-tetrahydro-1H-furo[2,3- d]pyrimidin-2-one |
| 92 | 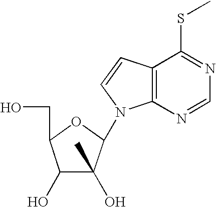 | 7-(2′-C-methyl-β-D-ribofuranosyl)- 4-methylsulfanyl-7H-pyrrolo[2,3- d]pyrimidine |
| 93 | 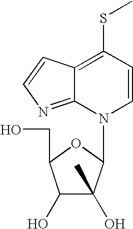 | 1-(2′-C-methyl-β-D-ribofuranosyl)- 4-methylsulfanyl-1H-pyrrolo[2,3- d]pyrimidine |
| 94 | 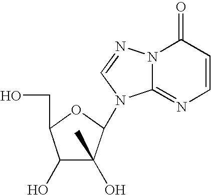 | 3-(2′-C-methyl-β-D-ribofuranosyl)- 3H-[1,2,4]triazolo[1,5-a]pyrimidin- 7-one |
| 95 | 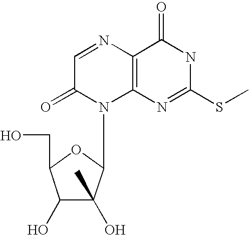 | 3-methyl-8-(2′-C-methyl-β-D- ribofuranosyl)-2-methylsulfanyl- 3H,8H-pteridine-4,7-dione |
| 96 | 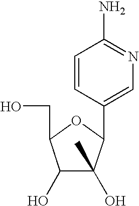 | 5-(2′-C-methyl-β-D-ribofuranosyl)- pyridin-2-ylamine |
| 97 | 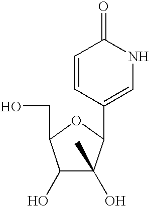 | 5-(2′-C-methyl-β-D-ribofuranosyl)- 1H-pyridin-2-one |
| 98 | 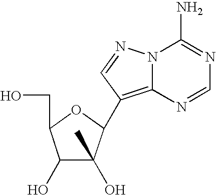 | 8-(2′-C-methyl-β-D-ribofuranosyl)- pyrazolo[1,5-a][1,3,5]triazin-4- ylamine |
| 99 | 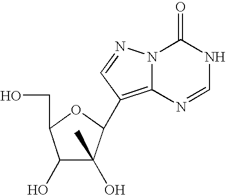 | 8-(2′-C-methyl-β-D-ribofuranosyl)- 3H-pyrazolo[1,5-a][1,3,5]triazin-4 one |
| 100 | 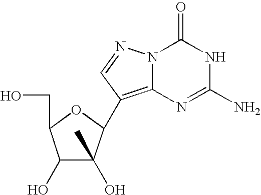 | 2-Amino-8-(2′-C-methyl-β-D- ribofuranosyl)-3H-pyrazolo[1,5- a][1,3,5]triazin-4-one |
| 104 | 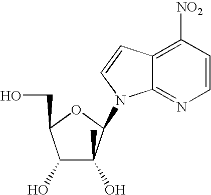 | 1-(2′-C-methyl-β-D-ribofuranosyl)- 4-nitroindole |
| 105 | 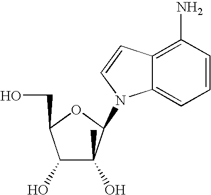 | 9-(2′-C-methyl-β-D-ribofuranosyl)- 4-aminoindole |
| 106 | 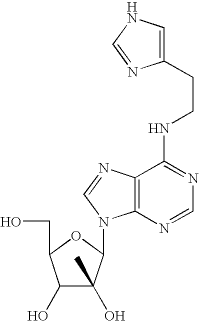 | 9-(2′-C-methyl-β-D-ribofuranosyl)- 6-[2-(1H-imidazol-4-yl)- ethyl]purine |
| 107 | 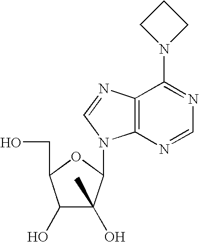 | 9-(2′-C-methyl-β-D-ribofuranosyl)- 6-(azetidin-1-yl)purine |
| 108 | 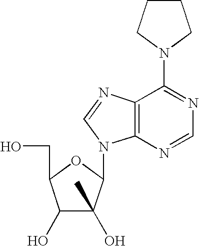 | 9-(2′-C-methyl-β-D-ribofuranosyl)- 6-(pyrrolidin-1-yl)purine |
| 110 | 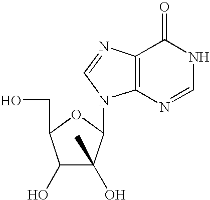 | (2′-C-methyl-β-D-ribofuranosyl)- hypoxanthine |
| 112 | 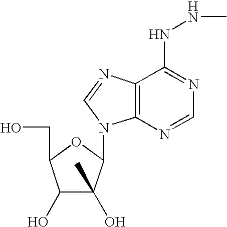 | 9-(2′-C-methyl-β-D-ribofuranosyl)- 6-methylhydrazinopurine |
| 113 | 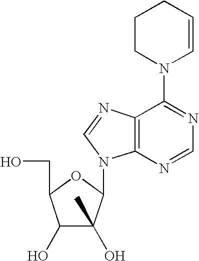 | 9-(2′-C-methyl-β-D-ribofuranosyl)- 6-(3,6-dihydro-2H-pyridin-1- yl(purine |
| 114 | 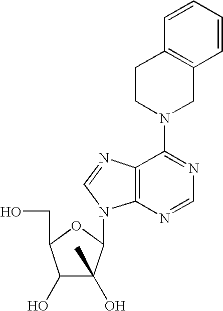 | 9-(2′-C-methyl-β-D-ribofuranosyl)- 6-(3,4-dihydro-1H-isoquinolin-2- yl)purine |
| 150 | 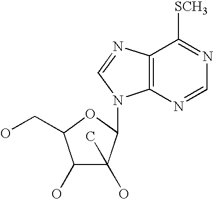 | 2′-C-methyl-β-D-ribofuranosyl-6- methythio-purine |
| 151 | 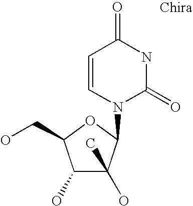 | 2′-C-methyl-β-D-ribofuranosyl- uracil |
| 152 | 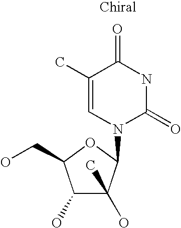 | 2′-C-methyl-β-D-ribofuranosyl- thymine |
| 155 | 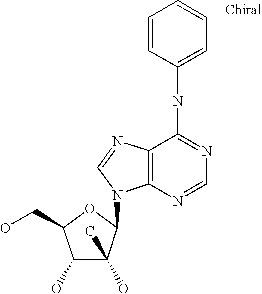 | 2′-C-methyl-β-D-ribofuranosyl-6- phenyladenin |
| 156 | 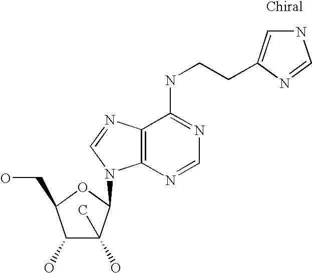 | 9-(2′-C-methyl-β-D-ribofuranosyl)- 6-(2-(1H-imidazo-1-4-yl)- ethylamino)purine |
| 157 | 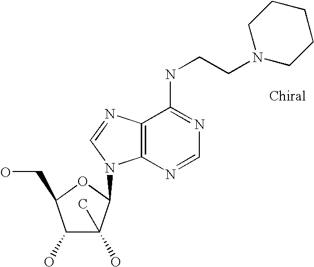 | 9-(2′-C-methyl-β-D-ribofuranosyl)- 6-(2-piperidi8n-1-yl- ethylamino)purine |
| 158 | 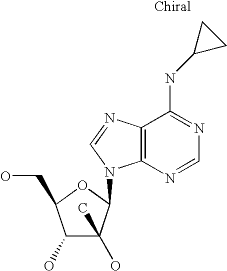 | 9-(2′-C-methyl-β-D-ribofuranosyl)- 6-(cyclopropylamino)purine |
| 159 | 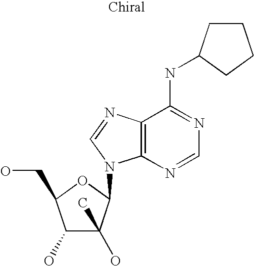 | 9-(2′-C-methyl-β-D-ribofuranosyl)- 6-(cyclopentylamino)purine |
| 160 | 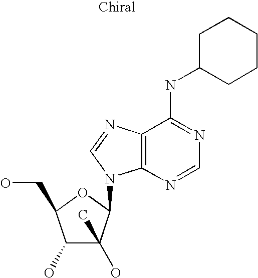 | 9-(2′-C-methyl-β-D-ribofuranosyl)- 6-(cyclohexylamino)purine |
| 161 | 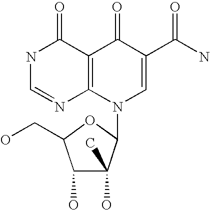 | 8-(3,4-dihydroxy-5-hydroxymethyl- 3-methyl-tetrahydro-furan-2-yl)-4,5- dioxo-3,4,5,8-tetrahydro-ppyrido[2,3-]d]pyrimidin-7-yl)-5-hydroxymethyl- 3-methyl-tetrahydro-furan-3,4-diol |
| 162 | 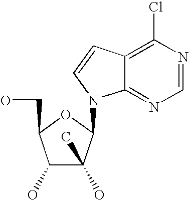 | 2-(4-Chloro-pyrrolo[2,3- d]pyrimidin-7-yl-5-hydroxymethyl- 3-methyl-tetrahydro-furan-3,4-diol |
| 163 | 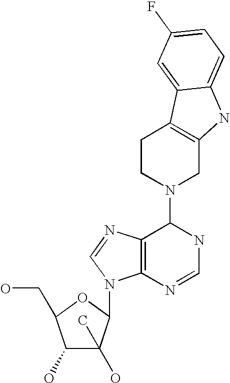 | 9-(2′-C-methyl-β-D-ribofuranosyl)- 6-(6-Fluoro-1,3,4,9-tetrahydro-β- carbolin-2-yl)purine |
| 164 | 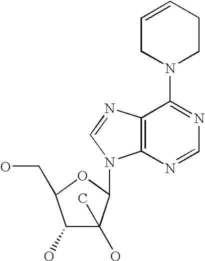 | 9-(2′-C-methyl-β-D-ribofuranosyl)- 6-(3,6-Dihydro-2H-pyridin-1- yl)purine |
| 165 | 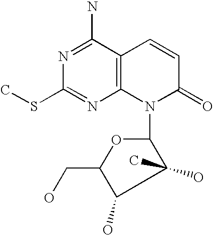 | 4-Amino-8-(3,4-dihydroxy-5- hydroxymethyl-3-methyl-tetrahydro- furan-2-yl)-2-methylsulfanyl-8H- pyrido[2,3-d]pyrimidin-7-one |
| 166 | 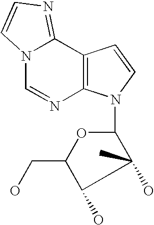 | 5-Hydroxymethyl-3-methyl-2- (1,3a,65,6-tetraaza-as-indacen-6-yl)- tetrahydro-furan-3,4-diol |
| 168 | 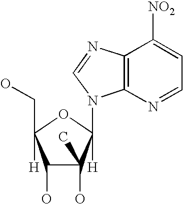 | 5-Hydroxymethyl-3-methyl-2-(7- nitro-imidaxo[4,5-b]-pyridin-3-yl)- tetrahydro-furan-3,4-diol |
| 169 | 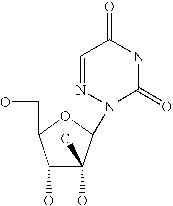 | 2-(3,4-Dihydroxy-5-hydroxymethyl- 3-methyl-tetrahydro-furan-2-yl)- 2H-[1,2,4]triazine-3,5-dione |
| 170 | 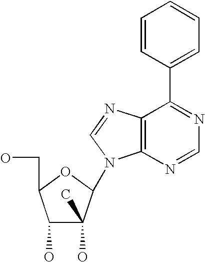 | 5-Hydroxymethyl-3-methyl-2-(6- phenyl-purin-9-yl)-tetrahydro-furan 3,4-diol |
| 171 | 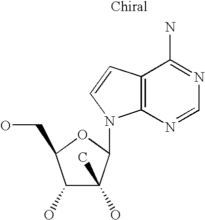 | 2-(4-Amino-pyrrolo[2,3- d]pyrimidin-7-yl)-5-hydroxymethyl- 3-methyl-tetrahydro-furan-3,4-diol |
| 172 | 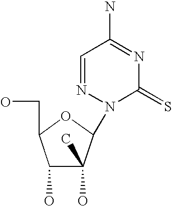 | 5-Amino-2-(3,4-dihydroxy-5- hydroxymethyl-3-methyl-tetrahydro- furan-2-yl)-4,5-dihydro-2H- [1,2,4]triaxine-3-thione |
| 173 | 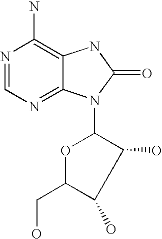 | 5-Amino-9-(3,4-dihydroxy-5- hydroxymethyl-3-methyl-tetrahydro- furan-2-yl)-7,9-dihydro-purin-8-one |
| 174 | 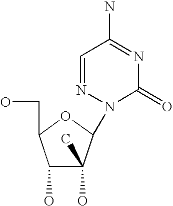 | 5-Amino-2-(3,4-dihydroxy-5- hydroxymethyl-3-methyl-tetrahydro- furan-2-yl)-2H-[1,2,4]triazin-3-one |
| 175 | 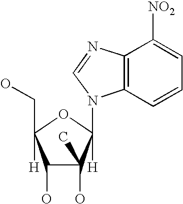 | 5-Hydroxymethy-3-methyl-2-(4- nitro-benzoimidazol-1-yl- tetrahydro-furan-3,4-diol |
| 176 | 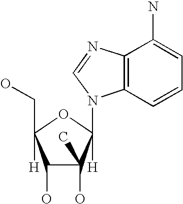 | 2-(4-AMino-benzoimidazol-1-yl)-5- hydroxymethyl-3-methyl-tetrahydro- furan-3,4-diol |
| 177 | 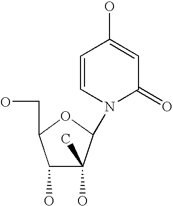 | 1-(3,4-Dihydroxy-5-hydroxymethyl- 3-methyl-tetrehydro-furan-2-yl)- 4-hydroxy-1H-puridin-2-one |
| 178 | 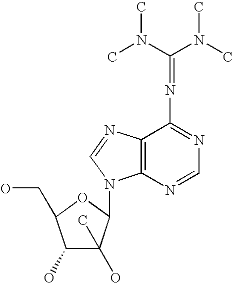 | 9-(2′C-methy-β-D-ribofuranosyl)- 6-(tetramethylguanidino)purine |
| 179 | 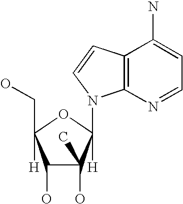 | 2-(4-Amino-pyrrolo[2,3-b]pyridin- 1-yl)-5-hydroxymethyl-3-methyl- tetrahydro-furan-3,4-diol |
| 182 | 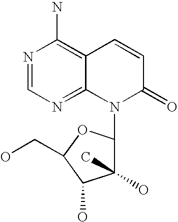 | 4-Amino-8-(3,4-dihydroxy-5- hydroxymethyl-3-methyl-tetrahydro- furan-2-yl)-8H-pyrido[2,3- d]pyrimidin-7-one |
| 183 | 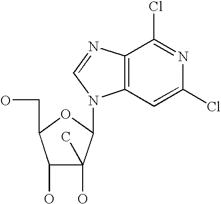 | 2-(2,4-Dichloro-5H-pyrrolo[3,2- d]pyrimidin-7-yl)-5-hydroxymethyl- 3-methyl-tetrahydro-furan-3,4-diol |
| 184a | 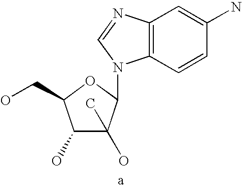 | 1-(2′-C-methy-β-D-ribofuranosyl)- 5-aminobenzimidazole and |
| 184b | 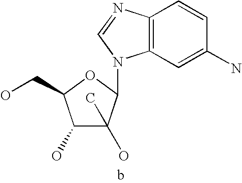 | 1-(2′-C-methy-β-D-ribofuranosyl)- 6-aminobenzimidazole |
| 185 | 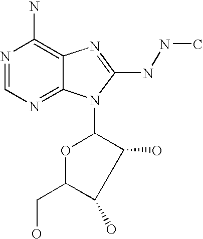 | 2-[6-Amino-8-(N′-methyl hydraxino-purin-9-yl]-5- hydroxymethyl-tetrahydro-furan 3,4-diol |
| 186 | 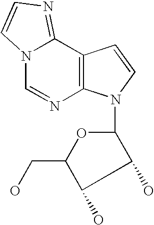 | 2-Hydroxymethyl-5-(1,3a,5,6- tetraaza-as-indacen-6-yl)-tetrahydro- furan-3,4-diol |
| 188 | 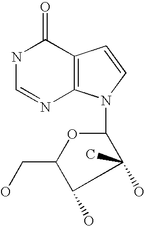 | 7-(3,4-Dihydroxy-5-hydroxymethyl- 3-methy-tetrahydeo-furan-2-yl)-3,7- dihydro-pyrrolo[2,3-d]pyrimidin-4- one |
| 189 | 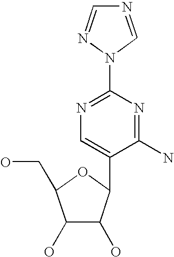 | 2-(4-Amino-2-[1,2,4]triazol-1-yl- pyrimidin-5-yl)-5-hydroxymethyl- tetrahydro-furan-3,4-diol |
| 190 | 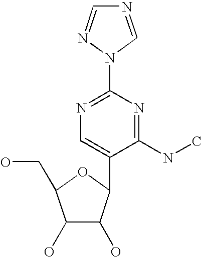 | 2-Hydroxymethy-5-(4- methylanimo-2-[1,2,4]triazol-1-yl- pyrimidin-5-yl)-tetrahydro-furan 3,4-diol |
| 200 | 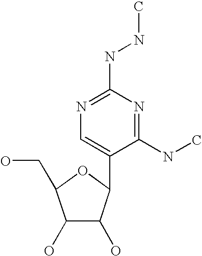 | 2-Hydroxymethyl-5-[4- methylamino-202(N′-methyl- hydrazino)-pyrimidin-5-yl]- tetrahydro-furan-3,4-diol |
| 201 | 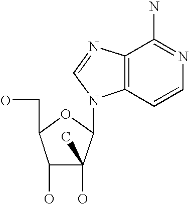 | 2-(4-Amino-5H-pyrrolo[3,2- d]pyrimidin-7-yl)-5-hydroxymethyl- 3-methyl-tetrahydeo-furan-3,4-diol |
| 203 | 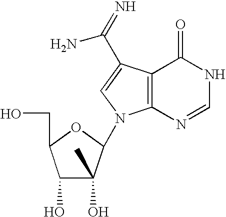 | 7-(3,4-Dihydroxy-5-hydroxymethyl- 3-methyl-tetrahydro-furan-2-yl)- 4-oxo-4,7-dihydro-3H-pyrrolo[2,3- d]pyrimidine-5-carboxamidine |
| 204 | 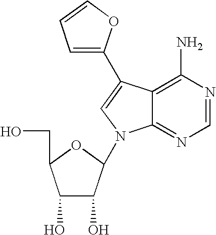 | 2-(4-Amino-5-furan-2-yl- pyrrolo[2,3-d]pyrimidin-7-yl)- 5-hydroxymethyl-tetrahydro-furan- 3,4-diol |
| 205 | 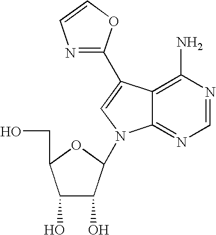 | 2-(4-Amino-5-oxazol-2-yl- pyrrolo[2,3-d]pyrimidin-7-yl)- 5-hydroxymethyl-tetrahydro-furan- 3,4-diol |
| 206 | 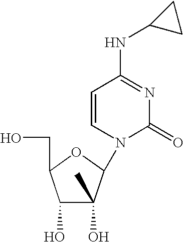 | 4-Cyclopropylamino-1-(3,4- dihydroxy-5-hydroxymethyl-3- methyl-tetrahydro-furan-2-yl)-1H- pyrimidin-2-one |
| 207 | 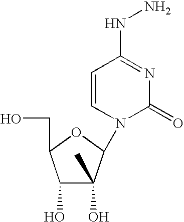 | 1-(3,4-Dihydroxy-5-hydroxymethyl- 3-methyl-tetrahydro-furan-2-yl)- 4-hydrazino-3,4-dihydro-1H- pyrimidin-2-one |
| 208 | 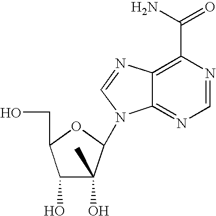 | 2′-C-methyl-β-D-ribofuranosyl- purine-6-carboxamide |
| 209 | 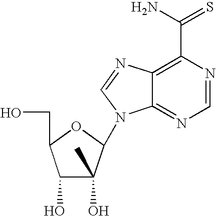 | 9-(3,4-Dihydroxy-5-hydroxymethyl- 3-methyl-tetrahydeo-furan-2-yl)-9H- purine-6-carbothioic acid amide |
| 210 | 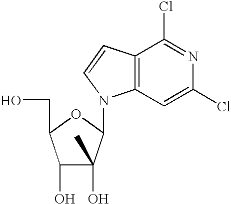 | 2-(4,6-Dichloro-pyrrolo[3,2- c]pyridin-1-yl)-5-hydroxymethyl-3- methyl-tetrahydro-furan-3,4-diol |
| 211 | 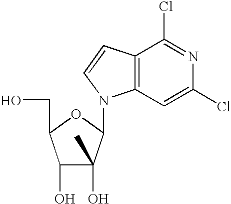 | 2-(4-Amino-6-chloro-pyrrolo[3,2- c]pyridin-1-yl)-5-hydroxymethyl-3- methyl-tetrahydro-furan-3,4-diol |
| 212 | 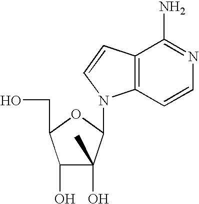 | 2-(4-Amino-pyrrolo[3,2- c]pyridin-1-yl)-5-hydroxymethyl-3- methyl-tetrahydro-furan-3,4-diol |
| 213 | 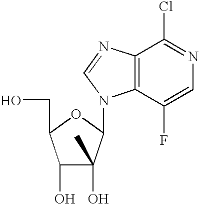 | 4-Chloro-7-fluoro-1-(2′-C-methyl-β- D-ribofuranosyl)imidazo[4,5- c]pyridine |
| 214 | 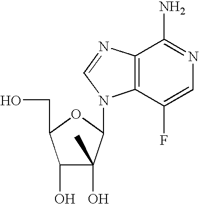 | 4-Amino-7-fluoro-1-(2′-C-methyl-β- D-ribofuranosyl)imidazo [4,5-c]pyridine |
| 215 | 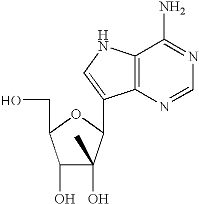 | 2-(4-Amino-5H-pyrrolo[3,2- d]pyrimidin-7-yl)-5hydroxymethyl- 3-methyl-tetrahydro-furan-3,4-diol |
| 216 | 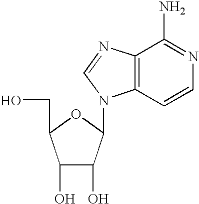 | 4-Amino-1-(β-D- ribofuranosyl)imidazo[4,5- c]pyridine |
| 217 | 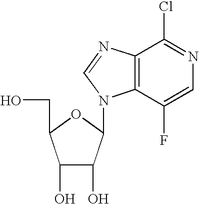 | 4-Chloro-7-fluoro-1-(β-D- ribofuranosyl)imidazo[4,5- c]pyridine |
| 218 | 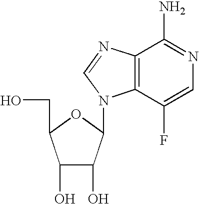 | 4-Amino-7-fluoro-1-(β-D- ribofuranosyl)imidazo[4,5- c]pyridine |
| 219 | 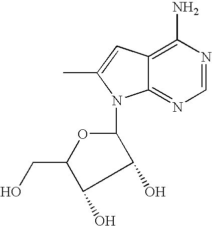 | 2-(4-Amino-6-methyl-pyrrolo[2,3-d]pyrimidin-7-yl)-5-hydroxymethyol-tetrahydro-furan-3,4-diol |
| 220 | 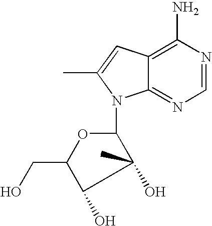 | 2-(4-Amino-6-methyl-pyrrolo[2,3-d]pyrimidin-7-yl)-5-hydroxymethyol-tetrahydro-furan-3,4-diol |
| 221 | 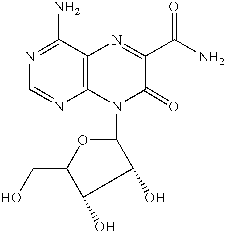 | 4-Amino-8-(3,4-dihydroxy-5- hydroxymethyl-tetrahydro-furan-2- yl)-7-oxo-7,8-dihydro-pteridin-6- carboxylic acide amide |
| 222 | 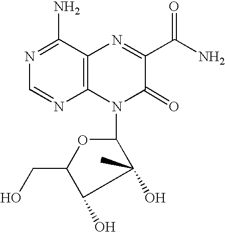 | 4-Amino-8-(3,4-dihydroxy-5-hydroxymethyl-3-methyl-tetrahydro-furan-2-yl)-7-oxo-7,8-dihydro-pteridine-6-carboxylic acid amide |
| 223 | 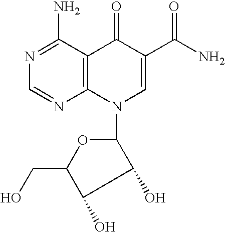 | 4-Amino-8-(3,4-dihydroxy-5- hydroxymethyl-3-methyl-tetrahydro- furan-2-yl)-5-oxo-5,8-dihydro-pyrido[2,3-d]-pyrimidine-6- carboxylic acid amide |
| 224 | 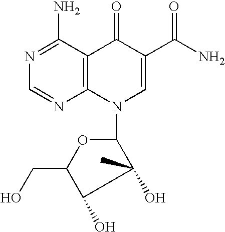 | 4-Amino-8-(3,4-dihydroxy-5- hydroxymethyl-3-methyl-tetrahydro- furan-2-yl)-5-oxo-5,8-dihydro-pyrido[2,3-d]-pyrimidine-6- carboxylic acid amide |
| 225 | 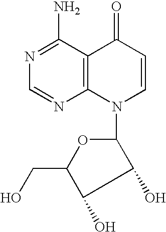 | 4-Amino-8-(3,4-dihydroxy-5-hydroxymethyl-tetrahydro-furan-2-yl)-5-oxo-5,8-dihydro-pyrido[2,3-d]-pyrimidine-6-carboxylic acid amide |
| 226 | 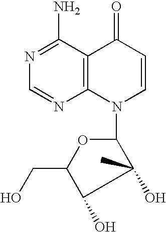 | 4-Amino-8-(3,4-dihydroxy-5-hydroxymethyl-3-methyl-tetrahydro-furan-2-yl)-8H-pyrido-[2,3-d]-pyrimidine-5-onecarboxylic acid amide |
| 227 | 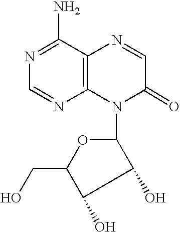 | 4-Amino-8-(3,4-dihydroxy-5- hydroxymethyl-tetrahydro-furan-2- yl)-8H-pteridin-7-one |
| 228 | 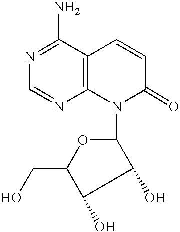 | 4-Amino-8-(3,4-dihydroxy-5- hydroxymethyl-tetrahydro-furan-2- yl)-8H-pyrido[2,3-d]pyrimidin-7- one |
| 229 | 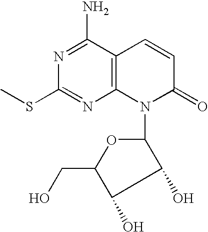 | 4-Amino-8-(3,4-dihydroxy-5- hydroxymethyl-tetrahydro-furan-2- yl)-methylsulfanyl-8H-pyrido[2,3- d]pyrimidin-7-one |
| 230 | 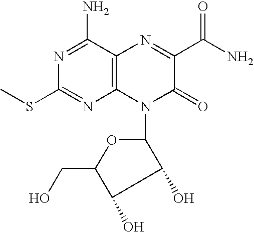 | of 4-Amino-8-(3,4-dihydroxy-5-hydroxymethyl-3-methyl-tetrahydro-furan-2-yl)-methylsulfanyl-7-oxo7,8-dihydro-pteridine-6-carboxylicacid amide |

This invention is also directed to pharmaceutical compositions comprising a pharmaceutically acceptable diluent and a therapeutically effective amount of a compound of Formula Ia, Ib, Ic, II, IIA, III, or IV or mixtures of one or more of such compounds.

This invention is still further directed to methods for treating HCV in mammals which methods comprise administering to a mammal diagnosed with HCV or at risk of developing HCV a pharmaceutical composition comprising a pharmaceutically acceptable diluent and a therapeutically effective amount of a compound of Formula Ia, Ib, Ic, II, IIA, III, or IV or mixtures of one or more of such compounds.

In still another of its method aspects, this invention is directed to a method for preparing the compounds of formula III:

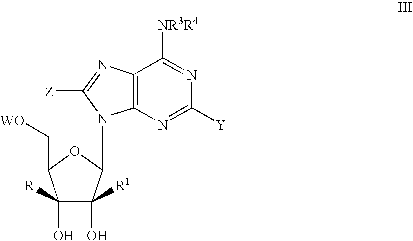

where R, R¹, R³, R⁴, W, X, Y and Z are as defined above which method comprises:

(a) oxidizing a compound of formula IV

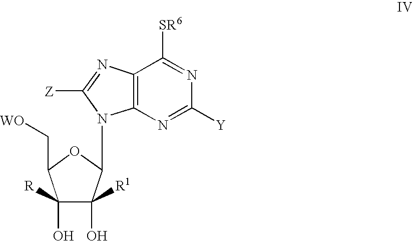

where R⁶ is selected from the group consisting of alkyl and aryl;

(b) oxidizing the thio group to a sulfoxide or sulfone; and

(c) contacting the oxidized compound prepared in (b) above with at least a stoichiometric equivalent of HNR³R⁴ under conditions which result in formation of a compound of formula II

wherein R³ and R⁴ are independently selected from the group consisting of hydrogen, alkyl, substituted alkyl, alkenyl, substituted alkenyl, alkynyl and substituted alkynyl, aryl, substituted aryl, heteroaryl, substituted heteroaryl, heterocyclic, substituted heterocyclic and where R³ and R⁴ are joined to form, together with the nitrogen atom bond thereto, a heterocyclic group.

### DETAILED DESCRIPTION OF THE INVENTION

The invention is directed to compounds, compositions and methods for treating hepatitis C virus infections. However, prior to describing this invention in detail, the following terms will first be defined:

Definitions

As used herein, “alkyl” refers to alkyl groups having from 1 to 10 carbon atoms, preferably from 1 to 5 carbon atoms and more preferably I to 3 carbon atoms. This term is exemplified by groups such as methyl, ethyl, n-propyl, iso-propyl, n-butyl, t-butyl, n-pentyl and the like.

“Substituted alkyl” refers to an alkyl group having from 1 to 3, and preferably 1 to 2, substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Alkoxy” refers to the group “alkyl-O—” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)— cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acylamino” refers to the group —C(O)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” refers to alkenyl group preferably having from 2 to 6 carbon atoms and more preferably 2 to 4 carbon atoms and having at least 1 and preferably from 1-2 sites of alkenyl unsaturation.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Alkynyl” refers to alkynyl group preferably having from 2 to 6 carbon atoms and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Amino” refers to the group —NH₂.

“Substituted amino” refers to the group —NR R where R and R are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R and R are joined, together with the nitrogen bound thereto to form a heterocyclic or substituted heterocylic group provided that R and R are both not hydrogen. When R is hydrogen and R is alkyl, the substituted amino group is sometimes referred to herein as alkylamino. When R and R are alkyl, the substituted amino group is sometimes referred to herein as dialkylamino.

“Amidino” refers to groups with the formula —C(═NR′″)NR′R″ where R′, R″ and R′″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R′ and R″ are joined, together with the nitrogen bound thereto to form a heterocyclic, substituted heterocyclic, heteroaryl or substituted heteroaryl group. The term amidino also refers to reverse amidino structures of the formula:

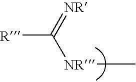

where R″″ is an alkyl or substituted alkyl group as defined above and R′″ and R′ are as defined above.

“Guanidino” refers to groups with the formula —NHC(═NR′″)NR′R″ where R′, R″ and R′″ are as defined above for amidino.

“Aminoacyl” refers to the groups —NRC(O)alkyl, —NRC(O)substituted alkyl, —NRC(O)cycloalkyl, —NRC(O)substituted cycloalkyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like). Preferred aryls include phenyl and naphthyl.

“Substituted aryl” refers to aryl groups which are substituted with from 1 to 3 substituents, and preferably 1 to 2 substituents, selected from the group consisting of hydroxy, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxyl, carboxyl esters, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Aryloxy” refers to the group aryl-O— that includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 3 substituents on either or both aryl rings as defined above for substituted aryl.

“Carboxyl” refers to —COOH or salts therof.

“Carboxyl esters” refers to the groups —C(O)O-alkyl, —C(O)O-substituted alkyl, —C(O)Oaryl, and —C(O)O-substituted aryl wherein alkyl, substituted alkyl, aryl and substituted aryl are as defined herein.

“Cycloalkyl” refers to cyclic alkyl groups of from 3 to 10 carbon atoms having single or multiple cyclic rings including, by way of example, adamantyl, cyclopropyl, cyclobutyl, cyclopentyl, cyclooctyl and the like.

“Cycloalkenyl” refers to cyclic alkenyl groups of from 4 to 10 carbon atoms having single or multiple cyclic rings and further having at least 1 and preferably from 1 to 2 internal sites of ethylenic (C═C) unsaturation.

“Substituted cycloalkyl” and “substituted cycloalkenyl” refers to an cycloalkyl or cycloalkenyl group, having from 1 to 5 substituents selected from the group consisting of oxo (═O), thioxo (═S), alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Cycloalkoxy” refers to —O-cycloalkyl groups.

“Substituted cycloalkoxy” refers to —O-substituted cycloalkyl groups.

“Halo” or “halogen” refers to fluoro, chloro, bromo and iodo and preferably is fluoro or chloro.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur within the ring. Such heteroaryl groups can have a single ring (e.g., pyridyl or furyl) or multiple condensed rings (e.g., indolizinyl or benzothienyl). Preferred heteroaryls include pyridyl, pyrrolyl, indolyl, thiophenyl, and furyl.

“Substituted heteroaryl” refers to heteroaryl groups that are substituted with from 1 to 3 substituents selected from the same group of substituents defined for substituted aryl.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl.

“Substituted heterocyclic” refers to heterocycle groups that are substituted with from 1 to 3 of the same substituents as defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“Phosphate” refers to the groups —OP(O)(OH)₂ (monophosphate), —OP(O)(OH)OP(O)(OH)₂ (diphosphate) and —OP(O)(OH)OP(O)(OH)OP(O)(OH)₂ (triphosphate) or salts thereof including partial salts thereof.

“Phosphonate” refers to the groups —OP(OR)(OH) or —OP(OR)(OR) or salts thereof including partial salts thereof.

“Thiol” refers to the group —SH.

“Thioalkyl” or “alkylthioether” or “thioalkoxy” refers to the group —S-alkyl.

“Substituted thioalkyl” or “substituted alkylthioether” or “substituted thioalkoxy” refers to the group —S-substituted alkyl.

“Thiocycloalkyl” refers to the groups —S-cycloalkyl and “substituted thiocycloalkyl” refers to the group —S-substituted cycloalkyl.

“Thioaryl” refers to the group —S-aryl and “substituted thioaryl” refers to the group —S-substituted aryl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

The term “amino acid” refers to a-amino acids of the formula H₂NCH(R⁷)COOH where R⁷ is alkyl, substituted alkyl or aryl. Preferably, the α-amino acid is one of the twenty naturally occurring L amino acids.

The term “carbohydrate” refers to oligosaccharides comprising from 2 to 20 saccharide units. The particular saccharide units employed are not critical and include, by way of example, all natural and synthetic derivatives of glucose, galactose, N-acetylglucosamine, N-acetylgalactosamine, fucose, sialic acid, and the like. In addition to being in their pyranose form, all saccharide units described herein are in their D form except for fucose which is in its L form.

The term “lipid” is an art recognized term defined, for example, by Lehninger, Biochemistry, 1970, at pages 189 et seq. which is incorporated herein by reference in its entirety.

The term “peptide” refers to polymers of a-amino acids comprising from about 2 to about 20 amino acid units, preferably from about 2 to about 10, more preferably from about 2 to about 5.

The term “stablilized phosphate prodrug” refers to mono-, di- and tri-phosphate groups having one or more of the hydroxyl groups pendent thereto converted to an alkoxy, a substituted alkoxy group, an aryloxy or a substituted aryloxy group.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

Similarly, it is understood that the above definitions are not intended to include impermissible substitution patterns (e.g., methyl substituted with fluoro groups or a hydroxyl group alpha to ethenylic or acetylenic unsaturation). Such impermissible substitution patterns are well known to the skilled artisan.

General Synthetic Methods

The compounds of this invention may be prepared by various methods known in the art of organic chemistry in general and nucleoside and nucleotide analogue synthesis in particular. The starting materials for the syntheses are either readily available from commercial sources or are known or may be prepared by techniques known in the art. General reviews of the preparation of nucleoside and nucleotide analogues are included in the following:

Michelson A. M. “The Chemistry of Nucleosides and Nucleotides,” Academic Press, New York, 1963.

Goodman L. “Basic Principles in Nucleic Acid Chemistry,” Academic Press, New York, 1974, vol. 1, Ch. 2.

“Synthetic Procedures in Nucleic Acid Chemistry,” Eds. Zorbach W. & Tipson R., Wiley, New York, 1973, vol. 1 & 2.

The synthesis of carbocyclic nucleosides has been reviewed by Agrofoglio et al. (Tetrahedron, 1994, 50, 10611).

The compounds of the present invention may be prepared using methods outlined in U.S. Provisional Application Serial No. 60/378,624, incorporated herein by referenence in its entirety.

The strategies available for synthesis of compounds of this invention include:

A. General Synthesis of 2′—C-Branched Nucleosides

2′—C-Branched ribonucleosides of the following structures:

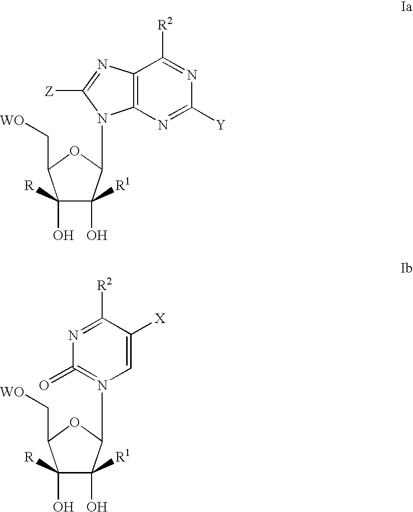

where R¹, R², W, X, Y and Z are as defined above, can be prepared by one of the following general methods.

1. Convergent Approach: Glycosylation of Nucleobase with Appropriately Modified Sugar

The key starting material of this process is an appropriately substituted sugar with 2′-OH and 2′-H with the appropriate leaving group, for example an acyl group or a chloro, bromo, fluoro or iodo. The sugar can be purchased or can be prepared by any known means including standard epimerization, substitution, oxidation and reduction techniques. For example, commercially available 1,3,5-tri-O-benzoyl-α-D-ribofuranose (Pfanstiel Laboratories, Inc.) can be used. The substituted sugar can then be oxidized with the appropriate oxidizing agent in a compatible solvent at a suitable temperature to yield the 2′-modified sugar. Possible oxidizing agents are, for example, Dess-Martin periodine reagent, Ac₂O+DCC in DMSO, Swern oxidation (DMSO, oxalyl chloride, triethylamine), Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Coupling of an organometallic carbon nucleophile, such as a Grignard reagent, an organolithium, lithium dialkylcopper or R¹—SiMe₃ in TBAF with the ketone with the appropriate non-protic solvent at a suitable temperature, yields the 2′-alkylated sugar. For example, R¹MgBr/TiCl₄ or R¹MgBr/CeCl₃ can be used as described in Wolfe et al. 1997. J. Org. Chem. 62: 1754-1759. The alkylated sugar can be optionally protected with a suitable protecting group, preferably with an acyl, substituted alkyl or silyl group, by methods well known to those skilled in the art, as taught by Greene et al. Protective Groups in Organic Synthesis, John Wiley and Sons, Second Edition, 1991.

The optionally protected sugar can then be coupled to the purine or pyrimidine base by methods well known to those skilled in the art, as taught by Townsend Chemistry of Nucleosides and Nucleotides, Plenum Press, 1994. For example, an acylated sugar can be coupled to a silylated base with a Lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

Scheme 1 below describes the alternative synthesis of a protected sugar that is useful for coupling to bases where the connection to the base is on a carbon atom instead of a nitrogen atom.

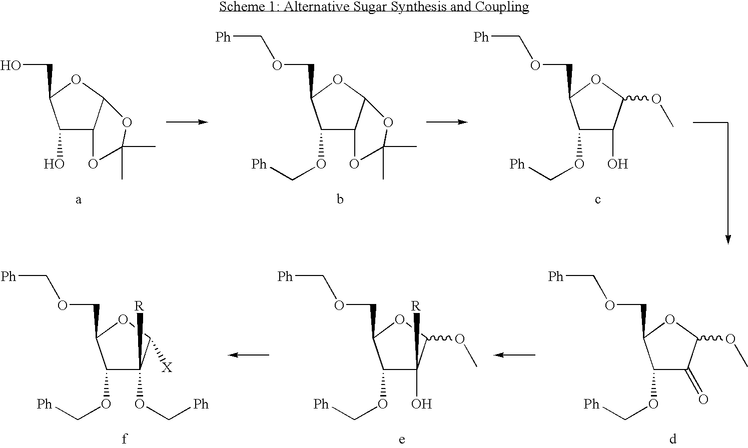

Formation of sugar a in Scheme 1, above, is accomplished as described by Mandal, S. B., et al., Synth. Commun., 1993, 9, page 1239, starting from commercial D-ribose. Protection of the hydroxyl groups to form sugar b is described in Witty, D. R., et al., Tet. Lett., 1990, 31, page 4787. Sugar c and d are prepared using the method of Ning, J. et al., Carbohydr. Res., 2001, 330, page 165, and methods described herein. R, in Sugar e can be hydrogen, alkyl, substituted alkyl, alkenyl, substituted alkenyl, alkynyl, and substituted alkynyl. Particularly preferred R groups are methyl, trifluoromethyl, alkenyl and alkynyl. Sugar e is prepared by using a modification of the Grignard reaction withn RMgBr or other appropriate organometallic as described herein (with no Titanium/cerium needed). Finally the halogenated sugar used in the subsequent coupling reaction is prepared using the same protection method as used in to make sugar b above. The halogenation is described in Seela.¹⁷

Subsequently, any of the described nucleosides can be deprotected by methods well known to those skilled in the art, as taught by Greene et al. Protective Groups in Organic Synthesis, Jon Wiley and Sons, Second Edition, 1991.

In a particular embodiment, the 2′-C-branched ribonucleoside is desired.

2. Linear Approach: Modification of a Pre-Formed Nucleoside

The key starting material for this process is an appropriately substituted nucleoside with a 2′-OH and 2′-H. The nucleoside can be purchased or can be prepared by any known means including standard coupling techniques. The nucleoside can be optionally protected with suitable protecting groups, preferably with acyl, substituted alkyl or silyl groups, by methods well known to those skilled in the art, as taught by Greene et al. Protective Groups in Organic Synthesis, John Wiley and Sons, Second Edition, 1991.

The appropriately protected nucleoside can then be oxidized with the appropriate oxidizing agent in a compatible solvent at a suitable temperature to yield the 2′-modified sugar. Possible oxidizing agents are, for example, Dess-Martin periodine reagent, Ac₂O+DCC in DMSO, Swern oxidation (DMSO, oxalyl chloride, triethylamine), Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂ ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide. Coupling of an organometallic carbon nucleophile, such as a Grignard reagent, an organolithium, lithium dialkylcopper or R′—SiMe₃ in TBAF with the ketone with the appropriate non-protic solvent at a suitable temperature, yields the appropriate substituted nucleoside.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 2′-C-branched ribonucleoside is desired. In another embodiment of the invention, the L-enantiomers are desired. Therefore, the L-enantiomers can be corresponding to the compounds of the invention can be prepared following the same foregoing general methods, beginning with the corresponding L-sugar or nucleoside L-enantiomer as starting material.

B. General Synthesis of 3′-C-Branched Nucleosides

3′-C-Branched ribonucleosides of the following structure:

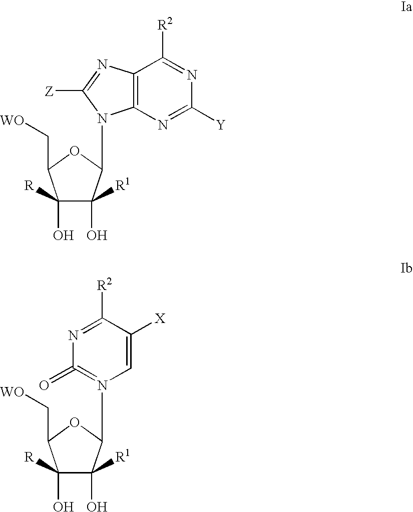

where R, R², W, X, Y and Z are as defined above, can be prepared by one of the following general methods.

1. Convergent Approach: Glycosylation of the nucleobase with an Appropriately Modified Sugar

The starting material for this process is an appropriately substituted sugar with a 3′-OH and 3′-H, with the appropriate leaving group, for example an acyl group, methoxy group or a chloro, bromo, fluoro, iodo. The sugar can be purchased or can be prepared by any known means including standard epimerization, substitution, oxidation and reduction techniques. The substituted sugar can then be purchased or can be prepared by any known means including standard epimerization, substitution, oxidation and reduction techniques. The substituted sugar can then be oxidized with the appropriate oxidizing agent in a compatible solvent at a suitable temperature to yield the 3′-modified sugar. Possible oxidizing agents are, for example, Dess-Martin periodine reagent, Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Then coupling of an organometallic carbon nucleophile, such as a Grignard reagent, an organolithium, lithium dialkylcopper or R-SiMe₃ in TBAF with the ketone with the appropriate non-protic solvent at a suitable temperature, yields the 3′-C-branched sugar. For example, RMgBr/TiCl₄ or RMgBr/CeCl₃ can be used as described in Wolfe et al. 1997. J. Org. Chem. 62: 1754-1759. The 3′-C-branched sugar can be optionally protected with a suitable protecting group, preferably with an acyl or silyl group, by methods well known to those skilled in the art, as taught by Greene et al. Protective Groups in Organic Synthesis, John Wiley and Sons, Second Edition, 1991.

The optionally protected sugar can then be coupled to the base by methods well known to those skilled in the art, as taught by Townsend Chemistry of Nucleosides and Nucleotides, Plenum Press, 1994. For example, an acylated sugar can be coupled to a silylated base with a Lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 3′-C-branched ribonucleoside is desired. Alternatively, deoxyribonucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

2. Linear Approach: Modification of a Pre-Formed Nucleoside

The key starting material for this process is an appropriately substituted nucleoside with a 3′-OH and 3′-H. The nucleoside can be purchased or can be prepared by any known means including standard coupling techniques. The nucleoside can be optionally protected with suitable protecting groups, preferably with acyl or silyl groups, by methods well known to those skilled in the art, as taught by Greene et al. Protective Groups in Organic Synthesis, John Wiley and Sons, Second Edition, 1991.

The appropriately protected nucleoside can then be oxidized with the appropriate oxidizing agent in a compatible solvent at a suitable temperature to yield the 3′-modified sugar. Possible oxidizing agents are, for example, Dess-Martin periodine reagent, Jones reagent (a mixture of chromic acid and sulfuric acid), Collins's reagent (dipyridine Cr(VI) oxide), Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 3′-C-branched ribonucleoside is desired. Alternatively, deoxyribonucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

In another embodiment of the invention, the L-enantiomers are desired. Therefore, the L-enantiomers can be corresponding to the compounds of the invention can be prepared following the same foregoing general methods, beginning with the corresponding L-sugar or nucleoside L-enantiomer as starting material.

C. General Synthesis of Purine Bases of Formula Ia and Pyrimidines Bases of Formula Ib

The purine bases of formula I-IVa and pyrimidines bases of formula I-IVb for above condensation reactions can be obtained commercially or can be prepared by procedures known to the art.

The preparation of purine bases of formula I-IVa is reviewed by G. Shaw in “Comprehensive Heterocyclic Chemistry,” Pergamon Press, Vol. 5, chapter 4.09, p. 449 and “Comprehensive Heterocyclic Chemistry II38 Pergamon Press, Vol. 7, chapter 7.11, p.397.

The preparation of pyrimidines bases of formula I-IVb is reviewed by Brown D. “The Chemistry of Heterocyclic Compounds—The Pyrimidines” 1962 and Supplement 1, 1970 John Wiley and Sons, New York, by Brown D. in “Comprehensive Heterocyclic Chemistry,” Pergamon Press Vol. 7, chapter 4.09, p. 499 and by K. Unheim and T. Benneche in “Comprehensive Heterocyclic Chemistry II” Pergamon Press Vol. 6 chapter 6.02, p. 93.

For example, the appropriate purine base of formula I-IVa may be prepared from the corresponding purine wherein the 2, 6 or 8 position of the purine base is substituted with a suitable leaving group such as halogen or sulphonate. Such purine precursors bearing leaving groups are available commercially, e.g. 6-chloropurine (Aldrich Chemical Company), 2,6-dichloropurine (Aldrich Chemical Company), 2-chloro-6-aminopurine (Aldrich Chemical Company), 8-bromoadenine (Sigma-Aldrich Company Limited) or obtained by procedures known in the art. For example 2- and 6-chloro substituted purines can be prepared by chlorination of the corresponding 2 and 6-hydroxypurines respectively by the use of chlorinating agents such as phosphorus oxychloride (Bakuni et al. Indian J. Chem., Sect B 1984, 23, 1286; LaMontagne et al. J. Heterocycl. Chem. 1983, 20, 295) while introduction of a bromine into the 8-position of purines can be accomplished by direct bromination using brominating agents such as, for example, bromine (Mano et al, Chem Pharm Bull 1983, 31, 3454) or N-bromosuccinimide (Kelley et al. Heterocycl. Chem. 1990, 27, 1505). The purines where the 6-substituent is alkoxy, aryloxy, SH, alkylthio, arylthio, alkylamino, cycloalkylamino, saturated cyclic amino, nitrogen linked heteroaromatic, hydroxylamino, alkoxylamino, hydrazine, alkylhydrazino may be prepared by treatment of the corresponding 6-halopurine with the appropriate alkoxides, thiols, amines, nitrogen containing heterocycles, hydroxylamines and hydrazines, (for example, Chae et al. J Med Chem, 1994, 37, 342; Niebch and Schneider, Z. Naturforsch. B.Anorg. Chem. Org. Chem. Biochem. Biophys. Biol. 1972, 27, 675; LaMontagne et al., Heterocycl Chem 1983, 20, 295; Estep et al J Med Chem 1995, 38, 2582). Similarly, 2-substituted purines can be prepared from the corresponding 2-halopurine, for example, purines where the 2-substituent is alkoxy, aryloxy, SH, alkythio, arylthio or NR³R⁴ can be prepared from the corresponding 2-halopurine by treatment with alkoxides, thiols or amines (e.g. Barlin and Fenn, Aust J Chem, 1983, 36, 633; Nugiel et al., J Org Chem, 1997, 62, 201). Similarly, 8-substitued purines can be prepared from the corresponding 8-halopurines. For example purines where the 8-substituent is alkoxy, aryloxy, SH, alkythio, arylthio or NR³R⁴ can be prepared by treatment of the corresponding 8-bromopurine with the appropriate alkoxides, thiols or amines (Xing et al, Tetrahedron Lett, 1990, 31, 5849; Mano et al, Chem Pharm Bull 1983, 31, 3454). Where the 2, 6 or 8 substituent is a cyclic amine moiety the purine can be prepared from the 6-aminopurine by reaction with an appropriate dialkylating agent such as dihaloalkane. In some cases where the 6-substituent is a nitrogen containing heteroaromatic linked through the nitrogen atom the purine may be prepared from the 6-aminopurine by reaction with a dicarbonyl compound or a reactive derivative of this such as an acetal. For example 6-(1H-pyrrol-1-yl)-1H-purine can be prepared from a 6-chloropurine by reaction with 2,5-dimethoxytetrahydrofuran as described by Estep et al J Med Chem 1995, 38, 2582.

D. General Synthesis of 6-aryl(heteroaryl)/alkyl-substituted purine and 4-aryl(heteroaryl)/alkyl-substituted pyrimidine

Synthesis of 6-aryl(heteroaryl)/alkyl-substituted purines and 4-aryl(heteroaryl)/alkyl-substituted pyrimidines is shown in Scheme 2.

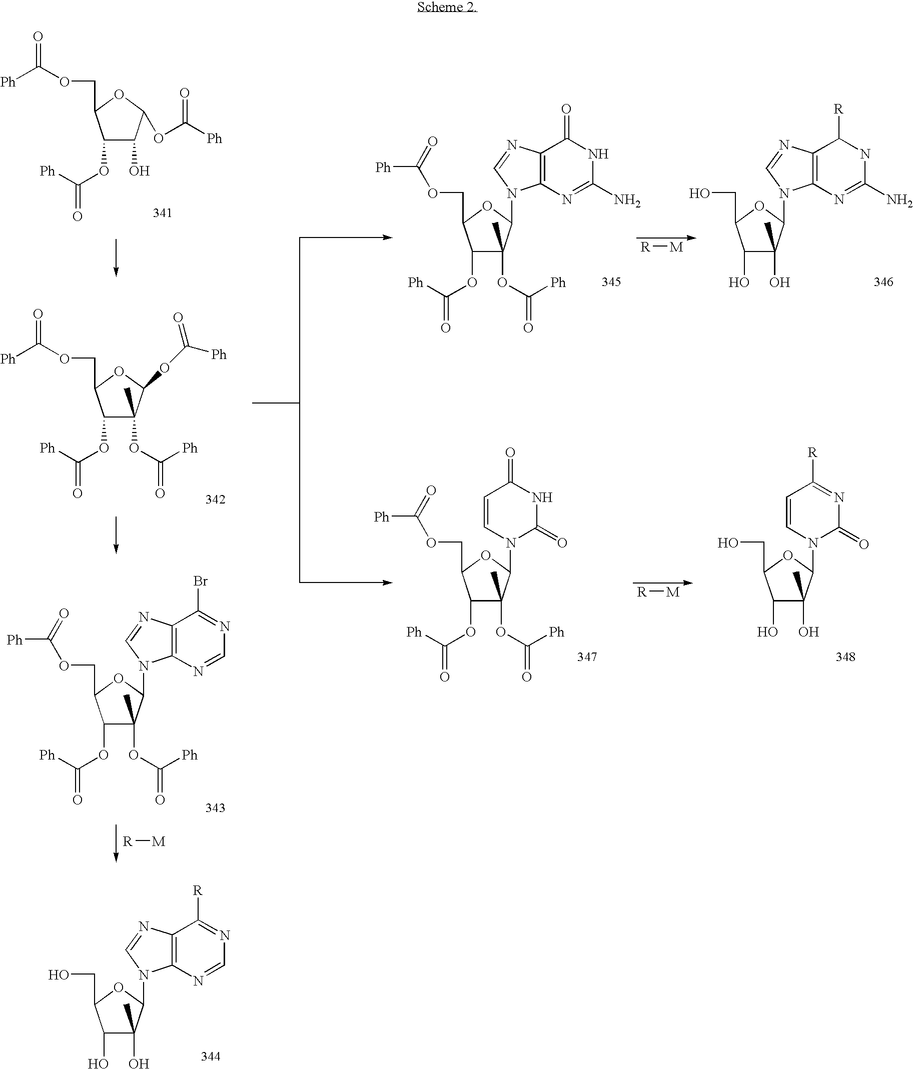

Commercial 341 is converted to the 2′meth-ribose derivative 342 as described in Wolfe, et al., J. Org. Chem., 1997, 62, 1754. 6-Bromopurine 2′-methylriboside (343) is prepared using the procedure for the synthesis of 6-chloropurine described in Wolfe, et al., J. Org. Chem., 1997, 62, 1754. 6-aromatic-substituted purine 2′-methylribosides 344 are synthesized using the protocols reported by Hocek et al., J. Med. Chem., 2000, 43, 1817 with commercially available boronic acids (R-M in Scheme 2). 6-alkyl-substituted purine 2′-methylribosides 344 are synthesized using modifications of the protocol reported by Bergstrom and Reday, Tet. Lett., 1982, 23, 4191. 6-aromatic-substituted-2-amino-purine 2′-methylribosides 345 are synthesized using modification of the protocols reported by Lakshman et al., Org. Lett.., 2002, 4, 1479 with commercially available boronic acids (R—B(OH)₂ in Scheme 2). 6-alkyl-substituted-2-amino-purine 2′-methylribosides 345 are synthesized using modifications of the protocol reported by Bergstrom and Reday, Tet. Lett., 1982, 23, 4191.

In similar manner, but using the appropriate pyrimidine bases, 4-aryl(heteroaryl)/alkyl-substituted pyrimidines 348 are synthesized.

According to this protocol, the following nucleosides are prepared.

| # | Structure | Name |
|---|---|---|
| 1 | 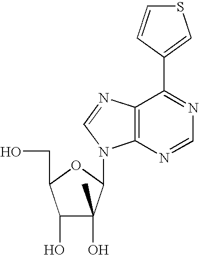 | 9-(2′-C-methyl-β-D- ribofuranosyl)-6-(thiophen- 3-yl)-purine |
| 2 | 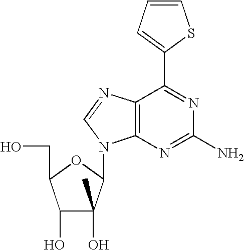 | 9-(2′-C-methyl-β-D- ribofuranosyl)-6-(thiophen- 2-yl)-2-aminopurine |
| 3 | 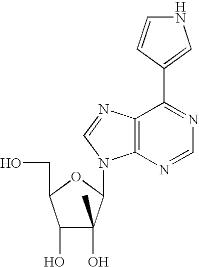 | 9-(2′-C-methyl-β-D- ribofuranosyl)-(pyrrol-3- yl)-purine |
| 4 | 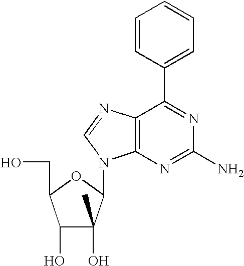 | 9-(2′-C-methyl-β-D- ribofuranosyl)-6-phenyl-2- aminopurine |
| 5 | 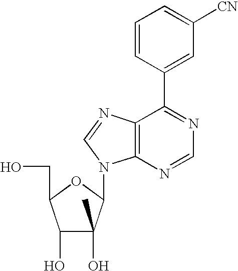 | 9-(2′-C-methyl-β-D- ribofuranosyl)-6-(3- cyanophenyl)-purine |
| 6 | 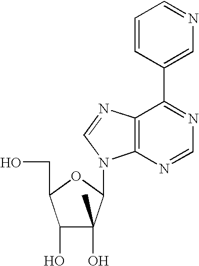 | 9-(2′-C-methyl-β-D- ribofuranosyl)-6-(pyridin- 3-yl)-purine |
| 7 | 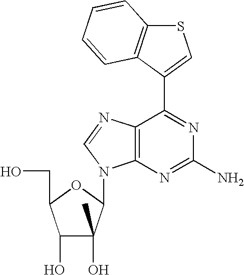 | 9-(2′-C-methyl-β-D- ribofuranosyl)-6- (Benzo[b]thiophen-3-yl)-2- aminopurine |
| 8 | 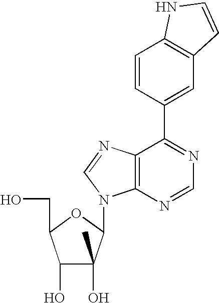 | 9-(2′-C-methyl- -D- ribofuranosyl)-6-(1H-Indol- 5-yl)-purine |
| 9 | 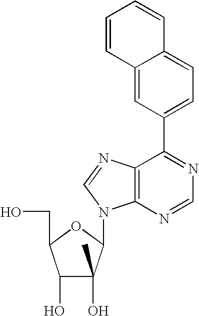 | 9-(2′-C-methyl- ® -D- ribofuranosyl)-6- (naphthalen-2-yl)-purine |
| 10 | 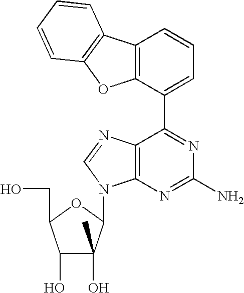 | 9-(2′-C-methyl-β-D- ribofuranosyl)-6- (dibenzofuran-4-yl)-2- aminopurine |
| 11 | 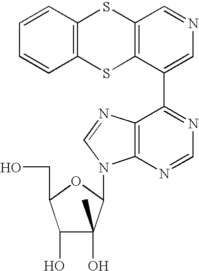 | 9-(2′-C-methyl-β-D- ribofuranosyl)-6- (thianthren-1-yl)-purine |
| 13 | 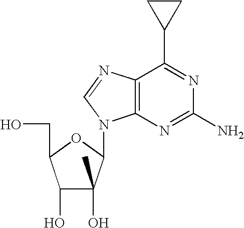 | 9-(2′-C-methyl-β-D- ribofuranosyl)-6- cyclopropyl-2-aminopurine |
| 14 | 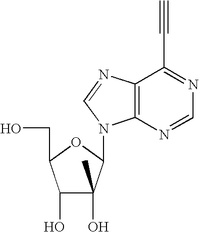 | 9-(2′-C-methyl-β-D- ribofuranosyl)-6-(ethynyl)- purine |
| 15 | 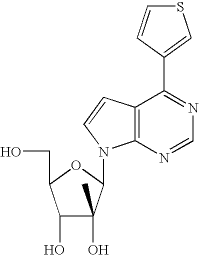 | 7-(2′-C-methyl-β-D- ribofuranosyl)-4-thiophen- 3-yl-7H-pyrrolo[2,3- d]pyrimidine |
| 16 | 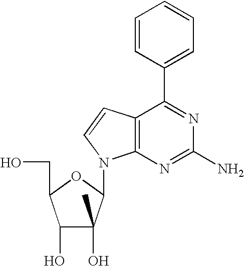 | 7-(2′-C-methyl-β-D- ribofuranosyl)-4-phenyl- 7H-pyrrolo[2,3- d]pyrimidin-2-ylamine |
| 17 | 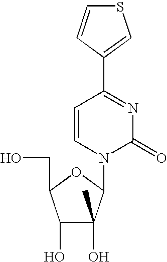 | 1-(2′-C-methyl-β-D- ribofuranosyl)-4-thiophen- 3-yl-1H-pyrimidin-2-one |
| 18 | 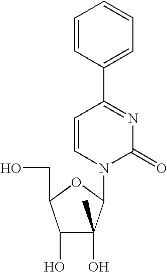 | 1-(2′-C-methyl-β-D- ribofuranosyl)-4-phenyl- 1H-pyrimidin-2-one |
| 19 | 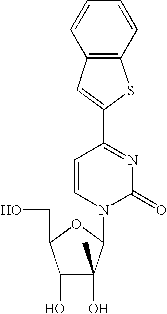 | 1-(2′-C-Methyl-β-D- ribofuranosyl)-4- benzo[b]thiophen-2-yl- 1H-pyrimidin-2-one |
| 21 | 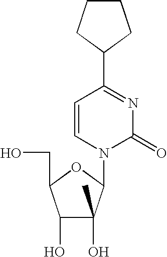 | 1-(2′-C-methyl-β-D- ribofuranosyl)-4- cyclopentyl-1H-pyrimidin- 2-one |

E. General Synthesis of N6-substituted adenine and N4-substituted cytosine

Synthesis of 6-aryl(heteroaryl)/alkyl-substituted purines and 4-aryl(heteroaryl)/alkyl-substituted pyrimidines is shown in Scheme 3.

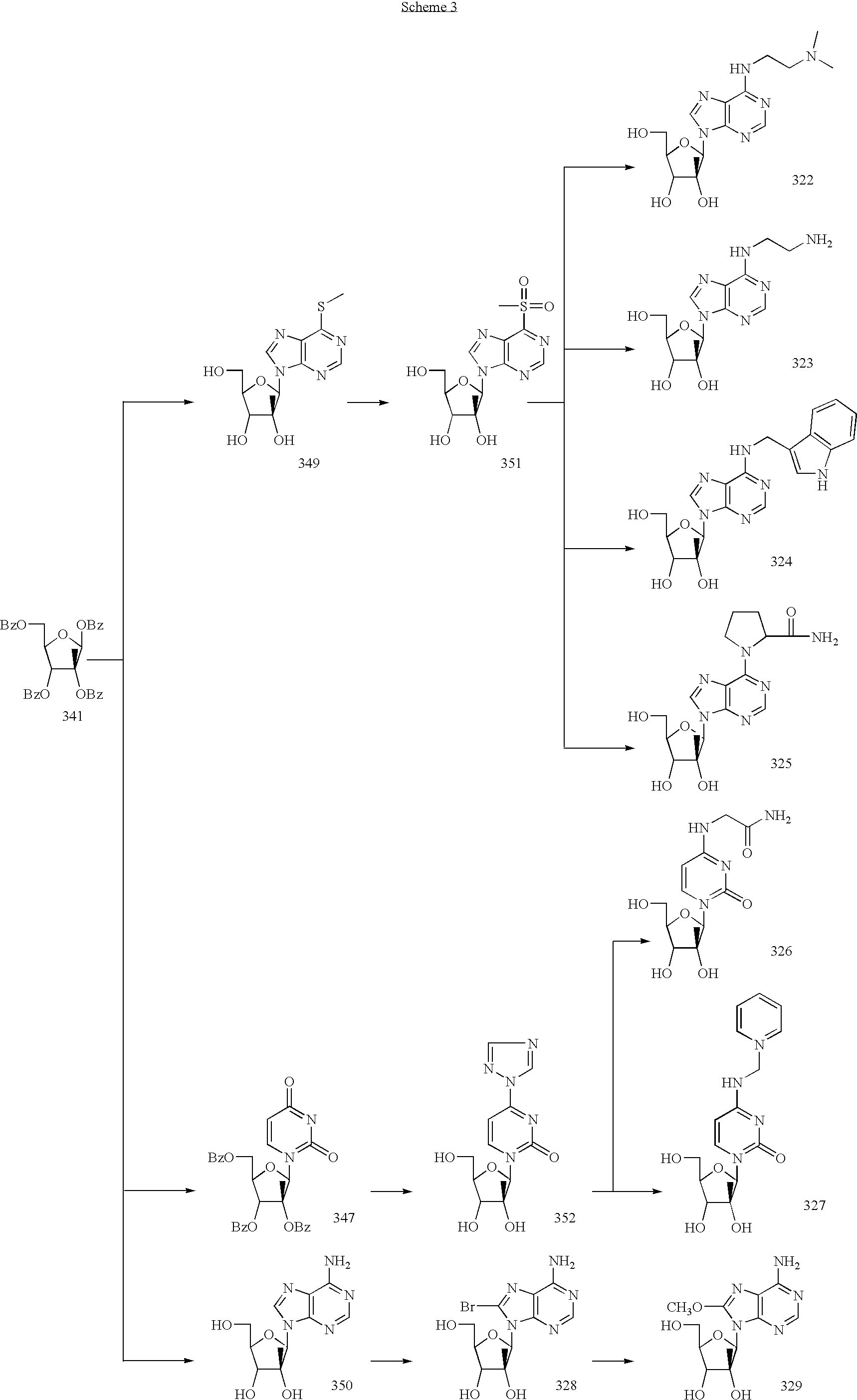

Synthesis of 9-(2′-C-methyl-β-D-ribofuranosyl)-6-methylthio-purine 49, 9-(2′-C-methyl-β-D-ribofuranosyl)-uridine 347, and 9-(2′-C-methyl-β-D-ribofuranosyl)-6-methylthio-adenine 350 are performed as described by R. Harry-O'kuru, J. Smith, and M. Wolf J. Org. Chem. 1997, 62, 1754-1759. Methylthio-purine is oxidized to methylsulfonyl-purine using the procedure described by Y -Z. Xu Tetrahedron, 1996, 52, 10737-10750; Y -Z. Xu, Q. Zheng, and P. Swann Nucleosides Nucleotides 1995, 14, 929-934. For substitution of methylsulfonyl and triazolyl groups for amine, protocols similar to the protocol reported for deoxynucleosides by P.Srivastava, G.Revankar, R.Robins, and R.Rousseau J. Med. 10 Chem, 1981, 24, 393-398, can be used. Synthesis of 4-triazolyl-uridine and it substitution with amines can be performed as described for 2′-deoxythymidine by Y. -Z. Xu, Q. Zheng, and P. Swann J. Org. Chem. 1992, 57, 3839-3845. Bromination of purine nucleosides can be performed as described by J.Gerster et al. J. Org. Chem. 1968, 33, 1070-1073.

| # | Structure | Name |
|---|---|---|
| 22 | 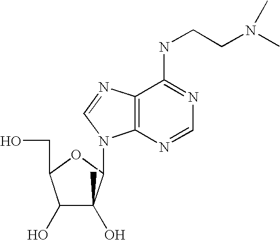 | 9-(2′-C-methyl-β-D-ribofuranosyl)- N⁶-(2-dimethylaminoethyl)-adenine |
| 23 | 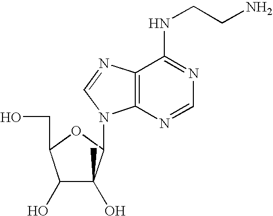 | 9-(2′-C-methyl-β-D-ribofuranosyl)-N⁶- (2-aminoethyl)adenine |
| 24 | 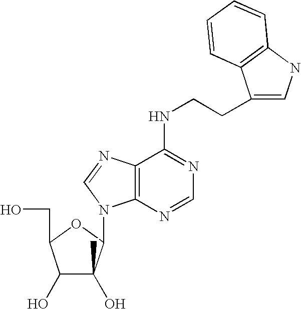 | 9-(2′-C-methyl-β-D-ribofuranosyl)-N⁶- [2-(3H-indol-3-yl)-ethyl]adenine |
| 25 | 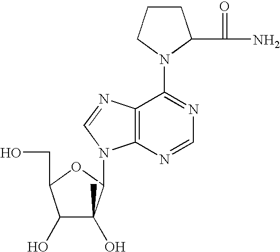 | 9-(2′-C-methyl-β-D-ribofuranosyl)-6- [2-aminocarbonyl-(pyrrolidine-1-yl)]- purine |
| 26 | 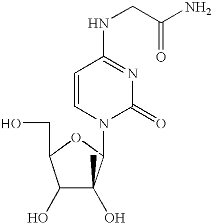 | 1-(2′-C-methyl-β-D-ribofuranosyl)- N⁴-(aminocarbonylmethyl)cytidine |
| 27 | 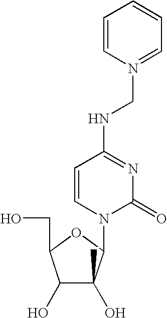 | 1-(2′-C-methyl-β-D-ribofuranosyl)- N⁴-[(pyridin-1-yl)-methyl]cytidine |
| 30 | 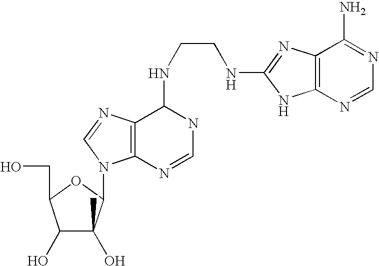 | 9-(2′-C-methyl-β-D-ribofuranosyl)-N⁶- [(adenin-8-yl)-aminoethyl]adenine |
| 31 | 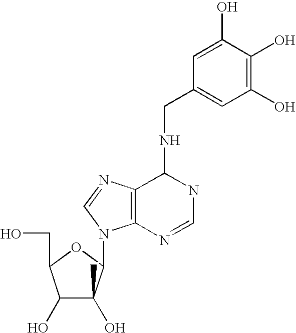 | 9-(2′-C-methyl-β-D-ribofuranosyl)-N⁶- [(benzene-3,4,5-triol)methyl]adenine |
| 32 | 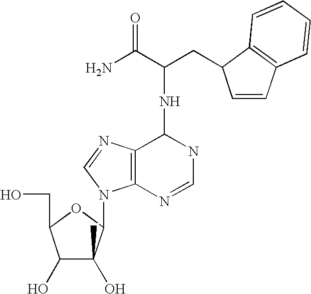 | 9-(2′-C-methyl-β-D-ribofuranosyl)-N⁶- [1-aminocarbonyl-2-(3H-indol-3-yl)- ethyl]adenine |
| 33 | 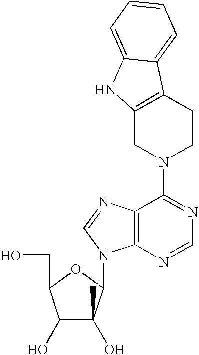 | 9-(2′-C-methyl-β-D-ribofuranosyl)-6- (1,3,4,9-tetrahydro-beta-carbolin-2- yl)purine |
| 34 | 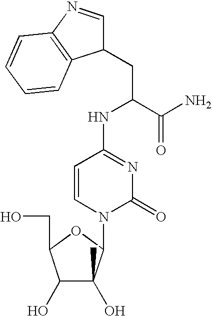 | 1-(2′-C-methyl-β-D-ribofuranosyl)- N⁴-[1 -aminocarbonyl-2-(3H-indol-3- yl)-ethyl]cytosine |
| 35 | 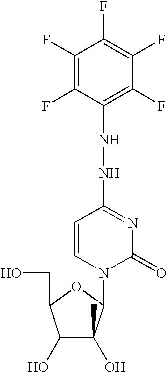 | 1-(2′-C-methyl-β-D-ribofuranosyl)-4- (pentafluorophenyl-hydrazino)- pyrimidin-2-one |
| 37 | 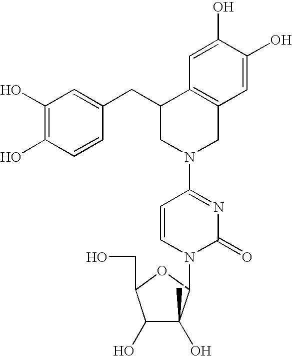 | 1-(2′-C-methyl-β-D-ribofuranosyl)-4- [4-(3,4-dixydroxy-benzyl)-6,7- dihyrdoxy-3,4-dihydro-1H-isoquinolin- 2-yl]-pyrimidin-2-one |
| 38 | 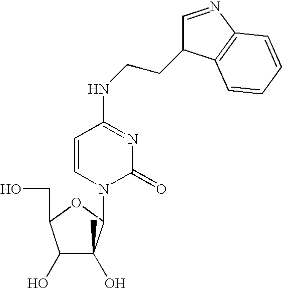 | 1-(2′-C-methyl-β-D-ribofuranosyl)- N⁴-[2-(3H-indol-3-yl)-ethyl]cytosine |
| 39 | 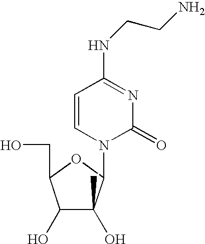 | 1-(2′-C-methyl-β-D-ribofuranosyl)- N⁴-(2-aminoethyl)cytosine |
| 40 | 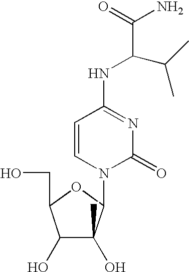 | 1-(2′-C-methyl-β-D-ribofuranosyl)- N⁴-(aminocarbonyl-isopropyl- methyl)cytidine |
| 53 | 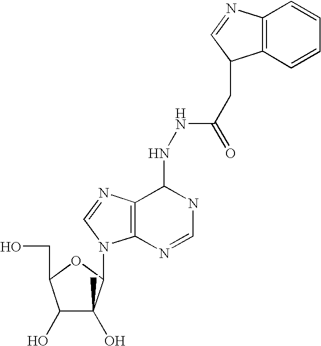 | 9-(2′-C-methyl-β-D-ribofuranosyl)-N⁶- {[(3H-indol-3-yl)-acetic acid]- hydrazide}adenine |
| 54 | 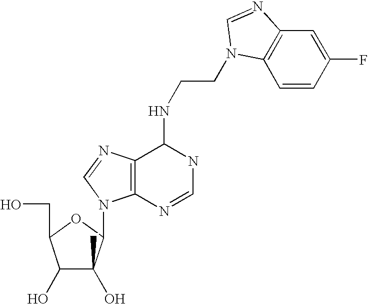 | 9-(2′-C-methyl-β-D-ribofuranosyl)-N⁶- [2-(5-fluoro-benzimidazol-1-yl)- ethyl]adenine |
| 55 | 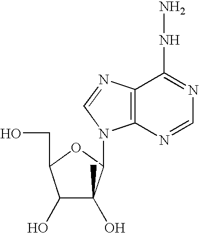 | 9-(2′-C-methyl-β-D-ribofuranosyl)-6- hydrazino-purine |
| 56 | 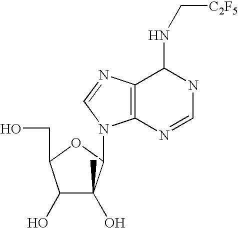 | 9-(2′-C-methyl-β-D-ribofuranosyl)-N⁶- (2,2,3,3,3,-pentafluoropropyl)adenine |
| 57 | 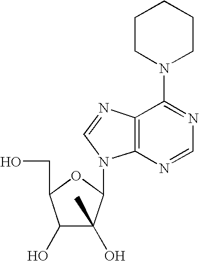 | 9-(2′-C-methyl-β-D-ribofuranosyl)-6- (piperidin-1-yl)purine |
| 106 | 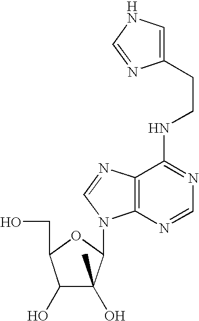 | 9-(2′-C-methyl-β-D-ribofuranosyl)-6- [2-(1H-imidazol-4-yl)-ethyl]purine |
| 107 | 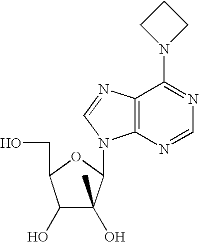 | 9-(2′-C-methyl-β-D-ribofuranosyl)-6- (azetidin-1-yl)purine |
| 108 | 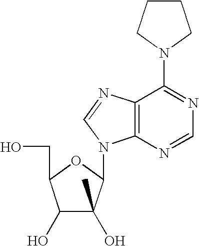 | 9-(2′-C-methyl-β-D-ribofuranosyl)-6- (pyrrolidin-1-yl)purine |
| 110 | 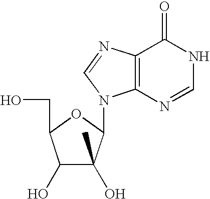 | (2′-C-methyl-β-D-ribofuranosyl)- hypoxanthine |
| 112 | 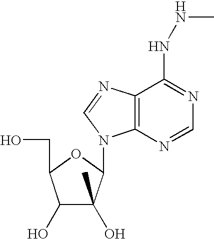 | 9-(2′-C-methyl-β-D-ribofuranosyl)-6- methylhydrazinopurine |
| 113 | 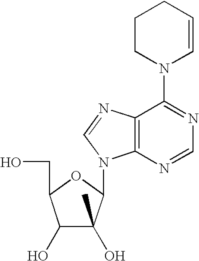 | 9-(2′-C-methyl-β-D-ribofuranosyl)-6- (3,6-dihydro-2H-pyridin-1-yl)purine |
| 114 | 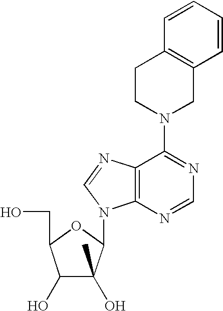 | 9-(2′-C-methyl-β-D-ribofuranosyl)-6- (3,4-dihydro-1H-isoquinolin-2- yl)purine |

Following procedures set forth above and procedures well-known in the art, as well as those described by Li et al.³⁵, 2′-C-trifluoromethyl-β-D-ribofuranosyl derivatives can be prepared.

By following the procedures set forth above, as well as procedures well known in the art, including those procedures set forth by Devos⁴, et al. and Sommadossi⁵ et al., the following compounds can be made.

1-Deazapurines can be prepared and coupled to ribofuranosyl derivatives as described in by Cristalli, et al. in J. Med. Chem., 1987, 30(9) p. 1686 or Seela, F., et al. in Nucleosides Nucleotides, 1998, 17(4), p. 729.

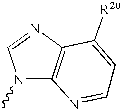

Purine nucleosides can be prepared and coupled to ribofuranosyl derivatives using methods and materials described herein.

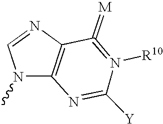

Benzimidazole nucleosides can be prepared and coupled to ribofuranosyl derivatives as described in by Sagi, G., et al., in J. Med. Chem. 1992, 35(24), 4549.

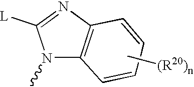

5-Pyrrolopyridine Nucleosides can be prepared and coupled to ribofuranosyl derivatives as described in Tetrahedron 1976, 32, 773.

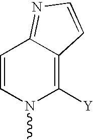

4-Pyrimidopyridone Sangivamycin Analogs can be prepared and coupled to ribofuranosyl derivatives as described in J. Org. Chem., 1972, 37, 3980, and J. Org. Chem., 1977, 42, 997.

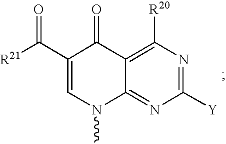

2-Pyrimidopyridone Sangivamycin Analogs can be prepared and coupled to ribofuranosyl derivatives as described in J. Org. Chem., 1977, 42, 997.

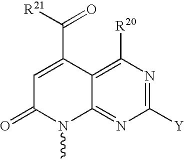

4-Pyrimidopyridone Sangivamycin Analogs can be prepared and coupled to ribofuranosyl derivatives as described in J. Org. Chem., 1972, 37, 3975.

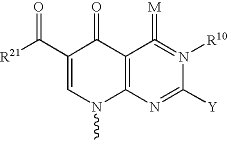

Pyrimidopyridine Analogs can be prepared and coupled to the sugar as described in Chem. Pharm. Bull., 1968, 16, 1076, and J. Org. Chem., 1972, 37, 3975.

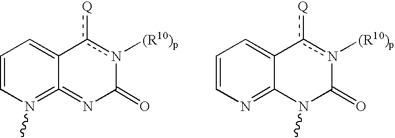

Pyrimido-tetrahydropyridines can be prepared and coupled to ribofuranosyl derivatives as described in Biorog. Khim., 1979, 5, 1369.

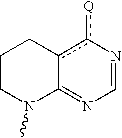

Furanopyrimidines (& tetrahydro furanopyrimidines) can be prepared and coupled to ribofuranosyl derivatives as described in J. Med. Chem., 1983, 26, 661; J. Org. Chem., 1983, 48, 1854; and J. Med. Chem., 1985, 28, 1679.

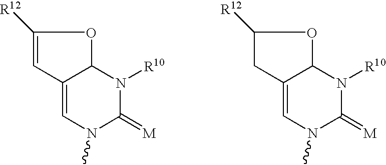

Pyrazolopyrimidines can be prepared and coupled to ribofuranosyl derivatives as described in Chem. Ber., 1981, 114, 1610, and J. Med. Chem., 1983, 26, 1601.

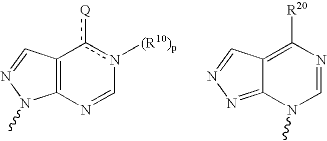

Pyrolopyrimidines can be prepared and coupled to ribofuranosyl derivatives as described in Liebigs Ann. Chem., 1983, 1576.

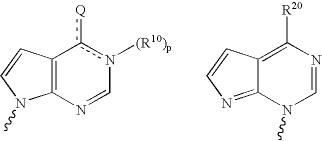

Triazolopyrimidines can be prepared and coupled to ribofuranosyl derivatives as described in J. Heterocycl. Chem., 1971, 8, 237, and J. Carbohydr. Nucleosides Nucleotides, 1976, 3, 281.

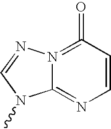

Pteridines can be prepared and coupled to ribofuranosyl derivatives as described in Nucleosides Nucleotides, 1989, 8, 1345, and Chem. Berich., 1974, 107, 3377.

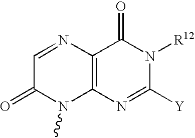

Pyridine C-nucleosides can be prepared by coupling ribofuranosyl derivatives to a variety of bases as described in Angew. Chem. Int. Ed. Engl., 1996, 35, 1968, and Helv. Chim. Acta, 1996, 79, 702-709.

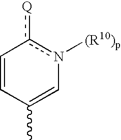

Pyrazolotriazine C-nucleosides can be prepared by coupling ribofuranosyl derivatives to a variety of bases as described in J. Heterocycl. Chem., 1976, 13, 175; J Heterocycl. Chem., 1976,13, 1305; J. Heterocycl. Chem., 1980, 17, 1435; J. Org. Chem., 1977, 42, 109.

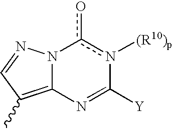

9-Deazapurine C-nucleosides can be prepared by coupling ribofuranosyl derivatives to a variety of bases as described in J. Org. Chem., 1977, 42, 109; Chem. Ber., 1968, 101, 41; Tet. Lett., 1981, 21, 1013; J. Org. Chem., 1967, 32, 1825; J. Heterocycl. Chem., 1978, 15, 353; Tet. Lett., 1981, 22, 25; Tet. Lett., 1986, 27, 815; and J. Med. Chem., 1990, 33, 2750.

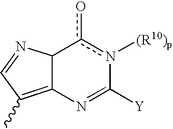

Indole nucleosides can be prepared by coupling ribofuranosyl derivatives to a variety of indole bases as described in Yokoyama, M., et al., J. Chem. Soc. Perkin Trans. I, 1996, 2145.

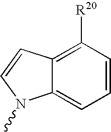

Utility, Testing, and Administration

Utility

The present invention provides novel compounds possessing antiviral activity, including hepatitis C virus. The compounds of this invention inhibit HCV replication by inhibiting the enzymes involved in replication, including RNA dependent RNA polymerase. They may also inhibit other enzymes utilized in the activity or proliferation of HCV.

The compounds of the present invention can also be used as prodrug nucleosides. As such they are taken up into the cells and can be intracellularly phosphorylated by kinases to the triphosphate and are then inhibitors of the polymerase (NS5b) and/or act as chain-terminators.

Compounds of this invention maybe used alone or in combination with other compounds to treat viruses.

Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of Formula Ia, Ib, Ic, II, IIA, III, or IV may range from approximately 0.05 to 50 mg per kilogram body weight of the recipient per day; preferably about 0.01-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract, in particular for the treatment of diseases such as asthma and similar or related respiratory tract disorders (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula Ia, Ib, Ic, II, IIA, III, or IV in combination with at least one pharmaceutically acceptable excipient. Acceptable excipients are non-toxic, aid administration, and do not adversely affect the therapeutic benefit of the compound of Formula Ia, Ib, Ic, II, IIA, III, or IV. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula Ia, Ib, Ic, II, IIA, III, or IV based on the total formulation, with the balance being one or more suitable pharmaceutical excipients. Preferably, the compound is present at a level of about 1-80 wt %. Representative pharmaceutical formulations containing a compound of Formula Ia, Ib, Ic, II, IIA, III, or IV are described below.

### EXAMPLES

In the examples below, the following abbreviations have the following meanings. If an abbreviation is not defined, it has its generally accepted meaning.

| | |
|---|---|
| % mol = | mol percent |
| AcOEt = | ethylacetate |
| μL = | microliters |
| Arg = | arginine amino acid residue |
| Boc Py = | N-Boc-4-amino-1-methyl pyrrole-2-carboxy- |
| | lic acid |
| Boc = | t-butoxycarbonyl |
| Boc-5-Ain = | N-Boc-5-Amino-Indole-2-Carboxylic Acid |
| Boc-5-Ain-HBA-AMPS = | N-Boc-5-Amino-Indole-2-Carboxylic Acid |
| | (p-Hydroxy benzamide methyl polystyrene) |
| | ester |
| Boc-Py-HBA-AMPS = | N-Boc-4-Amino-1-Methyl Pyrrole-2-Car- |
| | boxylic Acid (p-Hydroxy benzamide methyl |
| | polystyrene)ester |
| BOP = | Benzotriazol-1-yloxy- |
| | tris(dimthylamino)phosphonium |
| | hexafluorophosphate |
| brd = | broad doublet |
| brm = | broad multiplet |
| brt = | broad triplet |
| bs = | broad singlet |
| Bzl = | benzyl protecting group |
| conc. = | concentrated |
| dba = | dibenzyledene acetone |
| DCC = | dicyclohexylcarbodiimide |
| DCE = | 1,2-dichloroethane |
| DCM = | dichloromethane |
| DCU = | N,N′-dicyclohexylurea |
| dd = | doublet of doublets |
| DE = | 2-(Dimethylamino)ethylamine |
| DIAD = | diisopropyl azo dicarboxylate |
| DIC = | N,N′ diisopropyl carbodiimide |
| DIPEA = | diisopropylethylamine |
| DMAP = | 4-N,N-dimethylaminopyridine |
| DME = | dimethoxyethane |
| DMF = | N,N-dimethylformamide |
| DMSO = | dimethylsulfoxide |
| DP = | 3-(Dimethylamino)propylamine |
| DPPA = | diphenylphosphoryl azide |
| dppf = | 1,1′-bis(diphenylphosphino)ferrocene |
| dt = | doublet of triplets |
| eq. = | equivalents |
| Et = | ethyl radical |
| EtOH = | ethanol |
| Fmoc = | fluorenylmethoxycarbonyl protecting group |
| g = | gram |
| Gly for a; = | glycine amino acid residue |
| h = | hours |
| HBA-AMPS = | p-hydroxybenzamide -methylpolystyrene |
| HBTU = | O-Benzotriazol-1-yl-N,N,N′,N′- |
| | tetramethyluronium hexafluorophosphate |
| HPLC = | high performance liquid chromatography |
| LC/MS = | liquid chromatography/mass spectroscopy |
| Lys = | lysine amino acid residue |
| M = | molar |
| mM = | millimolar |
| m = | multiplet |
| Me f = | methyl radical |
| MeOH = | methanol |
| mg = | milligram |
| min. = | minutes |
| mL = | milliliter |
| mm = | millimeter |
| mmol = millimole | |
| MMT = | monomethoxytrytil (p-anisyldiphenylmethyl) |
| | protecting group |
| mp = | melting point |
| mp d = | melting point with decomposition |
| MS for; = | mass spectrum |
| M = | normal |
| NMR = | nuclear magnetic resonance spectrum |
| Np = | 4-nitophenyl radical |
| Npc(Et) = | 4-nitro-1-ethyl-1H-pyrrole-2-carboxylic acid |
| | residue |
| Npc(Me) = | 4-nitro-1-methyl-1H-pyrrole-2-carboxylic |
| | acid residue |
| Npc(Pr) = | 4-nitro-1-propyl-1H-pyrrole-2-carboxylic |
| | acid residue |
| Pfp = | pentafluorophenyl radical |
| Phe = | phenyl radical |
| psi = | pounds per square inch |
| Py = | 4-amino-1-methyl-1H-pyrrole-2-carboxylic |
| | acid residue |
| Pyr = | pyridine |
| Pzl-Gu-(Boc)₂ = | N,N′-Bis(tert-butoxycarbonyl)-1H-pyrazole- |
| | 1-carboxamidine |
| q = | quartet |
| rpm = | rotations per minute |
| Rₜ = | retention time |
| rt = | room temperature |
| s = | singlet |
| t = | triplet |
| t-Bu = | t-butyl protecting group |
| TEA = | triethylamine |
| TFA = | trifluoroacetic acid |
| THF = | tetrahydrofuran |
| TLC = | thin layer chromatography |
| Z = | benzyloxycarbonyl protecting group |
| v/v = | volume/volume |
| v/v/v = | volume/volume/volume |
| BSA = | bis-trimethylsilylacetamide |
| TMSOTf = | tri-methylsilyl trifluoromethan sulfonate |
| nm = | nanometer |
| RP HPLC = | reverse phase HPLC |
| MBS = | N-bromosuccinimide |
| NIS = | N-iodosuccinimide |
| DI = | deionized |
| NMP = | N-methylpyrrolidone |
| PPA = | polyphosphoric acid |
| Hex = | hexane |
| DMEM = | Dulbeco's Modified Eagle's Medium |

In reporting NMR data, chemical shifts are given in ppm and coupling constants (J) given in Hertz (Hz). All melting points are uncorrected.

In the following examples and procedures, the starting amterials and regeants are commercially available from any one of Aldrich, Lancaster, Sigma, Specs, TCI, Maybridge Frontier Scientific and Bachem. The term “Aldrich” indicates that the compound or reagent used in the procedure is commercially available from Aldrich Chemical Company, Inc., Milwaukee, Wis. 53233 USA; the term “Lancaster” indicates that the compound or reagent is commercially available from Lancaster Synthesis, Inc., NH 03087 USA; the term “Sigma” indicates that the compound or reagent is commercially available from Sigma, St. Louis Mo. 63178 USA; the term “Maybridge” indicates that the compound or reagent is commercially available from Maybridge Chemical Co. Trevillett, Tintagel, Cornwall PL34 OHW United Kingdom; and the term “TCI” indicates that the compound or reagent is commercially available from TCI America, Portland Oreg. 97203; the term “Frontier Scientific” indicates that the compound or reagent is commercially available from Frontier Scientific, Utah, USA; the term “Specs” indicates that the compound or reagent is commercially available from Netherlands; and “Bachem” indicates that the compound or reagent is commercially available from Bachem, Torrance, Calif., USA.

Set forth in the examples below are compounds and intermiediates useful for making compounds of the present invention.

### Example 1

Synthesis of 9-(2′-C-methyl-β-D-ribofuranosyl)-6-bromopurine (41)

9-(2′-C-methyl-β-D-ribofuranosyl)-6-bromopurine (41) can be synthesized utilizing the general procedure described in R. Harry-O'kuru, J. Smith, and M. Wolf J. Org. Chem. 1997, 62, 1754-1759.

### Example 2

Synthesis of 9-(2′-C-methyl-β-D-ribofuranosyl)-6-(thiophen-3-yl)-purine (1)

Toluene (10 mL) is added to an argon-purged flask containing 9-(2′-C-methyl-β-D-ribofuranosyl)-6-bromopurine (41) (1 mmol), K₂CO₃ (200 mg, 1.5 mmol), 3-thiopheneboronic acid (1.5 mmol) and Pd(PPh₃)₄ (59 mg, 0.05 mmol) and the mixture is stirred under argon at 100° C. for 8 h. After cooling to ambient temperature the mixture is evaporated in vacuo and the residue is chromatographed on a silica gel column. The residue is then taken up into 10 mL NH₃ saturated MeOH and reacted at 55° C. for 12 hours in a sealed tube. The reaction was cooled and concentrated in vacuo. The product was isolated by column chromatography on silica gel (chloroform/methanol/ammonia 9:1:0.5 v/v/v).

### Example 3

Synthesis of 9-(2′-C-methyl-β-D-ribofuranosyl)-N²-isobutyryl-guanosine (42)

9-(2′-C-methyl-β-D-ribofuranosyl)-N²-isobutyryl-guanosine (42) is synthesized utilizing the general procedure described in R. Harry-O'kuru, J. Smith, and M. Wolf J. Org. Chem. 1997, 62, 1754-1759 and is isolated by HPLC.

### Example 4

Synthesis of 9-(2′-C-methyl-β-D-ribofuranosyl)-2-amino-6-phenylpurine (4)

9-(2′-C-methyl-β-D-ribofuranosyl)-N²-isobutyryl-guanosine (42) (1 mmol) is dissolved in dichloromethane (10 mL) under argon and 2,6-di-tert.butyl-4-methylpyridine (3 mmol) is added. The solution is cooled to 0 ° C. and trifluoromethanesulfonic anhydride (3 mmol) is added and the reaction is allowed to warm to ambient temperature. After 12 hours the reaction is concentrated in vacuo and chromatographed on silica gel (ethyl acetate/dichoromethane). The product is dissolved in toluene (10 mL) and then K₂CO₃ (200 mg, 1.5 mmol), phenylboronic acid (1.5 mmol) and Pd(PPh₃)₄ (59 mg, 0.05 mmol) are added and the mixture is stirred under argon at 100° C. for 8 h. After cooling to ambient temperature the mixture is evaporated in vacuo and the residue is chromatographed on a silica gel column. The residue is then taken up into 10 mL NH₃ saturated MeOH and reacted at 55° C. for 12 hours in a sealed tube. The reaction is cooled and concentrated in vacuo. The product is isolated by column chromatography on silica gel (chloroform/methanol/ammonia 9:1:0.5 v/v/v).

### Example 5

Synthesis of 9-(2′-C-methyl-β-D-ribofuranosyl)-uracil (43)

9-(2′-C-methyl-β-D-ribofuranosyl)-uracil (43) is synthesized as described in R. Harry-O'kuru, J. Smith, and M. Wolf i J. Org. Chem. 1997, 62, 1754-1759.

### Example 6

Synthesis of 1-(2′-C-methyl-β-D-ribofuranosyl)-4-thiophen-3-yl-1H-pyrimidin-2-one (17)

9-(2′-C-methyl-β-D-ribofuranosyl)-uracil (43) (1 mmol) is dissolved in dichloromethane (10 mL) under argon and 2,6-di-tert.butyl-4-methylpyridine (3 mmol) is added. The solution is cooled to 0° C. and trifluoromethanesulfonic anhydride (3 mmol) is added and the reaction is allowed to warm to ambient temperature. After 12 hours the reaction is concentrated in vacuo and chromatographed on silica gel (ethyl acetate/dichoromethane). The product is dissolved in toluene (10 mL) and then K₂CO₃ (200 mg, 1.5 mmol), 3-thiopheneboronic acid (1.5 mmol) and Pd(PPh₃)₄ (59 mg, 0.05 mmol) are added and the mixture is stirred under argon at 100° C. for 8 h. After cooling to ambient temperature the mixture is evaporated in vacuo and the residue is chromatographed on a silica gel column. The residue is taken up into 10 mL NH₃ saturated MeOH and is reacted at 55° C. for 12 hours in a sealed tube. The reaction is cooled and concentrated in vacuo. The product is isolated by column chromatography on silica gel (chloroform/methanol/ammonia 9:1:0.5 v/v/v).

### Example 7

Synthesis of 1-(2′-C-methyl-β-D-ribofuranosyl)-4-cyclopentyl-1H-pyrimidin-2-one (21)

9-(2′-C-methyl-β-D-ribofuranosyl)-uracil (43) (1 mmol) is dissolved in dichloromethane (10 mL) under argon and 2,6-di-tert.butyl-4-methylpyridine (3 mmol) is added. The solution is cooled to 0° C. and trifluoromethanesulfonic anhydride (3 mmol) is added and the reaction is allowed to warm to ambient temperature. After 12 hours the reaction is concentrated in vacuo and chromatographed on silica gel (ethyl acetate/dichoromethane). The product is dissolved in anhydrous THF (10 mL) and Pd(PPh₃)₄ (59 mg, 0.05 mmol) is added under Ar atmosphere. Cyclopentylzinc bromide (1.5 mmol, 0.5 M in THF) is then added and the reaction stirred at ambient temperature for 18 hours. The mixture is evaporated in vacuo and the residue is chromatographed on a silica gel column. The residue is taken up into 10 mL NH3 saturated MeOH and reacted at 55° C. for 12 hours in a sealed tube. The reaction is cooled and concentrated in vacuo. The product is isolated by column chromatography on silica gel (chloroform/methanol/ammonia 9:1:0.5 v/v/v).

### Example 8

Synthesis of 9-(2′-C-methyl-β-D-ribofuranosyl)-6-methylthio-purine (49)

9-(2′-C-methyl-β-D-ribofuranosyl)-6-methylthio-purine (49) is synthesized as described in R. Harry-O'kuru, J. Smith, and M. Wolf J. Org. Chem. 1997, 62, 1754-1759.

### Example 10

Synthesis of 9-(2′-C-methyl-β-D-ribofuranosyl)-6-[2-(1H-imidazol-4-yl)-ethyl]purine (106).

Compound 106 was synthesized from histamine and nucleoside 51 as described in Example 9, step 4.

MS 361.45 (M+H)

H¹-NMR (DMSO-d6): 0.80 (s, 3H, 2′-CH₃), 3.25-3.45 (m, 4H, methylene), 3.53-4.05 (m, 7H, sugar), 5.99 (s, 1H, 1′-H), 7.48 and 9.09 (s, 1H, purine), 8.35 and 8.65 (bs, 0.7H, imidazole)

### Example 11

Synthesis of 9-(2′-C-methyl-β-D-ribofuranosyl)-N⁶-(2-aminoethyl)adenine (23)

Nucleoside (51) (1 mmol) is dissolved in pyridine (5 mL), ethylenediamine (5 mM) is added and the reaction mixture is kept overnight at room temperature. The solvent is evaporated; the product (23) is isolated by column chromatography on silica gel (chloroform/methanol/ammonia 9:1:0.5, v/v/v).

### Example 12

Synthesis of 9-(2′-C-methyl-β-D-ribofuranosyl)-6-[2-(1H-indol-3-yl) ethyl]purine (24).

Compound 24 was synthesized from tryptamine and nucleoside 51 as described in Example 9, step 4.

MS 410.38 (M+H)

H¹-NMR (DMSO-d6): 0.76 (s, 3H, 2′-CH₃), 2.60-4.10 (m, sugar and methylene), 5.98 (s, 1H, 1′-H), 6.80 (d, 1H, indole), 7.18 (m, 4H, indole), 8.35 and 8.68 (s, 1H, purine), 9.02 (s, 1H, NH).

### Example 13

Synthesis of 9-(2′-C-methyl-β-D-ribofuranosyl)-6-F(pyrrolidin-1-yl)-2-carboxamide]purine (25).

Compound 25 was synthesized from L-proline amide and nucleoside 51 as described in Example 9, step 4.

MS 380.35 (M+H)

H¹-NMR (DMSO-d6): 0.86 (s, 3H, 2′-CH₃), 2.25-3.95 (m, 4H, pyrrolidine), 3.10-4.10 (m, sugar and pyrrolidine), 5.98 (s, 1H, 1′-H), 8.35 and 8.68 (s, 1H, purine), 9.25 (s, 1H, amide).

### Example 14

Synthesis of 1-(2′,3′,5′-Tri-O-benzoyl -2′-C-methyl-β-D-ribofuranosyl)-uracil (47)

1-(2′,3′,5′-Tri-O-benzoyl-2′-C-methyl-β-D-ribofuranosyl)-uracil ( 47) is synthesized as described in R. Harry-O'kuru, J. Smith, and M. Wolf J. Org. Chem. 1997, 62, 1754-1759.

### Example 15

Synthesis of 1-(2′,3′,5′-Tri-O-benzoyl-2′-C-methyl-β-D-ribofuranosyl)-4-(1,2,4-triazol-1-yl) uracil (52)

1,2,4-Triazol (60 mmol) is suspended in dry acetonitrile (70 mL) at 0° C. Phosphorous oxychloride (15 mM) is slowly added with rapid stirring followed by drop wise addition of triethylamine (50 mmol). The reaction mixture is stirred for 30 min at 0° C. and than nucleoside (47) (15 mmol) is added. In 1 hour the reaction is quenched with 50 mL of saturated solution of sodium bicarbonate. The product is extracted with 50 mL of chloroform. Organic extract is washed with 5% sodium bicarbonate, water, dried over magnesium sulphate and evaporated. The product is isolated by column chromatography on silica gel (toluene/ethyl acetate).

### Example 16

Synthesis of 1-(2′-C-methyl-β-D-ribofuranosyl)-N⁴-(aminocarbonylmethyl)cytidine (26)

Nucleoside (52) (1 mmol) is dissolved in 95% pyridine (5 mL), glycine amide (5 mM) is added and the reaction mixture is kept for 16 hours at 55° C. The solvent is evaporated. The product (26) is isolated by column chromatography on silica gel (chloroform/methanol/ammonia 9:1:0.5 v/v/v).

### Example 17

Synthesis of 1-(2′-C-methyl-β-D-ribofuranosyl)-N⁴-(pyridin-1-ylmethyl)cytidine (27)

Nucleoside (52) (1 mmol) is dissolved in 95% pyridine (5 mL), pyridin-1-yl-methylamine (5 mM) is added and the reaction mixture is kept for 16 hours at 55° C. The solvent is evaporated. The product (27) is isolated by column chromatography on silica gel (chloroform/methanol/ammonia 9:1:0.5 v/v/v).

### Example 18

Synthesis of 2′-C-methyladenosine (50)

2′-C-methyladenosine (50) is prepared as described in R. Harry-O'kuru, J. Smith, and M. Wolf J. Org. Chem. 1997, 62, 1754-1759.

### Example 19

Synthesis of 2′-C-methyl-8-bromoadenosine (28)

Bromine (2 mL) is added to 50 mL of water and stirred vigorously at room temperature for 3 min. Nucleoside (50) (5 g) is suspended in 30 mL of water and Br₂-water is added by aliquots at such a rate that yellow color of the reaction mixture disappeared between each addition. The total amount of Br₂-water is 45 mL. The solid is collected by filtration and washed carefully with iced water up to pH 5.5. The residue is recrystallized from hot water to yield 60% of the target product.

### Example 21

Synthesis of 5-(2′-C-methyl-β-D-ribofuranosyl)-5H-pyrrolo[3,2-c]pyridin-4-ylamine (80)

The title compound can be prepared by methods similar to those set forth by Ducrocq⁶ on page 779 to 780.

### Example 22

Synthesis of 4-amino-8-(2′-C-methyl-β-D-ribofuranosyl)-5-oxo-5,8-dihydro-pyrido[2,3-d]pyrimidine-6-carboxylic acid amide (81)

The title compound can be prepared by methods similar to those set forth by Rizkalla⁷ on page 3985.

### Example 23

Synthesis of 2,4-Diamino-8-(2′-C-methyl-β-D-ribofuranosyl)-5-oxo-5,8-dihydro-pyrido[2.3-d]pyrimidine-6-carboxylic acid amide (82)

The title compound can be prepared by methods similar to those set forth by Anderson⁸ page 999.

### Example 24

Synthesis of 4-amino-8-(2′-C-methyl-β-D-ribofuranosyl)-7-oxo-7,8-dihydro-pyrido[2,3-d]pyrimidine-5-carboxylic acid amide (83)

The title compound can be prepared by methods similar to those set forth by Anderson⁸ page 1000.

### Example 25

Synthesis of 2,4-diamino-8-(2′-C-methyl-β-D-ribofuranosyl)-7-oxo-7,8-dihydro-pyrido[2,3-d]pyrimidine-5-carboxylic acid amide (84)

The title compound can be prepared by methods similar to those set forth by Anderson⁸ page 1000.

### Example 26

Synthesis of 8-(2′-C-methyl-β-D-ribofuranosyl)-2-methylsulfanyl-4,5-dioxo-3,4,5,8-tetrahydropyrido[2,3-d]pyrimidine-6-carboxylic acid amide (85)

Step 1. Synthesis of 2-Methylsulfanyl-4,5-dioxo-3,4,5,8-tetrahydro-pyrido[2,3-d]pyrimidine-6-carboxylic acid ethyl ester

4,5-dioxo-3,4,5,8-tetrahydro-pyrido[2,3-d]pyrimidine-6-carboxylic acid ethyl ester was synthesized as described in B. H.Rizkalla and A. D.Broom, J.Org.Chem. 1972, 37(25), 3980-3985.

Step 2. Synthesis of 8-(3,4-Bis-benzoyloxy-5-benzoyloxymethyl-3-methyl-tetrahydro-furan-2-yl)-2-methylsulfanyl-4,5-dioxo-3,4,5,8-tetrahydro-pyrido[2,3-d]pyrimidine-6-carboxylic acid ethyl ester

To a suspension of the product from Step 1 above (0.2 g, 0.71 mmol) in dry acetonitrile (3.5 mL), BSA (0.385 mL, 1.56 mmol) was added and the mixture refluxed under argon for 30 min. The resulting solution was cooled to room temperature and 1,2,3,5-tetra-O-benzoyl-2′-C-methyl β-D-ribofuranose (0.32 g, 0.55 mmol) in dry acetonitrile was added followed immediately by TMSOTf (0.513 mL, 2.84 mmol). The resulting reaction mixture was heated to reflux for 2 hours. The reaction was allowed to cool to room temperature then was concentrated in vacuo to an oily residue. The oily residue was taken up in EtOAc and washed 1× with saturated NaHCO₃ and the aqueous layer was re-extracted 2× with EtOAc. The organic fractions were combined, washed with H₂O, brine, and dried over Na₂SO₄ and concentrated in vacuo. The crude reaction was purified by column chromatography on silica gel using 10% methanol in methylene chloride for elution. The appropriate fractions were pooled, evaporated, and foamed from methylene chloride to get 0.406 g (100%) of the title compound.

Step 3. Synthesis of 8-(3,4-Dihydroxy-5-hydroxymethyl-3-methyl-tetrahydro-furan-2-yl)-2-methylsulfanyl-4,5-dioxo-3,4,5 8-tetrahydro-pyrido[2,3-d]pyrimidine-6-carboxylic acid amide.

The product from Step 2 above (0.2 g, 0.270 mmol) was dissolved in 40 mLs liquid ammonia and stirred at room temperature for 48 hours. The liquid ammonia was allowed to evaporate and the resulting yellow oily residue was purified by HPLC 0-20% Buffer B over 30 min at a flow rate of 10 mLs/min. Buffer A—0.1% triethylammonium acetate in water, Buffer B—0.1% triethylammonium acetate in CH₃CN. Pooled fractions containing nucleoside and evaporated in vacuo and dried by co-evaporation with absolute ethanol to yield 27 mg (25%) of the desired nucleoside.

MS: 397.13 (M−H).

H¹-NMR (DMSO-d6): 0.8 (s, 3H, 2′-CH₃), 2.5 (s, 3H, —CH3), 3.0-4.0 (m, 4H, sugar), 5.0-5.5 (m, 3H, —OH), 6.7 (s, 1H, 1′-H), 7.4 (s, 1H, —Ar), 8.8 and 9.2 (s, 2H, —NH₂).

### Example 27

Synthesis of 8-(2′-C-methyl-β-D-ribofuranosyl)-8H-pyrido[2,3-d]pyrimidine-2,4-dione (86)

The title compound can be prepared by methods similar to those set forth by Rizkalla⁹ on page 3979.

### Example 28

Synthesis of 1-(2′-C-methyl-β-D-ribofuranosyl)-1H-pyrido[2,3-d]pyrimidine-2,4-dione (87)

The title compound can be prepared by methods similar to those set forth by Rizkalla⁹ on page 3979.

### Example 29

Synthesis of 8-(2′-C-methyl-β-D-ribofuranosyl)-4-methylsulfanyl-5,6,7,8-tetrahydro-pyrido[2,3-d]pyrimidine (88)

The title compound can be prepared by methods similar to those set forth in Biorog. Khim., 1979, 5, 1369.

### Example 30

Synthesis of 3-(2′-C-methyl-β-D-ribofuranosyl)-6-methyl-3,7a-dihydro-1H-furo[2,3-d]pyrimidin-2-one (89)

The title compound can be prepared by methods similar to those set forth in De Clercq¹² page 666.

### Example 31

Synthesis of 3-(2′-C-methyl-β-D-ribofuranosyl)-3,5,6,7a-tetrahydro-1H-furo[2,3-d]pyrimidin-2-one (90)

The title compound can be prepared by making appropriate modifications to the methods set forth by Griengl¹⁴ on page 1680.

### Example 33

Synthesis of 7-(2′-C-methyl-β-D-ribofuranosyl)-4-methylsulfanyl-7H-pyrrolo[2,3-d]pyrimidine (92)

The title compound can be prepared by methods similar to those set forth by Seela¹⁷ page 1585.

### Example 34

Synthesis of 1-(2′-C-methyl-β-D-ribofuranosyl)-4-methylsulfanyl-1H-pyrrolo[2,3-d]pyrimidine (93)

The title compound can be prepared by methods similar to those set forth by Seela¹⁷ page 1585.

### Example 35

Synthesis of 3-(2′-C-methyl-β-D-ribofuranosyl)-3H-[1,2,4]triazolo[1,5-a]pyrimidin-7-one (94)

The title compound can be prepared by methods similar to those set forth in Winkley¹⁸ page 239.

### Example 36

Synthesis of 3-methyl-8-(2′-C-methyl-β-D-ribofuranosyl)-2-methylsulfanyl-3H,8H-pteridine-4,7-dione (95)

The title compound can be prepared by methods similar to those set forth by Hawkin³⁹, et al. page 2875.

### Example 37

Synthesis of 5-(2′-C-methyl-β-D-ribofuranosyl)pyridin-2-ylamine (96)

The title compound can be prepared by coupling the alternative the sugar f, prepared as described in Scheme 1, to the base prepared by methods similar to those described previously.²²⁻²³

### Example 38

Synthesis of 5-(2′-C-methyl-β-D-ribofuranosyl)-1H-pyridin-2-one (97)

The title compound can be prepared by coupling the alternative sugar f, prepared as described in Scheme 1, to the base prepared by methods similar to those described previously.²²⁻²³

### Example 39

Synthesis of 8-(2′-C-methyl-β-D-ribofuranosyl)-pyrazolo[1,5-a][1,3,5]triazin-4-ylamine(98)

The title compound can be prepared by coupling the alternative sugar f, prepared as described in Scheme 1, to the base prepared by methods similar to those described by Tam²⁵, et al. on page 1307. Other pyrazolotrazine C-nucleosides, for example compounds 99 and 100, may be prepared using this sugar (f) and other techniques well known in the art.²⁴⁻²⁷

### Example 41

Synthesis of 9-(2′-C-trifluoromethyl-β-D-ribofuranosyl)-N⁶-(2-aminoethyl)adenine (62)

The title compound can be prepared by methods similar to those set forth by Li³⁵, et al. and methods described herein. Trifluoromethylated ribofuranosyl derivates maybe coupled to a variety of bases, for example compounds 63, 64, 66 and 67, may be prepared by techniques described herein as well as methods well known in the art.

### Example 42

Synthesis of 1-(2′-C-ethenyl-β-D-ribofuranosyl)-1H-benzimidazole (73)

The title compound can be prepared by methods similar to those set forth by Sagi³⁸, et al. and methods described herein. Ethenylated ribofuranosyl derivates maybe coupled to a variety of bases, for example compounds 68-70, may be prepared by techniques described herein as well as methods well known in the art.

### Example 43

Synthesis of 1-(2′-C-ethynyl-β-D-ribofuranosyl)-1H-benzimidazole (79)

The title compound can be prepared by methods similar to those set forth by Sagi³⁸, et al. and methods described herein. Ethynylated ribofuranosyl derivates maybe coupled to a variety of bases, for example compounds 74-76, may be prepared by techniques described herein as well as methods well known in the art.

### Example 44

Synthesis of 1-(2′-C-methyl-β-D-ribofuranosyl)-4-nitroindole (104)

The title compound can be prepared by methods similar to those set forth in Yokoyama⁴³, et al. Other Indole nucleosides can be prepared by coupling ribofuranosyl derivatives to a variety of indole, for example compounds 105, maybe prepared by techniques described herein as well as methods well known in the art.⁴³

### Example 45

Synthesis of 9-(2′-C-methyl-β-D-ribofuranosyl)-6-(azetidin-1-yl)purine (107).

Compound 107 was synthesized from azetidine and nucleoside 51 as described in Example 9, step 4.

MS 323.32 (M+H)

H¹-NMR (DMSO-d6): 0.76 (s, 3H, 2′-CH₃), 3.25-3.45 (m, 4H, methylene), 3.10-4.10 (m, sugar and azetidine), 5.98 (s, 1H, 1′-H), 8.35 and 8.68 (s, 1H, purine).

### Example 46

Synthesis of 9-(2′-C-methyl-β-D-ribofuranosyl)-6-(pyrrolidin-1-yl)purine (108).

Compound 108 was synthesized from pyrrolidine and nucleoside 51 as described in Example 9, step 4.

MS 336.32 (M+H)

H¹-NMR (DMSO-d6): 0.77 (s, 3H, 2′-CH₃), 2.00 (m, 4H, pyrrolidine), 3.43-4.14 (m, sugar and pyrrolidine), 5.98 (s, 1H, 1′-H), 8.36 and 8.72 (s, 1H, purine).

### Example 47

Synthesis of 9-(2′-C-methyl-β-D-ribofuranosyl)-6-(piperidin-1-yl)purine (57).

Compound 57 was synthesized from pyrrolidine and nucleoside 51 as described in Example 9, step 4.

MS 350.37 (M+H)

H¹-NMR (DMSO-d6): 0.78 (s, 3H, 2′-CH₃), 1.62 (m, 6H, piperidine), 3.43-3.88 (m, sugar and piperidine), 4.01-4.02 (d, 1H, 3′-H) 5.97 (s, 1H, 1′-H), 8.28 and 8.58 (s, 1H, purine).

### Example 48

Synthesis of 9-(2′-C-methyl-β-D-ribofuranosyl)-6-(hydroxylamino)purine (109) and 9-(2′-C-methyl-β-D-ribofuranosyl)-hypoxanthine (110).

Sulfonyl 51 (0.2 mmol) was dissolved in 3 mL of dry ethanol, solution of hydroxylamine (prepared as described by P. K.Chang, J.Med.Chem., 1965, 8, 884) was added (2 mM) and the mixture was refluxed for 1 h and than concentrated in vavuo. The residue was dissolved in DMF (5 mL) and purified by HPLC 20-100% B in 30 min, flow 10 mL/min. A-0.2% triethylammonium acetate in water, B-0.2% triethylammonium acetate in CH₃CN.

The fractions contained the mixture of protected nucleosides 109 and 110 were evaporated, dissolved in MeOH, treated with HCl/MeOH for 5 min at 0° C. and the mixture of nucleosides 109 and 110 (3:1) was precipitated with ether. The mixture was separated by HPLC, 0-20% B in 30 min, buffers as described above.

Corresponding fractions were combined, evaporated, co-evaporated with water (3×10 mL), dissolved in methanol (1 mL) and precipitated with ether (35 mL) to yield white solid.

9-(2′-C-methyl-β-D-ribofuranosyl)-N⁶-(hydroxylamino)purine (109)

MS: 283.19 (M+H),

δₘₐₓ 261.5 nm,)

H¹-NMR (DMSO-d6): 0.68 (s, 3H, 2′-CH₃), 3.81-4.04 (m, 2H, 5′-H) 4.07 (t, 1H, 4′-H), 4.17-4.20 (d, 3′-H), 6.06 (s, 1H, 1′-H), 8.06 and 8.53 (s, 1H, purine).

9-(2′-C-methyl-β-D-ribofuranosyl)-hypoxanthine (110).

MS: 298.38 (M+H),

δₘₐₓ 249.5 nm,

H¹-NMR (D)MSO-d6): 1.09 (s, 3H, 2′-CH₃), 3.85-4.24 (m, 3H, sugar), 6.16 (s, 1H, 1′-H), 8.21 and 8.62 (s, 1H, hypoxanthine).

### Example 49

Synthesis of 9-(2′-C-methyl-β-D-ribofuranosyl)-6-methoxyaminopurine (111).

Compound 111 was synthesized from methoxylamine and nucleoside 51 as described in Example 9, step 4.

MS 312.41 (M+H);

H¹-NMR (DMSO-d6): 0.91 (s, 3H, 2′-CH₃), 3.82-4.04 (m, 7H, sugar), 3.95 (s, O—CH₃), 6.01 (s, 1H, 1′-H), 8.22 and 8.88 (s, 1H, adenine).

### Example 50

Synthesis of 9-(2′-C-methyl-β-D-ribofuranosyl)-6-hydrazinopurine (55).

Nucleoside 55 was synthesized from sulnonyl derivative 51 and hydrazine as described in Example 9, step 4.

MS 297.31 (M+H)

H¹-NMR (DMSO-d6): 0.80 (s, 3H, 2′-CH₃), 3.80-4.00 (m, 7H, sugar), 6.02 (s, 1H, 1′-H), 8.47 and 8.77 (s, 1H, purine).

### Example 51

Synthesis of 9-(2′-C-methyl-β-D-ribofuranosyl)-6—N-methylhydrazinopurine (112).

Nucleoside 112 was synthesized from sulnonyl derivative 51 and hydrazine as described in Example 9, step 4.

MS 313.72 (M+H)

H¹-NMR (DMSO-d6): 0.68 (s, 3H, 2′-C₃), 3.80-4.00 (m, 7H, sugar), 3.88 (s, N—CH₃), 5.90 (s, 1H, 1′-H), 7.68 and 8.21 (s, 1H, purine).

### Example 52

9-(2′-C-methyl-β-D-ribofuranosyl)-6-(3.6-dihydro-2H-pyridin-1-yl)purine (113).

Compound 113 was synthesized from 3,6-dihydropyridine and nucleoside 51 as described in Example 9, step 4.

MS 348.32 (M+H)

H¹-NMR (DMSO-d6): 0.88 (s, 3H, 2′-CH₃), 3.10-3.40 (m, 6H, CH2-tetrahydropyridine), 3.80-4.00 (m, 7H, sugar), 5.80-5.98 (m, 2H, CH-tetrahydropyridine), 6.01 (s, 1H, 1′-H), 8.23 and 8.48 (s, 1H, purine).

### Example 53

Synthesis of 9-(2′-C-methyl-β-D-ribofuranosyl)-6-(3,4-dihydro-1H-isoquinolin-2-yl)purine (114).

Compound 114 was synthesized from 3,4-dihydroisoquinoline and nucleoside 51 as described in Example 9, step 4.

MS 398.53 (M+H)

H¹-NMR (DMSO-d6): 0.88 (s, 3H, 2′-CH₃), 2.25-2.31 and 2.90-3.00 (m, 2H, methylene), 3.10-3.40 (m, 6H, CH₂-tetrahydropyridine), 3.80-4.00 (m, 4H, sugar), 5.20-5.35 (m, 3H, OH-sugar), 6.01 (s, 1H, 1′-H), 7.16-7.25 (m, 4H, benzene), 8.27 and 8.53 (s, 1H, purine).

### Example 54

Preparation of 9-(2′-C-methyl-β-D-ribofuranosyl)-6-(1,3,4,9-tetrahydro-beta-carbolin-2-yl) purine (33).

Compound 33 was synthesized from 3,4-dihydroisoquinoline and nucleoside 51 as described in Example 9, step 4.

MS 437.43 (M+H)

H¹-NMR (DMSO-d6): 0.89 (s, 3H, 2′-CH₃), 2.98 (m, 2H, methylene), 3.40-4.00 (m, sugar and methylene of tetrahydopyridine), 4.05 (d, 3′-H), 6.05 (s, 1H, 1′-H), 6.90-7.05 (m, 2H, aromatic), 7.29-7.40 (m, 2H, aromatic), 8.32 and 8.65 (s, 1H, purine), 10.99 (s, 1H, NH).

### Example 55

Synthesis of 7-(2′-C-methyl-β-D-ribofuranosyl)-4-hydroxylamino-pyrrolo[2,3-d]pyrimidine (117)

Step 1. Synthesis of 7-(2′-C-methyl-β-D-ribofaranosyl)-4-chloro-pyrrolo[2,3-d]pyrimidine (141) was prepared as described in WO 02/057287, p 27-30.

Step 2. 7-(2′-C-methyl-β-D-ribofuranosyl)-4-hydroxylamino-pyrrolo[2,3-d]pyrimidine (117).

Nucleoside 141 (300 mg, 1 mmol) was dissolved in dry ethanol (10 mL), solution of hydroxylamine (prepared as described by P. K.Chang, J.Med.Chem., 1965, 8, 884) was added (10 mM) and the mixture was refluxed for 1 h and than concentrated in vavuo. The residue was purified by HPLC 0-30% B in 30 min, flow 10 mL/min. A—0.2% triethylammonium acetate in water, B—0.2% triethylammonium acetate in CH₃CN. Corresponding fractions were combined, evaporated, co-evaporated with water (3×10 mL), dissolved in methanol (1 mL) and precipitated with ether (35 mL) to yield 117 as white solid.

### Example 56

Synthesis of 7-(2′-C-methyl-β-D-ribofuranosyl)-4-methoxylamino-pyrrolo [2,3-d]pyrimidine (118)

Nucleoside 118 was prepared from the nucleoside 141 (example 55, step 1) substituting methoxylamine for hydroxylamine.

### Example 57

Synthesis of 1-(2′-C-methyl-β-D-ribofuranosyl)-4-hydroxylamino-pyrazolo[3,4-d]pyrimidine (120)

Step 1. Synthesis of 2.3,5-tri-O-benzoyl-2′-methyl-1,5-dihydro-pyrazolo[3,4-d]pyrimidin-4-one (142).

Nucleoside 142 was synthesized as described in example I by substitution of 6-bromopurine for 1,5-dihydro-pyrazolo[3,4-d]pyrimidin-4-one

Step 2. Synthesis of 2,3,5-tri-O-benzoyl-2′-methyl-4-chloro-pyrazolo[3,4-d]pyrimidine (143)

Nucleoside 142 was dissolved in toluene, 10 equivalents of SOCl₂ was added and the mixture was heated at 50° C. for 2 hours. The solvents were evaporated in vacuum, the residue was co-evaporated with toluene and purified by flash chromatography on silica gel (toluene-ethyl acetate, 9:1 v/v). Corresponding fractions were evaporated, dissolved in 10 mL of methanol and 5 mL NH₄OH was added. Reaction mixture was kept at room temperature overnight and evaporated. The titled nucleoside was isolated by HPLC as described in example 55, step2.

Step 3. 1-(2′-C-methyl-β-D-ribofuranosyl)-4-hydroxylamino-pyrazolo[3,4-d]pyrimidine (120)

Nucleoside 143 was transformed to nucleoside 120 as it is described in example 55, step 2.

### Example 58

Synthesis of 1-(2′-C-methyl-β-D-ribofuranosyl)-4-methoxylamino-pyrazolo [3,4-d]pyrimidine (119)

Nucleoside 119 was prepared from the nucleoside 143 (example 57, step 3) substituting hydroxylamine for methoxylamine.

### Example 59

Synthesis of 7-(2′-C-methyl-β-D-ribofuranosyl)-5-chloro-4-hydroxylamino pyrrolo[2,3-d]pyrimidine (123)

Nucleoside 117 (0.1 mmol) is dissolved in DMF (0.5 mL) and cooled to 0° C. N-chlorosuccinimide (NCS) (0.1 mmol) dissolved in DMF (0.5 mL) is then added dropwise and the reaction stirred for 30 min at 0° C. and 30 min at room temperature. The reaction is quenched with methanol (5 mL) and then concentrated. Column chromatography (SiO₂) with MeOH/DCM affords 123.

### Example 60

Synthesis of 7-(2′-C-methyl-β-D-ribofuranosyl)-5-bromo-4-hydroxylamino pyrrolo[2,3-d]pyrimidine (124)

Nucleoside 124 is prepared in the same manner as for 123, substituting N-bromosuccinimide (NBS) for NCS.

### Example 61

Synthesis of 7-(2′-C-methyl-β-D-ribofuranosyl)-5-methyl-4-hydroxylamino-pyrrolo[2,3-d]pyrimidine (125)

Step 1: Nucleoside 141 (1 mmol) is dissolved in DMF (5 mL) and cooled to 0° C. NBS (1 mmol) dissolved in DMF (5 mL) is then added dropwise and the reaction stirred for 30 min at 0° C. and 30 min at room temperature. The reaction is quenched with methanol (50 mL) and then concentrated. Column chromatography (SiO₂) with MeOH/DCM affording the 7-bromo-6-chloro-7-deazapurine riboside.

Step 2: The nucleoside from Step 1 (0.5 mmol) is dissolved in 10% aqueous dioxane (2.5 mL) and potassium carbonate (1.5 mmol) and palladium tetrakis(triphenylphosphine) are added followed by trimethylboroxine (0.5 mmol). The reaction is refluxed for 18 hrs. then filtered through Celite and concentrated. Column chromatography (SiO₂) with MeOH/DCM affording the 7-methyl-6-chloro-7-deazapurine riboside.

Step 3: Nucleoside 125 is synthesized as described in Example 55, step 2 using hydroxylamine.

### Example 62

Synthesis of 7-(2′-C-methyl-β-D-ribofuranosyl)-5-ethyl-4-hydroxylamino-pyrrolo[2,3-d]pyrimidine (128)

Step 1: The nucleoside from Example 61, Step 1 (0.1 mmol) is dissolved in THF (1 mL) and then palladium tetrakis(triphenylphosphine) is added. To this reaction is then added diethyl zinc and the reaction heated to reflux for 6 hours. The reaction is quenched with aqueous NH₄Cl and extractively worked up. Column chromatography (SiO₂) with MeOH/DCM affording the 7-ethyl-6-chloro-7-deazapurine riboside.

Step 2:

Nucleoside 128 is synthesized as described in Example 55, step 2 using hydroxylamine.

### Example 63

Synthesis of 7-(2′-C-methyl-β-D-ribofuranosyl)-5-cyano-4-hydroxylamino-pyrrolo[2,3-d]pyrimidine (126)

Step 1: To the nucleoside from Example 61, step 1 (0.5 mmol) ) is dissolved in THF (5 mL) and then palladium tetrakis(triphenylphosphine) is added. To this reaction is then added zinc cyanide and the reaction heated to reflux for 6 hours. The reaction is quenched with aqueous NH₄Cl and extractively worked up. Column chromatography (SiO₂) with MeOH/DCM affording the 7-cyano-6-chloro-7-deazapurine riboside.

Step 2:

Nucleoside 126 is synthesized as described in Example 55, step 2 using hydroxylamine.

### Example 64

Synthesis of 7-(2′-C-methyl-β-D-ribofuranosyl)-4-hydroxylamino-pyrrolo [2,3-d]pyrimidine 5-carboxyl amide (127)

Step 1: The nucleoside from Example 63, step 1 (0.5 mmol) is dissolved in anhydrous ethanol (10 mL) and then saturated with anhydrous HCl. The reaction is stirred at room temperature overnight and then concentrated. The residue is redissolved in ethanol (5 mL) and then water (1 mL) is added and the reaction stirred for 2 hours. The solution is concentrated and purified by column chromatography (SiO₂) with MeOH/DCM affording the 7-carboxamide-6-chloro-7-deazapurine riboside.

Step 2: Nucleoside 127 is synthesized as described in Example 55, step 2 using hydroxylamine.

### Example 65

Synthesis of 7-(2′-C-methyl-β-D-ribofuranosyl)-5-bromo-4-methoxylamino-pyrrolo[2,3-d]pyrimidine (129)

Nucleoside 129 is synthesized from 118 as described in Example 60.

### Example 66

Synthesis of 7-(2′-C-methyl-β-D-ribofuranosyl)-5-methyl-4-methoxylamino-pyrrolo[2,3-d]pyrimidine (130)

Nucleoside 130 is synthesized as described in Example 55, step 2, substituting methoxylamine for hydroxylamine.

### Example 67

Synthesis of 7-(2′-C-methyl-β-D-ribofuranosyl)-5-cyano-4-methoxylamino-pyrrolo[2,3-d]pyrimidine (131)

The nucleoside from example 61, step 2 is converted to 131 as described in Example 66.

### Example 69

Synthesis of 7-(2′-C-methyl-β-D-ribofuranosyl)-4-methoxylamino-pyrrolo [2,3-d]pyrimidine 5-carboxyl amide (132)

The nucleoside from example 63, step 1 is converted to 132 as described in Example 66.

### Example 70

Synthesis of 1-(2′-C-methyl-β-D-ribofuranosyl)-3-bromo-4-hydroxylamino-pyrazolo[3,4-d]pyrimidine (133)

Nucleoside 120 is converted to 133 as described in Example 60.

### Example 71

Synthesis of 1-(2′-C-methyl-β-D-ribofaranosyl)-3-methyl-4-hydroxylamino-pyrazolo[3,4-d]pyrimidine (134)

Nucleoside 134 is synthesized from 143 using conditions described in Example 61.

### Example 72

Synthesis of 1-(2′-C-methyl-β-D-ribofuranosyl)-3-cyano-4-hydroxylamino-pyrazolo[3,4-d]pyrimidine (135)

Nucleoside 135 is synthesized from 143 using conditions described in Example 63.

### Example 73

Synthesis of 1-(2′-C-methyl-β-D-ribofuranosyl) -4-hydroxylamino-pyrazolo [3,4-d]pyrimidine-3-carboxamide (136)

Nucleoside 136 is synthesized from 143 using conditions described in Example 64.

### Example 74

Synthesis of 1-(2′-C-methyl-β-D-ribofuranosyl)-3-bromo-4-methoxylamino-pyrazolo[3,4-d]pyrimidine (137)

Nucleoside 137 is synthesized from 119 using conditions described in Example 61.

### Example 75

Synthesis of 1-(2′-C-methyl-β-D-ribofuranosyl)-3-methyl-4-methoxylamino-pyrazolo[3,4-d]pyrimidine (138)

Nucleoside 138 is synthesized from 143 using conditions described in Example 61, substituting methoxylamine for hydroxylamine.

### Example 76

Synthesis of 1-(2′-C-methyl-β-D-ribofuranosyl)-3-cyano-4-methoxylamino-pyrazolo[3,4-d]pyrimidine (139)

Nucleoside 139 is synthesized from 143 using conditions described in Example 63, substituting methoxylamine for hydroxylamine.

### Example 77

Synthesis of 1-(2′-C-methyl-β-D-ribofuranosyl) -4-methoxylamino-pyrazolo [3,4-d]pyrimidine-3-carboxamide (140)

Nucleoside 140 is synthesized from 143 using conditions described in Example 64, substituting methoxylamine for hydroxylamine.

### Example 78

Synthesis of 2′-C-methyl-β-D-ribofuranosyl-6-methylthio-purine (150)

Step 1. Synthesis of 2′,3′,5′-Tri-O-benzoyl-2′-C-methyl-β-D-ribofuranosyl-6-methylthio-purine.

6-Methylthio-purine (1.43 g, 8.6 mmolol)) was suspended in 100 mL of dry CH₃CN, bis-trimethylsilylacetamide (BSA) was added (5 mL, 20 mmolol) and the mixture was refluxed until the clear solution was formed (about 30 min). 1,2,3,5-Tetra-O-benzoyl-2′-C-methyl β-D-ribofuranose (4 g, 6.9 mmolol) was added followed by trimethylsilyl trifluoromethane sulfonate (TMSOTf) (5 mL). The mixture was refluxed for 4 hours, disappearance of the sugar was controlled by TLC in hexane-ethyl acetate (l:1 v/v). Solution of 10% NaHCO₃ was added and the benzoylated nucleoside was extracted with ethyl acetate. Water fraction was extracted with organic (2×30 mL). Combined organic fractions were washed with water, dried over Na₂SO₄ and evaporated. The titled nucleoside was isolated by column chromatography on silica gel using 5% ethyl acetate in toluene as eluent with 74% yield.

MS: 625.72 (M+H);

H¹-NMR (CDCl₃): 1.59 (s, 3H, 2′-CH₃), 2.74 (s, 3H, SCH₃), 4.70-4.80 & 5.90-5.00 (m, 3H, H-4′ and H-5′a,b), 6.23 (d, 1H, H-3′), 6.80 (s, 1H, H-1′), 7.25-8.20 (m, 15H, benzoyl), 8.20 & 8.80 (s, 2H, purine).

Step 2. Synthesis of 2′-C-methyl-β-D-ribofuranosyl-6-methylthio-purine.

The compound isolated in step 1 was dissolved in methanol saturated with K₂CO₃. After 20 min, the solvent was evaporated and the title compound was purified by flash chromatograpy in 10% methanol in chloroform.

MS: 313.38 (M+H);

H¹-NMR (DMSO-d6): 0.89 (s, 3H, 2′-CH₃), 2.82 (s, 3H, SCH₃), 3.62-4.15 (m, 4H, sugar), 5.23-5.31 (m, 2H, sugar), 5.40 (s, 1H, H-3′), 6.01 (s, 1H, H-1′), 8.20 & 8.80 (s, 2H, purine).

### Example 79

Synthesis of 2′-C-methyl-β-D-ribofuranosyl-6-phenyladenine (155)

6-Phenyl-adenine (315 mg, 1.5 mmol) was suspended in 20 mL of dry CH₃CN, BSA was added (0.4 mL) and the mixture was refluxed until the clear solution was formed (about 30 min). 1,2,3,5-Tetra-O-benzoyl-2′-C-methyl β-D-ribofuranose was added followed by trimethylsilyl trifluoromethane sulfonate (0.2 mL). The mixture was refluxed for 4 hours, disappearance of the sugar was controlled by TLC in hexane-ethyl acetate (1:1 v/v). Solution of 10% NaHCO₃ was added and the benzoylated nucleoside was extracted with ethyl acetate. Water fraction was extracted with organic (2×30 mL). Combined organic fractions were washed with water, dried over Na₂SO₄ and evaporated. The residue was dissolved in 20 mL of NH₃/methanol and left overnight at ambient temperature. The reaction mixture was concentrated and purified by column chromatography on silica gel using ethyl acetate/iso-propanol/water (9:1:2, upper phase) as eluent. The title nucleoside was dissolved in methanol and precipitated with ether with 75% yield.

MS: 358.51 (M+H);

H¹-NMR (DMSO-d6): 0.81 (s, 3H, 2′-CH₃), 2.82 (s, 3H, SCH₃), 3.80-4.20 (m, 4H, H-4′, H-5′a,b, HO-5′), 5.20-5.41 (m, 3H, H-3′, HO-2′, HO-3′), 6.01 (s, 1H, H-1′), 6.90-7.10 (t, 1H, 4-phenyl), 7.28-7.32 (t, 2H, 3,5-phenyl), 7.90 (d, 2H, 2,6-phenyl), 8.40 & 8.62 (s, 2H, purine), 9.90 (s, 1H, NH).

### Example 80

Synthesis of 2′-C-methyl-β-D-ribofuranosyl-6-(2-dimethylamino-ethylamino)purine

Step 1. Synthesis of 9-(5′-O-monomethoxytriphenylmethyl-2′-C-methyl-β-D-ribofuranosyl)-6-(methylsulfanyl).

Compound 150(1.5 g, 5 mmol) was dissolved in 30 mL of dry pyridine, p-anisylchlorodiphenylmethane (7.5 mmol) was added and reaction was kept at room temperature for 2 days. The solvent was evaporated and the residue was distributed between ethyl acetate and water. The organic phase was washed with 10% aqueous NaHCO₃, water, dried with NaSO₄ and evaporated. The crude oil was purified by column chromatography on silica gel using 5% methanol in chloroform. The fractions containing the title nucleoside were combined, evaporated and freeze-dried from benzene to yield 2.1 g (74%) of nucleoside the desired product as a white solid foam.

MS: 585.96 (M+H),

H¹-NMR (CDCl₃): 0.99 (s, 3H, 2′-CH₃), 2.76 (s, 3H, SCH₃), 3.80 (s, 3H, CH₃-trityl)3.50-3.55, 4.10-4.18 & 4.20-4.30 (m, 4H, sugar), 5.30 (d, 1H, H-3′), 6.08 (s, 1H, H-1′), 7.20-7.50 (m, 14H, trityl), 8.20 & 8.68 (s, 2H, purine).

Step 2. Synthesis of 9-(5′-O-monomethoxytriphenylmethyl-2′-C-methyl-β-D-ribofuranosyl)-6-(methylsulfonyl)purine

The nucleoside prepared in Step 1 above (2 g, 3.4 mmol) was dissolved in 5 mL of dry acetonitrile, 8.2 mL of 1M solution of 3-chloroperoxybezoic acid was added and reaction mixture was kept at room temperature for 1 hour. The reaction mixture was distributed between water and chloroform. The organic fraction was washed with 10% aqueous NaHCO₃, water, dried and evaporated to yield the titled compound in 95% yield.

MS: 617.83 (M+H).

Step 3. Synthesis of 9-(2′-C-methyl-β-D-ribofuranosyl)-6-(2-dimethylamino-ethylamino)purine

9-(5′-O-monomethoxytriphenylmethyl-2′-C-methyl-β-D-ribofuranosyl)-6-(methylsulfonyl)purine (0.2 mmol) was dissolved in 3 mL of dry acetonitrile and 2-dimethylamino-ethylamine was added (2 mmol). The mixture was refluxed for 1 h and then concentrated in vacuo. The residue was dissolved in DMF (5 mL) and purified by HPLC 20-100% B in 30 min, flow 10 mL/min. A—0.2% triethyl-ammonium acetate in water, B—0.2% triethylammonium acetate in CH₃CN. The fractions contained the protected 9-(2′-C-methyl-β-D-ribofuranosyl)-6-(2-dimethylamino-ethylamino)purine were evaporated, dissolved in MeOH, treated with HCl/MeOH for 5 min at 0° C. and the title compound was precipitated with ether. The title product was separated by HPLC, 0-20% B in 30 min (buffers described above). Corresponding fractions were combined, evaporated, co-evaporated with water (3×10 mL), dissolved in methanol (1 mL) and precipitated with ether (35 mL) to yield the title compound as a white solid.(yield: 55% based on 9-(5′-O-monomethoxytriphenylmethyl-2′-C-methyl-β-D-ribofuranosyl)-6-(methylsulfonyl)purine)

MS 338.92 (M+H)

H¹-NMR (DMSO-d6): 0.78 (s, 3H, 2′-CH₃), 1.62 (m, 6H, piperidine), 2.76-2.88 (s, 9H, methyl-N), 3.25-3.45 (m, 4H, methylene), 3.53-4.10 (m, 7H, sugar), 5.98 (s, 1H, 1′-H), 8.35 and 8.65 (s, 1H, purine).

### Example 81

Synthesis of 9-(2′-C-methyl-β-D-ribofuranosyl)benzimidazole (60) GL048795

The title compound was prepared as described above in Example 79 using benzimidazole as heterocyclic base.

MS 267.32. (M+H)

H¹-NMR (DMSO-d6): 0.81 (s, 3H, 2′-CH₃), 3.68-4.20 (m, 4H, sugar), 5.25-5.30 (m, 2H, sugar), 5.40 (s, 1H, H-3′), 6.10 (s, 1H, H-1′), 8.87, 9.00 & 9.10 (3s, purine).

### Example 82

Synthesis of 9-(2′-C-methyl-β-D-ribofuranosyl)-6-(2-(1H-imidazol-4-yl)-ethylamino)purine (156)

Compound 156 was synthesized from 2-(2H-imidazole-4-yl)-ethylamine and 9-(5′-O-monomethoxytriphenylmethyl-2′-C-methyl-β-D-ribofuranosyl)-6-(methylsulfonyl)purine as described in Example 80, step 3.

MS 376.78 (M+H)

H¹-NMR (DMSO-d6): 0.80 (s, 3H, 2′-CH₃), 3.25-3.45 (m, 4H, methylene), 3.53-4.05 (m, 7H, sugar), 5.99 (s, 1H, 1′-H), 7.48 and 9.09 (s, 1H, purine), 8.35 and 8.65 (bs, 0.7H, imidazole)

### Example 83

Synthesis of 9-(2′-C-methyl-β-D-ribofuranosyl)-6-(2-piperidin-1-yl-ethylamino)purine (157)

The title compound was synthesized from 2-piperidin-1-yl-ethylamine and 9-(5′-O-monomethoxytriphenylmethyl-2′-C-methyl-β-D-ribofuranosyl)-6-(methylsulfonyl)purine as described in Example 80, step 3.

MS 293.58 (M+H);

H¹-NMR (DMSO-d6): 0.88 (s, 3H, 2′-CH₃), 1.40 (bs, 2H, methylene), 1.65-1.82 (m, 4H, 3.25-3.45 (m, 4H, methylene), 3.10-4.15 (m, 10H, sugar & piperidine), 5.99 (s, 1H, 1′-H), 8.35 (s, 1H, purine), 8.60 (bs, 1.5H, purine & NH).

### Example 84

Synthesis of 9-(2′-C-methyl-β-D-ribofuranosyl)-6-(cyclopropylamino)purine (158)

The title compound was synthesized from cyclopropylamine and 9-(5′-O-monomethoxytriphenylmethyl-2′-C-methyl-β-D-ribofuranosyl)-6-(methylsulfonyl) purine as described in Example 80, step 3.

MS 322.43 (M+H);

H¹-NMR (DMSO-d6): 0.88 (s, 3H, 2′-CH₃), 0.21-0.32 (m, 5H, cyclopropane), 3.53-4.05 (m, 7H, sugar), 5.99 (s, 1H, 1′-H), 8.68 and 8.99 (s, 1H, purine),

### Example 85

Synthesis of 9-(2′-C-methyl-β-D-ribofuranosyl)-6-(cyclopentylamino)purine (159)

The title compound was synthesized from cyclopentylamine and 9-(5′-O-monomethoxytriphenylmethyl-2′-C-methyl-β-D-ribofuranosyl)-6-(methylsulfonyl) purine as described in Example 80, step 3.

MS 350.64 (M+H);

H¹-NMR (DMSO-d6): 0.88 (s, 3H, 2′-CH₃), 1.47-1.65 (m, 9H, cyclopentane), 3.86-4.86 (m, 7H, sugar), 6.10 (s, 1H, 1′-H), 8.47 and 8.79 (s, 1H, purine), 11.5 (s, 1H, NH).

### Example 86

Synthesis of 9-(2′-C-methyl-β-D-ribofuranosyl)-6-(cyclohexylamino)purine (160)

The title compound was synthesized from cyclohexylamine and 9-(5′-O-monomethoxytriphenylmethyl-2′-C-methyl-β-D-ribofuranosyl)-6-(methylsulfonyl) purine as described in Example 80, step 3.

MS 364.64 (M+H);

H¹-NMR (DMSO-d6): 0.86 (s, 3H, 2′-CH₃), 1.30-1.42 (m, 10H, methylene), 2.58-2.62 (m, 1H, methine), 3.86-4.86 (m, 7H, sugar), 6.10 (s, 1H, 1′-H), 8.24 and 8.98 (s, 1H, purine), 11.5 (s, 1H, NH).

### Example 87

Synthesis of 9-(2′-C-methyl-β-D-ribofuranosyl)-6-(6-Fluoro-1,3,4,9-tetrahydro-β-carbolin-2-yl)purine (163)

The title compound was synthesized from 6-fluoro-2,3,4,9-tetrahydro-1H-beta-carboline and 9-(5′-O-monomethoxytriphenylmethyl-2′-C-methyl-β-D-ribofuranosyl)-6-(methylsulfonyl)purine as described in Example 80, step 3.

MS 455.69 (M+H);

H¹-NMR (DMSO-d6): 0.82 (s, 3H, 2′-CH₃), 1.10-1.40 (m, 6H, methylene), 3.00-4.00 (m, 6H, sugar), 4.18-4.21 (d, 1H, H-3′), 6.05 (s, 1H, H-1′), 6.90-6.95 (m, 1H, indole), 7.30-7.35 (m, 2H, indole), 8.36 & 8.67 (s, 1H, purine), 11.5 (s, 1H, NH).

### Example 88

Synthesis of 9-(2′-C-methyl-β-D-ribofuranosyl)-6-(3,6-dihydro-2H-pyridin-1-yl)purine (164)

The title compound was synthesized from 1,2,3,6-tetrahydro-pyridine and 9-(5′-O-monomethoxytriphenylmethyl-2′-C-methyl-β-D-ribofuranosyl)-6-(methylsulfonyl)purine as described in Example 80, step 3.

MS 348.49 (M+H);

H¹-NMR (DMSO-d6): 0.90 (s, 3H, 2′-CH₃), 1.50-1.63 (m, 2H, methine), 2.10-3.20 (m, 6H, tetrahydropyridine), 3.80-4.10 (m. 3H, sugar), 5.20-5.40 (m, 3H, sugar), 6.00 (s, 1H, H-1′), 8.22 & 8.55 (s, 1H, purine).

### Example 89

Synthesis of 1-(2′-C-methyl-β-D-ribofuranosyl)-5-aminobenzimidazole and 1-(2′-C-methyl-β-D-ribofuranosyl)-6-aminobenzimidazole GL048950

Step 1. Synthesis of 1-(2′-C-methyl-β-D-ribofuranosyl)-5-nitrobenzimidazole and 1-(2′-C-methyl-β-D-ribofuranosyl)-6-nitrobenzimidazole

The mixture of nitronucleosides was prepared with the yield 82% as described above in Example 79 using 5-nitrobenzimidazole as heterocyclic base.

MS: 310.34 (M+H);

H¹-NMR (DMSO-d6): 0.71 & 0.72 (s, 3H, 2′-CH₃), 3.23-4.00 (m, 4H, sugar), 5.19-5.33 (m, 1H, sugar), 5.41 & 5.50 (2s, 1H, H-3′), 6.05 & 6.13 (2s, 1H, H-1′), 7.80-9.00 (4H, benzimidazole).

Step 2. Synthesis of 1-(2′-C-methyl-β-D-ribofuranosyl)-5-aminobenzimidazole and 1-(2′-C-methyl-β-D-ribofuranosyl)-6-aminobenzimidazole

The mixture of nitro nucleosides prepared in Step 1 above was dissolved in methanol and hydrogenated over 10% Pd/C at 25psi for 40 min. Catalyst was filtered and thoroughly washed with methanol, solution was concentrated and the residue purified by column chromatography as described in Example 79 to yield inseparable mixture of 5- and 6-aminobenzimidazole nucleosides.

MS 280.32 (M+H)

H¹—NMR (DMSO-d6): 0.84 & 0.87 (s, 3H, 2′-CH₃), 3.23-4.00 (m, 8H, sugar), 5.19-5.33 (m, 4H, sugar), 4.76 & 4.99 (2s, 1H, H-3′), 5.68 & 5.75 (2s, 1H, H-1′), 6.49-7.29 (4H, benzimidazole), 8.21 & 8.29 (2s, 1H, NH₂).

### Example 91

Preparation of 9-(2′-C-methyl-β-D-ribofuranosyl)-6-(tetramethyl-guanidino)purine (178)

The title compound was synthesized from tetramethylguanidine and 9-(5′-O-monomethoxytriphenylmethyl-2′-C-methyl-β-D-ribofuranosyl)-6-(methylsulfonyl) purine as described in Example 80, step 3.

MS 380.49 (M+H);

H¹-NMR (DMSO-d6): 0.90 (s, 3H, 2′-CH₃), 2.90 (s, 12H, CH₃), 3.20-4.15 (m. 7H, sugar), 6.00 (s, 1H, H-1′), 8,48 & 8.85 (s, 1H, purine).

### Example 92

Synthesis of 2′-C-methyl-β-D-ribofuranosyl-purine-6-carboxamide (208)

Step 1. Synthesis of 1′,2′,3′,5′-tetra-O-benzoyl-2′-C-methyl-6-carbonitrile-purine

9-(5′-O-monomethoxytriphenylmethyl-2′-C-methyl-β-D-ribofuranosyl)-6-(methylsulfanyl)purine (example 80, step 1) (624 mg, 1 mmol) was dissolved in 5 mL of dry acetonitrile, 3 mL of a 1 M solution of 3-chloroperoxybenzoic acid was added and reaction mixture was kept at room temperature for 1 hour. The reaction mixture was distributed between water and chloroform. The organic fraction was washed with 10% aqueous NaHCO₃, water, dried and evaporated to yield 6-mesyl-nucleoside with 95% yield.

MS: 657.83 (M+H).

The product was dissolved in DMF and NaCN (2 equiv.) was added. The reaction mixture was stirred at room temperature for 2.5 h to provide a yellow solution. The solvent was evaporated in vacuo to leave a residue, which was partitioned with chloroform and water. Organic portion was washed with water, 10% NaHCO₃ and water again. The chloroform portion was dried and evaporated. The compound was isolated by column chromatography on silica gel using 5% of methanol in chloroform for elution. The corresponding fractions were evaporated to yield the desired product (50%) as foam.

MS: 604.78 (M+H),

H¹-NMR (CDCl₃): 1.85 (s, 3H, 2′-CH₃), 4.75-5.00 (m, 3H, sugar), 6.07-6.09 (d, 1H, H-3′), 6.81 (s, 1H, H-1′), 7.25-8.20 (m, 15H, benzoyl), 8.60 & 9.08 (s, 2H, purine).

Step 2. Synthesis of 2′-C-methyl-β-D-riboftranosyl-purine-6-carboxamide

1′,2′,3′,5′-tetra-O-benzoyl-2′-C-methyl-6-carbonitrile-purine (105 mg) was dissolved in a mixture water/methanol/hydrogen peroxide (30%) 1:1:0.05 v/v/v (20 mL). The solution was adjusted to pH 9 with NH₄OH. The mixture was gently heated until a clear solution was obtained and then kept at room temperature overnight. The reaction mixture was evaporated and the residue purified by RP HPLC as previously described. Corresponding fractions were evaporated, co-evaporated with water and dried to provide the desired compound with 60% yield.

MS: 310.78 (M+H),

H¹-NMR (DMSO-d6): 0.82 (s, 3H, 2′-CH₃), 3.80-4.16 (m, 4H, sugar), 5.28-5.35 (m, 3H, sugar), 6.17 (s, 1H, H-1′), 8.74 & 8.86 (s, 2H, purine).

### Example 94

Synthesis of 2-(3,4-Dihydroxy-5-hydroxymethyl-3-methyl-tetrahydro-furan-2-yl)-2H-[1,2,4]triazine-3,5-dione (169)

Step 1. Synthesis of 1,2,3,5-Tetra-O-benzoyl-2′-C-methyl β-D-ribofuranose

The title intermediate was prepared as described herein above.

Step 2. Synthesis of 2-(3,4-Dibenzoyl-5-benzoylmethyl-3-methyl-tetrahydro-furan-2-yl)-2H-[1,2,4]triazine-3,5-dione

2H-[1,2,4]Triazine-3,5-dione (Aldrich) (194.5 mg, 1.72 mmol) was dissolved in anhydrous acetonitrile (6mL). BSA (0.85mL, 3.44 mmol) was added via syringe, and reaction was refluxed at 90° C. for 45 minutes. The reaction was then allowed to cool to room temperature. 1,2,3,5-Tetra-O-benzoyl-2′-C-methyl β-D-ribofuranose (500 mg, 0.861 mmol) was dissolved in anhydrous acetonitrile (6mL) and added to the reaction mixture. TMSOTf (0.625 mL, 3.44 mmol) was then added to the reaction drop wise via syringe. The reaction mixture was then refluxed at 90° C. for 2 hours. The mixture was then diluted with EtOAc (200 mL) and washed with 200 mL saturated NaHCO₃ solution. The organic layer was extracted 2× with 100 mL EtOAc and the combined organic fractions were washed with brine and dried over Magnesium sulfate. The reaction was purified via column chromatography on silica gel (2:4:4 EtOAc:DCM:hexane) to yield a white crystalline product (450 mg, 0.79 mmol, 91%).

H¹-NMR (CDCl₃): 8.13 (m, 4H), 8.00 (dd, 2H), 7.63 (dt, 2H), 7.50 (m, 5H), 7.35 (t, 2H), 7.29 (s, 1H), 7.11 (s, 1H), 6.04 (dd, 1H), 4.85 (dd, 1H), 4.76 (m, 1H), 4.54 (dd, 1H), 1.80 (s, 3H).

Step 3. Synthesis of 2-(3,4-Dihydroxy-5-hydroxymethyl-3-methyl-tetrahydro-furan-2-yl)-2H-[1,2,4]triazine-3,5-dione

35 mg of 2-(3,4-Dibenzoyl-5-benzoylmethyl-3-methyl-tetrahydro-furan-2-yl)-2H-[1,2,4]triazine-3,5-dione was dissolved in ammonia saturated methanol (10 mL). The reaction was sealed and stirred for 48 hours. The reaction was concentrated in vacuo to an amorphous solid and then precipitated from methanol and dichloromethane to obtain product (12 mg, 75% yield).

MS 258.12 (M−H),

H¹-NMR (DMSO-d6): 7.55 (s,1H), 5.95 (s, 1H), 5.00 (s, 2H), 4.55 (s, 1H), 3.80 (t, 1H), 3.65 (dd, 2H), 3.45 (dd, 2H), 1.02 (s, 3H)

### Example 95

Synthesis of 5-Hydroxymethyl-3-methyl-2-(6-thiophen-3-yl-purin-9-yl) tetrahydro-furan-3,4-diol (1)

Step 1. Synthesis of 2-(6-Bromo-purin-9-yl)-5-benzoyloxymethyl-3-methyl-tetrahydro-furan-3,4-oxybenzoyl

6-Bromo-9H-purine (Aldrich, 342.3 mg, 1.72 mmol) was dissolved in anhydrous acetonitrile (6 mL). BSA (0.85 mL, 3.44 mmol) was added via syringe, and reaction was refluxed at 90° C. for 45 minutes. The reaction was then allowed to cool to room temperature. 1,2,3,5-Tetra-O-benzoyl-2′-C-methyl β-D-ribofuranose (500 mg, 0.861 mmol) was dissolved in anhydrous acetonitrile (6 mL) and added to the reaction mixture. TMSOTf (0.625 mL, 3.44 mmol) was then added to the reaction drop wise via syringe. The reaction mixture was then refluxed at 90° C. for 3.5 hours. The mixture was then diluted with EtOAc (100 mL) and washed with 100 mL saturated bicarbonate solution. The organic layer was extracted 2× with 100 mL EtoAc and the combined organic fractions were washed with brine and dried over magnesium sulfate. This mixture was then concentrated in vacuo. The reaction was purified via column chromatography on silica gel (loaded on 5% EtoAc in DCM, eluted with 10%EtoAc in DCM) to yield an off white solid (500 mg, 0.76 mmol, 87%).

H¹-NMR (CDC1₃): 8.75 (s, 1H), 8.40 (s, 1H), 8.12 (dd, 2H), 8.06 (dd, 2H), 8.00 (dd, 2H), 7.65-7.35 (m, 10H), 6.82 (s,1H), 6.21 (d, 1H), 4.95 (m, 2H), 4.75 (m, 1H), 1.61 (s, 3H).

Step 2. 5-Benzoyloxymethyl-3methyl-2-(6-thiophene-3-yl-purin-9-yl)-tetrahydro-furan-3,4-oxybenzoyl

In a sealed reaction vessel, the following reagents were added: 2-(6-Bromo-purin-9-yl)-5-benzoyloxymethyl-3-methyl-tetrahydro-furan-3,4-oxybenzoyl from step 1 above, (240 mg, 0.365 mmol), 3-thiophene boronic acid (Aldrich, 71 mg, 0.548 mmol), potassium carbonate (76 mg, 0.548 mmol), Pd(PPh₃)₄ (42.18 mg, 0.0365 mmol). The reagents were then dissolved in anhydrous toluene (9.6 mL) and stirred at 100° C. overnight. The reaction was diluted with EtoAc (100 mL) and washed 2× with saturated sodium bicarbonate solution (200 mL). The combined organic layers were then washed with brine, dried over sodium sulfate, and concentrated in vacuo. The product was purified via column chromatography on silica gel (1:3 EtoAc: Hexane), and the fractions were concentrated to yield a tan oil (220 mg, 0.33 mmol).

Step 3.5-Hydroxymethyl-3-methyl-2-(6-thiophen-3-yl-purin-9-yl)-tetrahydro-furan-3,4-diol

5-Benzoyloxymethyl-3methyl-2-(6-thiophene-3-yl-purin-9-yl)-tetrahydro-furan-3,4-oxybenzoyl, from Step 2 above, (220 mg, 0.33 mmol) was dissolved in ammonia saturated methanol (20 mL) and stirred at room temperature overnight. The reaction was then concentrated in vacuo and purified via HPLC (0% acetonitrile in water to 100% acetonitrile over 20 minutes. Product eludes at 10.5 minutes) to yield a yellow oil (92 mg, 0.26 mmol, 79%).

MS 349.11 (M+H),

H¹-NMR (DMSO-d6): 8.90 (dd, 1H), 8.86 (s, 1H), 8.81 (s, 1H), 8.24 (dd, 1H), 7.45 (m, 1H), 6.17 (s, 1H), 4.53 (d, 1H), 4.18 (d, 2H), 3.98 (dd, 1H), 0.96 (s, 3H).

### Example 96

Synthesis of 5-Hydroxymethyl-3-methyl-2-(6-phenyl-purin-9-yl)-tetrahydro-furan 3,4-diol (170)

Step 1. 5-Benzoyloxymethyl-3-methyl-2-(6-phenyl-purin-9-yl)-tetrahydro-furan-3,4-oxybenzoyl

In a sealed reaction vessel, the following reagents were added: 2-(6-Bromo-purin-9-yl)-5-benzoyloxymethyl-3-methyl-tetrahydro-furan-3,4-oxybenzoyl (prepared as described above) (200 mg, 0.300 mmol), phenyl boronic acid (Aldrich, 54.9 mg, 0.45 mmol), potassium carbonate (63 mg, 0.45 mmol), Pd(PPh₃)₄ (23 mg, 0.02 mmol). The reagents were then dissolved in anhydrous toluene (6 mL) and stirred at 100° C. overnight. The reaction was then diluted with EtoAc (75 mL) and washed 2× with saturated sodium bicarbonate solution (150 mL). The combined organic layers were then washed with brine, dried over sodium sulfate, and concentrated in vacuo. The product was purified via column chromatography on silica gel (1:4 EtoAc: Hexane), and the fractions were concentrated to yield a colorless oil (153 mg, 0.23 mmol).

Step 2. 5-Hydroxymethyl-3-methyl-2-(6-phenyl-purin-9-yl)-tetrahydro-furan-3,4-diol

The product of Step 1 above(153 mg, 0.23 mmol) was dissolved in ammonia saturated methanol (200 mL) and stirred at room temperature overnight. The reaction was then concentrated in vacuo and purified via HPLC (0% acetonitrile in water to 30% acetonitrile over 20 minutes. Product elutes at 15.3 minutes) to yield a colorless oil (61 mg, 0.18 mmol, 78%).

MS 343.15 (M+H),

H¹-NMR (DMSO-d6): 8.93 (s, 1H), 8.68 (m, 2H), 8.60 (s, 1H), 7.52 (m, 3H), 6.23 (s, 1H), 4.47 (d, 1H), 4.15 (dd, 2H), 3.96 (dd, 1H), 0.85 (s, 3H).

### Example 97

Synthesis of 5-Amino-2-(3,4-dihydroxy-5-hydroxymethyl-3-methyl-tetrahydro-furan-2-yl)-2H-8 1,2,4]triazin-3-one (174) and 5-Amino-2-(3,4-dihydroxy-5-hydroxymethyl-3-methyl-tetrahydro-furan-2-yl)-4,5-dihydro-2H-[1,2,4]triazine-3-thione (172)

Step 1. Synthesis of 2-(3,4-dibenzoyloxy-5-benzoyloxymethyl-3-methyl-tetrahydro-furan-2-yl)-5-thioxo-4,5-dihydro-2H-[1,2,4]triazin-3-one

2-(3,4-Dibenzoyl-5-benzoylmethyl-3-methyl-tetrahydro-furan-2-yl)-2H-[1,2,4]triazine-3,5-dione (450 mg, 0.79 mmol) was dissolved in anhydrous toluene (25 mL). Lawesson's reagent was added (161 mg, 0.4 mmol) and the reaction was refluxed at 120° C. for 4 hours. The reaction was then concentrated in vacuo and co-evaporated with dichloromethane, and purified via column chromatography (3:2:3 DCM:EtoAc:hexane) to yield a yellow oil (160 mg, 0.3 mmol).

Step 2. Synthesis of 5-Amino-2-(3,4-dihydroxy-5-hydroxymethyl-3-methyl-tetrahydro-furan-2-yl)-2H-[1,2,4]triazin-3-one

The product from Step 1 above was dissolved in ammonia saturated methanol (25 mL) and stirred at room temperature overnight. The reaction was then concentrated in vacuo and purified via column chromatography (1:9 MeOH:DCM) to yield a white amorphous solid (5.6 mg, 0.02 mmol)

MS 259.12 (M+H),

H¹-NMR (DMSO-d6): 7.49 (s,1H), 6.08 (s, 1H), 3.79 (d, 1H), 3.7 (d,1H), 3.6 (d, 2H), 3.48 (m, 1H), 0.94 (s,3H)

Step 3: Synthesi of 5-Amino-2-(3,4-dihydroxy-5-hydroxymethyl-3-methyl-tetrahydro-furan-2-yl)-4,5-dihydro-2H-[1,2,4]triazine-3-thione:

The title compound was collected as a separate fraction during the purification in Step 2 above.

MS 274.09 (M−H),

H¹-NMR (DMSO-d6):7.73 (s,1H), 5.91 (s, 1H), 3.81 (dd, 1H), 3.7 (d,1H), 3.60 (d, 1H), 3.48 (dd,1H), 1.03 (s,3H)

### Example 98

Synthesis of 1-(3,4-Dihydroxy-5-hydroxymethyl-3-methyl-tetrahydro-furan-2-yl)-4-hydroxy-1H-pyridin-2-one (177)

Step 1. Synthesis of Benzoic acid 4-(2,4-dichloro-benzyloxy)-5-(2,4-dichloro-benzyloxymethyl)-2-(4-hydroxy-2-oxo-2H-pyridin-1-yl)-3-methyl-tetrahydro-furan-3-yl ester

Pyridine-2,4-diol (Aldrich, 148 mg, 1.33 mmol) was dissolved in anhydrous acetonitrile (6 mL). BSA (0.66 mL, 2.67 mmol) was added via syringe, and reaction was refluxed at 90° C. for 45 minutes. The reaction was then allowed to cool to room temperature. 1,2,3,5-Tetra-O-benzoyl-2′-C-methyl β-D-ribofuranose (400 mg, 0.666 mmol) was dissolved in anhydrous acetonitrile (6 mL) and added to the reaction mixture. TMSOTf (0.482 mL, 2.67 mmol) was then added to the reaction drop wise via syringe. The reaction mixture was then refluxed at 90° C. for 3.5 hours. The mixture was then diluted with EtoAc (200 mL) and washed with 200 mL saturated bicarbonate solution. The organic layer was extracted 2× with 200 mL EtoAc and the combined organic fractions were washed with brine and dried over magnesium sulfate. This mixture was then concentrated in vacuo. The reaction was purified via column chromatography on silica gel (1:19 MeOH:DCM) and concentrated in vacuo to yield a colorless oil (312 mg, 0.82 mmol, 70%).

Step 2. Synthesis of 1-[4-(2,4-Dichloro-benzyloxy)-5-(2,4-dichloro-benzyloxymethyl)-3-hydroxy-3-methyl-tetrahydro-furan-2-yl]-4-hydroxy-1H-pyridin-2-one

The product from Step 1 above (312 mg, 0.46 mmol) was dissolved in potassium carbonate saturated methanol (4.6 mL) and stirred at room temperature overnight. The mixture was then diluted with EtoAc (100 mL) and washed with 100 mL saturated bicarbonate solution, then washed with brine and dried over magnesium sulfate. The magnesium sulfate was filtered off and the solution was concentrated in vacuo to a white powder (265 mg, 0.46 mmol, 100%).

MS 677.96 (M−H).

Step 3. Synthesis of 1-(3,4-Dihydroxy-5-hydroxymethyl-3-methyl-tetrahydro-furan-2-yl)-4-hydroxy-1H-pyridin-2-one

The product from Step 2 above (265 mg, 0.46 mmol) was dissolved in DCM (14 mL) and the temperature was reduced to −78° C. Boron trichloride (1.0M in DCM, 4.6 mL, 4.6 mmol) was added to the reaction dropwise. The reaction was stirred at −78° C. for 2h and then warmed to −20° C. overnight. The reaction was quenched with 1:1 MeOH:DCM (20 mL) and stirred at −20° C. for 15 minutes. NH₄OH was used to neutralize the reaction, and it was then concentrated in vacuo to a tanish solid. The product was purified via column chromatography on silica gel (1:4 MeOH;DCM) to yield a white powder (99 mg, 0.385 mmol, 84%).

MS 256.10 (M−H),

H¹-NMR (DMSO-d6): 7.86 (d, 1H), 6.06 (s, 1H), 5.86 (dd, 1H), 5.54 (d, 1H), 5.12 (dd, 2H), 5.00 (s, 1H), 3.78 (m, 2H), 3.64 (dd, 2H), 0.86 (s, 3H).

### Example 99

Synthesis of 2-(2—Chloro-6-methoxy-purin-9-yl)-5-hydroxymethyl-3-methyl-tetrahydro-furan-3,4,diol

Step 1. Synthesis of 2-(2—Chloro-6-methoxy-purin-9-yl)-4-(2,4-dichloro-benzyloxy)-5-(2,4-dichloro-benzyloxymethyl)-3-methyl-tetrahydro-furan-3-ol

To a solution of 1-methyl-3,5-bis-(2,4-dichloro-benzyloxy)-2—C-methyl-β-D-ribofuranose (400 mg, 0.8 mmol), in anhydrous dichloromethane (13 mL) at 0° C. was add HBr (30% by weight in acetic acid, lmL), dropwise. The resulting solution was stirred at 0° C. for 1 hour, then at room temperature for 3 hours, evaporated in vacuo and co-evaporated with anhydrous toluene (3×20 mL). They oily residue was dissolved in anhydrous acetonitrile (15 mL) and added to a solution of the sodium salt of 2,6-Dichloro-9H-purine, prepared by stirring 2,6-Dichloro-9H-purine (455 mg, 2.4 mmol) with sodium hydride (60% in mineral oil, 110 mg) in anhydrous acetonitrile (50 mL) for 4 hours. The combined mixture was stirred for 24 hours, then evaporated to dryness. The residue was diluted with EtoAc (75 mL) and water (75 mL). The aqueous layer was removed and re-extracted with EtoAc (2×50 mL). The combined organic fractions were then washed with brine (100 mL) and dried over magnesium sulfate. The reaction was purified by column chromatography on silica gel (1:1 EtoAc: hexane) yielding an amorphous solid (400 mg, 0.61 mmol)

Step 2. Synthesis of 2-(2-Chloro-6-methoxy-purin-9-yl)-5-hydroxymethyl-3-methyl-tetrahydro-furan-3,4,diol

The product from Step 1 above was dissolved in dichloromethane (16 mL), and reduced in temperature to −78° C. Boron trichloride (1.0M in DCM, 6.1 mL, 6.1 mmol) was added to the reaction dropwise via syringe. The reaction was stirred at −78° C. for 2 h and then warmed to −20° C. overnight. The reaction was quenched with 1:1 MeOH:DCM (30 mL) and stirred at −20° C. for 15 minutes. The solution was neutralized with NH₄OH and concentrated in vacuo to a foam. The product was purified by column chromatography on silica gel (1:9 MeOH: DCM) yielding a white solid (161 mg, 0.48 mmol, 79%).

MS 331.09 (M+H),

H¹-NMR (DMSO-d6): 8.76 (s, 1H), 5.92 (s, 1H), 5.40 (s, 1H), 5.24 (t, 2H), 4.09 (s, 3H), 3.99 (m, 1H), 3.92 (m, 1H), 3.69 (m, 1H), 0.77 (s, 3H).

### Example 100

Synthesis of 7-(3,4-Dihydroxy-5-hydroxymethyl-3-methyl-tetrahydro-furan-2-yl)-4-oxo-4,7-dihydro-3H-pyrrolo[2,3-d]pyrimidine-5-carboxamidine (203)

Step 1. Synthesis of 5-Bromo-7-(3,4-dihydroxy-5-hydroxymethyl-3-methyl-tetrahydro-furan-2-yl)-3,7-dihydro-pyrrolo[2,3-d]pyrimidin-4-one

7-(3,4-Dihydroxy-5-hydroxymethyl-3-methyl-tetrahydro-furan-2-yl)-3,7-dihydro-pyrrolo[2,3-d]pyrimidin-4-one is dissolved in DMF. NBS is added and the reaction is stirred at room temperature. The completed reaction is then concentrated to a solid, dissolved in EtoAc and washed with water. The organic laye is then washed with brine and dried over sodium sulfate. The solution is then concentrated in vacuo to a solid.

Step 2. Synthesis of 7-(3,4-Dihydroxy-5-hydroxymethyl-3-methyl-tetrahydro-furan-2-yl)-4-oxo-4,7-dihydro-3H-pyrrolo[2,3-d]pyrimidine-5-carbonitrile

The product from Step 1 above is combined with Zn(CN)₂, Pd₂(dba)₃, dppf, and Zn powder in DMF. The reaction is refluxed at 120° C. The completed reaction is purified by column chromatography on silica gel to yield the product.

Step 3. Synthesis of 7-(3,4-Dihydroxy-5-hydroxymethyl-3-methyl-tetrahydro-furan-2-yl)-4-oxo-4,7-dihydro-3H-pyrrolo[2,3-d]pyrimidine-5-carboxamidine

The product from Step 2 above is dissolved in saturated HCl in ethanol and allowed stir at room temperature overnight. The reaction is then concentrated to dryness.

Step 4. Synthesis of 7-(3,4-Dihydroxy-5-hydroxymethyl-3-methyl-tetrahydro-furan-2-yl)-4-oxo-4,7-dihydro-3H-pyrrolo[2,3-d]pyrimidine-5-carboxamidine

The product from Step 3 above is dissolved in liquid ammonia and heated in a bomb overnight. The reaction is then concentrated to yield the final product.

### Example 101

Synthesis of 2-(4-Amino-5-furan-2-yl-pyrrolo[2,3-d]pyrimidin-7-yl)-5-hydroxymethyl-tetrahydro-furan-3,4-diol (204)

Step 1. Synthesis of 4—Chloro-5-iodo-7H-pyrrolo[2,3-d]pyrimidine

4-Chloro-7H-pyrrolo[2,3-d]pyrimidine (TCN) is dissolved in DMF. NIS is added, and the reaction is stirred at room temperature for 1 hour. The reaction is then dissolved in EtoAc, washed with brine, and dried over sodium sulfate. The solution is concentrated down to yield an orange solid.

Step 2. Synthesis of 4-Chloro-5-furan-2-yl-7H-pyrrolo[2,3-d]pyrimidine

The product from Step 1 above is dissolved in dioxane, and the following reagents ware added: 2-furan boronic acid (Aldrich), potassium carbonate, and palladium tetrakis. The reaction vessel is sealed and heated at 100° C. overnight. The reaction is filtered through celite and purified via HPLC to yield a yellow solid.

Step 3. Synthesis of 7-[3,4-Bis-(2,4-dichloro-benzyloxy-5-(2,4-dichloro-benzyloxymethyl)-tetrahydro-furan-2-yl]-4-chloro-5-furan-2-yl-7H-pyrrolo[2,3-d]pyrimidine

To a solution of 1-methyl-3,5-bis-(2,4-dichloro-benzyloxy)-2—C-methyl-β-D-ribofuranose in anhydrous dichloromethane at 0° C. is added HBr (30% by weight in acetic acid, 1 mL), dropwise. The resulting solution is stirred at 0° C. for 1 hour, then at room temperature for 3 hours, evaporated in vacuo and co-evaporated with anhydrous toluene. They oily residue is dissolved in anhydrous acetonitrile and added to a solution of the sodium salt of the product from Step 1 above, which is prepared by stirring the same with sodium hydride (60% in mineral oil) in anhydrous acetonitrile for 4 hours. The combined mixture is stirred for 24 hours, then evaporated to dryness. The residue wis diluted with EtoAc and water. The aqueous layer is removed and re-extracted with EtoAc. The combined organic fractions ware then washed with brine and dried over magnesium sulfate. The reaction is purified by column chromatography on silica gel.

Step 4. Synthesis of 2-(4-chloro-5-furan-2-yl-pyrrolo[2,3-d]pyrimidin-7-yl)-5-hydroxymethyl-tetrahydro-furan-3,4-diol

The product from Step 3 above is dissolved in dichloromethane and the temperature reduced to −78° C. Boron trichloride is added to the reaction dropwise. The reaction is stirred at −78° C. for 2 hours, then at −20° C. overnight. The reaction is quenched with 1:1 MeOH:DCM and stirred at −20° C. for 15 minutes. NH₄OH is used to neutralize the reaction, and it is then concentrated in vacuo to a solid. The product is purified via column chromatography on silica gel.

Step 5. Synthesis of 2-(4-Amino-5-furan-2-yl-pyrrolo[2,3-d]pyrimidin-7-yl)-5-hydroxymethyl-tetrahydro-furan-3,4-diol

The product from Step 4 above is dissolved in liquid ammonia and sealed in a bomb. The reaction is stirred at 80° C. overnight. The solution is concentrated to yield the product.

### Example 102

Synthesis of 2-(4-Amino-5-oxazol-2-yl-pyrrolo[2,3-d]pyrimidin-7-yl)-5-hydroxymethyl-tetrahydro-furan-3,4-diol (205)

Step 1. Synthesis of 4-Chloro-5-oxazol-2-yl-7H-pyrrolo[2,3-d]pyrimidine

4-Chloro-5-iodo-7H-pyrrolo[2,3-d]pyrimidine (as prepared above) is dissolved in THF. Palladium tetrakis(triphenylphosphine) and 2-tributylstannanyl-oxazole (Aldrich) are added to the reaction mixture. The reaction vessel is sealed and heated at 100° C. overnight. The compound is purified via column chromatography on silica gel.

Step 2. Synthesis of 7-[3,4-Bis-(2,4-dichloro-benzyloxy-5-(2,4-dichloro-benzyloxymethyl)-tetrahydro-furan-2-yl]-4-chloro-5-oxazol-2-yl-7H-pyrrolo[2,3-d]pyrimidine

To a solution of 1-methyl-3,5-bis-(2,4-dichloro-benzyloxy)-2—C-methyl-β-D-ribofuranose in anhydrous dichloromethane at 0° C. is added HBr (30% by weight in acetic acid, 1 mL), dropwise. The resulting solution is stirred at 0° C. for 1 hour, then at room temperature for 3 hours, evaporated in vacuo and co-evaporated with anhydrous toluene. They oily residue is dissolved in anhydrous acetonitrile and added to a solution of the sodium salt of the product of Step 1 above, prepared by stirring the same with sodium hydride (60% in mineral oil) in anhydrous acetonitrile for 4 hours. The combined mixture is stirred for 24 hours, then evaporated to dryness. The residue is diluted with EtoAc and water. The aqueous layer is removed and re-extracted with EtoAc. The combined organic fractions are then washed with brine and dried over magnesium sulfate. The reaction is purified by column chromatography on silica gel.

Step 3. Synthesis of 2-(4-chloro-5-furan-2-yl-pyrrolo[2,3-d]pyrimidin-7-yl)-5-hydroxymethyl-tetrahydro-oxazol-3,4-diol

The product of Step 2 above is dissolved in dichloromethane and the temperature is reduced to −78° C. Boron trichloride is added to the reaction dropwise. The reaction is stirred at −78° C. for 2 hours, then at −20° C. overnight. The reaction i quenched with 1:1 MeOH:DCM and stirred at −20° C. for 15 minutes. NH₄OH is used to neutralize the reaction, and it is then concentrated in vacuo to a solid. The product is purified via column chromatography on silica gel.

Step 4. Synthesis of 2-(4-Amino-5-furan-2-yl-pyrrolo[2,3-d]pyrimidin-7-yl)-5-hydroxymethyl-tetrahydro-oxazol-3,4-diol

The product of Step 3 is dissolved in liquid ammonia and sealed in a bomb. The reaction is stirred at 80° C. overnight. The solution is concentrated to yield the desired product.

### Example 103

Synthesis of 4-Cyclopropylamino-1-(3,4-dihydroxy-5-hydroxymethyl-3-methyl-tetrahydro-furan-2-yl)-1H-pyrimidin-2-one (206)

Step 1. Synthesis of 1-(3,4-Dibenzoyloxy-5-benzoyloxynmethyl-3-methyl-tetrahydro-furan-2-yl)-1H-pyrimidine-2,4-dione

1H-Pyrimidine-2,4-dione (Aldrich) is dissolved in anhydrous acetonitrile. BSA is added via syringe, and the reaction is refluxed at 90° C. for 45 minutes. The reaction is then allowed to cool to room temperature. 1,2,3,5-Tetra-O-benzoyl-2′-C-methyl β-D-ribofuranose is dissolved in anhydrous acetonitrile and added to the reaction mixture. TMSOTf is then added to the reaction drop wise via syringe. The reaction mixture is then refluxed at 90° C. for 2 hours. The mixture is then diluted with EtoAc and washed with saturated bicarbonate solution. The organic layer is extracted 2× with EtoAc and the combined organic fractions are washed with brine and dried over Magnesium sulfate. The reaction is purified via column chromatography on silica gel to yield the desired product.

Step 2. Synthesis of 1-(3,4-Dibenzoyloxy-5-benzoyloxynethyl-3-methyl-tetrahydro-furan-2-yl)-4-thioxo-3,4-dihydro-1H-pyrimidin-2-one

The product of Step 1 above is dissolved in anhydrous toluene. Lawesson's reagent is added and the reaction is refluxed at 120° C. for 4 hours. The reaction is then concentrated in vacuo and co-evaporated with dichloromethane, and purified via column chromatography to yield the product.

Step 3. Synthesis of 4-Cyclopropylamino-1-(3,4-dibenzoyloxy-5-benzoyloxymethyl-3-methyl-tetrahydro-furan-2-yl)-1H-pyrimidin-2-one

The product of Step 2 above is dissolved in anhydrous ethanol. Cyclopropylamine (Aldrich) is added, and the reaction is refluxed overnight. The reaction is concentrated in vacuo and purified via column chromatography to yield the product.

Step 4. Synthesis of 4-Cyclopropylamino-1-(3,4-dihydroxy-5-hydroxymethyl-3-methyl-tetrahydro-furan-2-yl)-1H-pyrimidin-2-one

The product of Step 3 above is dissolved in ammonia saturated methanol and stirred at room temperature overnight. The reaction is then concentrated in vacuo and purified via column chromatography on silica gel.

### Example 104

Synthesis of 1-(3,4-Dihydroxy-5-hydroxymethyl-3-methyl-tetrahydro-furan-2-yl)-4-hydrazino-3,4-dihydro-1H-pyrimidin-2-one (207

Step 1. Synthesis of 1-(3,4-Dibenzoyloxy-5-benzoyloxymethyl-3-methyl-tetrahydro-furan-2-yl)-4-hydrazino-3 4-dihydro-1H-pyrimidin-2-one

To a solution of 1-(3,4-Dibenzoyloxy-5-benzoyloxymethyl-3-methyl-tetrahydro-furan-2-yl)-4-thioxo-3,4-dihydro-1H-pyrimidin-2-one in water, hydrazine (35 wt. % solution in water) is added. The reaction is refluxed overnight, then concentrated and purified via column chromatography on silica gel.

Step 2. Synthesis 1-(3,4-Dihydroxy-5-hydroxymethyl-3-methyl-tetrahydro-furan-2-yl)-4-hydrazino-3,4-dihydro-1H-pyrimidin-2-one

The product from Step 1 above is dissolved in ammonia saturated methanol and stirred at room temperature overnight. The reaction wis then concentrated in vacuo and purified via column chromatography on silica gel to yield the desired product.

### Example 106

Synthesis of 8-(3,4-Dihydroxy-5-hydroxymethyl-3-methyl-tetrahydro-furan-2-yl)-4,5-dioxo-3,4,5,8-tetrahydro-pyrido[2,3-d]pyrimidine-6-carboxylic acid amide (161)

8-(3,4-Bis-benzoyloxy-5-benzoyloxymethyl-3-methyl-tetrahydro-furan-2-yl)-2-methylsulfanyl-4,5-dioxo-3,4,5,8-tetrahydro-pyrido[2,3-d]pyrimidine-6-carboxylic acid ethyl ester (0.2 g, 0.270 mmol) was taken up in 30 mL ethanol and Raney nickel (0.55 g weighed wet and pre-treated with DI water followed by ethanol was added and the suspension was heated to reflux for 24 hours. An additional 1.8 grams Raney nickel was added (weighted wet and pretreated as above) and the reaction was refluxed for an additional 24 hours. The suspension was filtered hot and the Raney nickel was washed with hot ethanol. The flow-through was concentrated in vacuo and 1 mL DMSO was added to dissolve nucleoside then diluted with saturated ammonia in methanol (30 mLs). The reaction was allowed to stir at room temperature overnight then was concentrated in vacuo and separated on HPLC 0-20% Buffer B over 30min at a flow rate of 10 mLs/min. Buffer A—0.1% triethylammonium acetate in water, Buffer B—0.1% triethylammonium acetate in CH₃CN. Pooled fractions containing nucleoside and evaporated and dried by co-evaporation with absolute ethanol to yield 7 mg (10%) of the desired nucleoside.

MS: 351.16 (M−H).

H¹NMR (DMSO-d6): 0.8 (s, 3H, 2′-CH₃), 3.0-4.0 (m, 4H, sugar), 5.0-5.5 (m, 3H, OH), 6.7 (s, 1H, 1′-H), 7.6 (s, 1H, —Ar), 8.4 (s, 1H, —Ar), 9.0 and 9.2 (s, 2H, NH₂).

### Example 107

Synthesis of 4-Amino-8-(3,4-dihydroxy-5-hydroxymethyl-3-methyl-tetrahydro-furan-2-yl)-2-methylsulfanyl-8H-pyrido [23-d]pyrimidin-7-one (165)

Step 1. Synthesis of 4-Amino-2-methylsulfanyl-8H-pyrido[2,3-d]pyrimidin-7-one.

4-Amino-2-methylsulfanyl-8H-pyrido[2,3-d]pyrimidin-7-one was synthesized as described in G. L Anderson and S. G.Richardson J.Heterocyclic Chem. 1985, 22, 1735-1737.

Step 2. 4-Amino-8[4(2,4dichlorobenzyloxy)-5-(2,4dichlorobenzyloxymethyl)-3-hydroxy-3-methyl-tetrahydro-furan-2-yl]-2-methylsulfanyl-8H-p rido[2,3-d]pyrimidin-7-one

To a solution of 1-methyl-3,5-bis-(2,4-dichloro-benzyloxy)-2-C-methyl-β-D-ribofuranose (0.5 g, 1.0 mmol) in dry methylene chloride (15 mL) cooled to 0° C. was added HBr (30% by weight in acetic acid, 1.25 mL, 6.27 mmol) dropwise. The mixture was allowed to stir at 0° C. for 1 hour then allowed to warm to room temperature and stirred for an additional 2 hours. The resulting translucent brown solution was concentrated in vacuo and co-evaporated with dry toluene (3×15 mL) resulting in a brown oil. The oil was taken up in DMF (8 mL) and added to the sodium salt solution of 4-Amino-2-methylsulfanyl-8H-pyrido[2,3-d]pyrimidin-7-one (generated in situ by stirring the same (0.624 g, 3.0 mmol) in DMF (40 mL) with NaH (60% dispersion in mineral oil, 0.132 g, 3.3 mmol) at room temperhature for 3 hours). The resulting reaction was allowed to stir at room temperature for 24 h then concentrated in vacuo. The crude product was purified by column chromatography on silica gel using 5% methanol in methylene chloride as the eluent. The appropriate fractions were pooled, concentrated in vacuo to give 340 mg (51%) of a yellow oil.

Step 3. Synthesis of 4-Amino-8-(3,4-dihydroxy-5-hydroxymethyl-3-methyl-tetrahydro-furan-2-yl)-2-methylsulfanyl-8H-pyrido[2,3-d]pyrimidin-7-one.

To a solution of the product of step 2 above (0.34 g, 0.506 mmol) in methylene chloride (16 mL) cooled to −78° C. in a dry ice/acetone bath was added BCl₃ (1M in methylene chloride, 5.0 mL, 5.0 mmol) dropwise. The solution was stirred at −78° C. for 1.5 hours, then at −20° C. for 20 hours. The reaction was placed in an ice bath and neutralized with the addition of aqueous ammonia and stirred at room temperature for 10 min. The resulting boron salts were washed with methylene chloride and concentrated in vacuo. The residue was taken up in DMSO (3 mL) and diluted with H₂O (2 mL) and the product isolated on HPLC 15% B isocratic over 30min with flow rate of 10 mL/min. Buffer A—0.1% triethylammonium acetate in water, Buffer B—0.1% triethylammonium acetate in CH₃CN. Pooled fractions containing nucleoside, concentrated in vacuo. The residue was then precipitated with methylene chloride and decanted to give 20 mg (8%) of the desired nucleoside.

MS: 355.12 (M+H).

H¹-NMR (DMSO-d6): 0.9 (m, 3H, 2′-CH₃), 2.5 (m, 3H, —CH3), 3.5-4.2 (m, 4H, sugar), 5.0-5.5 (m, 3H, —OH), 6.3 (d, 1H, —Ar), 7.1 (s, 1H, 1′-H), 7.8 (s, 2H, —NH2), 8.0 (d, 1H, —Ar).

### Example 108

Synthesis of 4-Amino-8-(3,4-dihydroxy-5-hydroxymethyl-3-methyl-tetrahydro-furan-2-yl)-8H-pyrido[2,3-d]pyrimidin-7-one (182)

Step 1. Synthesis of 4-Amino-8-(3,4-dihydroxy-5-hydroxymethyl-3-methyl-tetrahydro-furan-2-yl)-8H-pyrido[2,3-d]pyrimidin-7-one

To a solution of 4-Amino-8-(3,4-dihydroxy-5-hydroxymethyl-3-methyl-tetrahydro-furan-2-yl)-2-methylsulfanyl-8H-pyrido[2,3-d]pyrimidin-7-one (15 mg, 0.042 mmol) in EtOH (20 mL) was added Raney nickel (1.0 g) weighed wet and pre-treated with DI water followed by ethanol, was added and the suspension was heated to reflux for 20 hours. The suspension was filtered hot and the Raney nickel was washed with hot ethanol. The flow-through was concentrated in vacuo. The crude reaction was dissolved in DMSO (2 mL) and diluted with H₂O (3 mLs) and purified on HPLC 13% B isocratic over 30min with flow rate of 10 mL/min. Buffer A—0.1% triethylammonium acetate in water, Buffer B—0.1% triethylammonium acetate in CH₃CN. Pooled fractions containing nucleoside, concentrated in vacuo. The residue was then precipitated with methylene chloride and decanted to give 2.5 mg (15%) of the desired nucleoside.

MS: 309.12 (M+H).

### Example 109

Synthesis of 2-(6-Amino-8-methyl-purin-9-yl)-5-hydroxymethyl-tetrahydro-furan-3,4-diol

Step 1. Synthesis of 8-Methyl-9H-purin-6-ylamine

4,5,6-Triaminopyrimidine sulfate (3.0 g, 13.4 mmol) and acetamide (1.0 g, 16.9 mmol) were added to a 25 mL autoclave bomb and heated to 240° C. for 6 hours. The crude product was then boiled in H₂O for 1 hour and filtered through a small pad of Celite. The flow through was concentrated and purified by HPLC 0-10% Buffer B over 30 min at a flow rate of 10 mLs/min. Buffer A—0.1% triethylammonium acetate in water, Buffer B—0.1% triethylammonium acetate in CH₃CN. Pooled the appropriate fractions and concentrated in vacuo to give 225 mg (11%) of the title compound.

MS: 150.08 (M+H).

Step 2. Synthesis of N,N-Dimethyl-N′-(8-methyl-9H-purin-6-yl)-formamidine

To a suspension of the product in Step 1 above (225 mg, 1.51 mmol) in MeOH (14 mL) and methylene chloride (7 mL) was added N′N′-dimethylformamide dimethyl acetal (0.8 mL, 4.52 mmol) and the mixture heated to reflux for 24 hours. The resuling yellow solution was concentrated in vacuo to a yellow oil. This oil was co-evaporated with methylene chloride (2×15 mL) and held under high vacuum for 2hours. The crude product was used directly in Step 3, without further purification.

Step 3. Synthesis of Benzoyl Protected 2-(6-Amino-8-methyl-purin-9-yl)-5-hydroxymethyl-tetrahydro-furan-3,4-diol (

To a solution of the product of step 2 above (1.51 mmol) in 1,2-dichloroethane (10 mL) was added BSA (0.8 mL, 3.322 mmol) and heated to reflux for 1.5 hours under argon. The solution was allowed to cool slightly and β-D-ribofuranose 1-acetate 2,3,5-tribenzoate (0.691 g, 1.37 mmol) dissolved in 1,2-dichloroethane (10 mL) was added, followed immediately by TMSOTf (1 mL, 5.48 mmol). The reaction was heated to reflux for 24 hours, then an additional 0.5 mL TMSOTf was added, and the reaction was reflux for an additional 48 hours. The reaction was cooled to room temperature, diluted with methylene chloride, washed with saturated NaHCO₃ (1×75 mL). The aqueous layer was back extracted with methylene chloride (2×50 mL) and the combined organic layers were washed with H₂O (1×75 mL), brine (1×70 mL), then dried over Na₂SO₄ and concentrated in vacuo. The crude product was purified by column chromatography on silica gel using 5% methanol in methylene chloride as the eluent. The appropriate fractions were pooled, concentrated in vacuo to give the desired compound.

MS: 649.21 (M+H).

Step 4. Synthesis of 2-(6-Amino-8-methyl-purin-9-yl)-5-hydroxymethyl-tetrahydro-furan-3,4-diol

The compound from Step 3 above was dissolved in 7M ammonia in MeOH (30 mL) and stirred at room temperature for 24 hours. The reaction was concentrated and the residue taken up in DMSO (1 mL) and water (4 mL) and purified by HPLC 0-10% Buffer B over 30 min at a flow rate of 10 mLs/min. Buffer A—0.1% triethylammonium acetate in water, Buffer B—0.1% triethylammonium acetate in CH₃CN. The appropriate fractions were pooled and concentrated in vacuo to give 60 mg (16% from Step 3) of the desired compound.

MS: 282.09 (M+H).

H¹-NMR (CD3OD): 2.6 (s, 3H, —CH3), 3.6-5.0 (m, 5H, sugar), 5.9 (d,1H, 1′-H), 8.1 (s, 1H, —Ar).

### Example 110

Synthesis of 2-(6-Amino-8-methyl-purin-9-yl)-5-hydroxymethyl-3-methyl-tetrahydro-furan-3,4-diol

Step 1. Synthesis of 2,3,5 tribenzoyl protected-2-(6-Amino-8-methyl-purin-9-yl)-5-hydroxymethyl-3-methyl-tetrahydro-furan-3,4-diol

To a solution of N,N-Dimethyl-N′-(8-methyl-9H-purin-6-yl)-formamidine (1.71 mmol) (the crude product of Step 2 in Example 109), in 1,2-dichloroethane (10 mL) was added BSA (1.0 mL, 4.05 mmol) and heated to reflux for 1.5 hours under argon. The solution was allowed to cool slightly and 1,2,3,5-tetra-0-benzoyl-2′-C-methyl β-D-ribofuranose (0.750 g, 1.29 mmol) dissolved in 1,2-dichloroethane (10 mL) was added, followed immediately by TMSOTf (1.5 mL, 8.3 mmol). The reaction was heated to reflux for 24 hours. The reaction was cooled to room temperature, diluted with methylene chloride, washed with saturated NaHCO3 (1×75 mL). The aqueous layer was back extracted with methylene chloride (2×50 mL) and the combined organic layers were washed with H₂O (1×75 mL), brine (1×70 mL), then dried over Na2SO4 and concentrated in vacuo. The crude product was purified by column chromatography on silica gel using 5% methanol in methylene chloride as the eluent. The appropriate fractions were pooled, concentrated in vacuo to give the title compound.

Step 2. 2-(6-Amino-8-methyl-purin-9-yl)-5-hydroxymethyl-3-methyl-tetrahydro-furan-3,4-diol

The compound from Step 1 above was dissolved in 7M ammonia in MeOH (30 mL) and stirred at room temperature for 24 hours. The reaction was concentrated and the residue taken up in DMSO (1 mL) and water (4 mL) and purified by HPLC 0-10% Buffer B over 30min at a flow rate of 10 mLs/min. Buffer A—0.1% triethylammonium acetate in water, Buffer B—0.1% triethylammonium acetate in CH₃CN. The appropriate fractions were pooled and concentrated in vacuo to give 60 mg (16%, from Step 1) of the desired compound.

MS: 296.13 (M+H).

H¹-NMR (CD3OD): 1.05 (s, 3H, —CH3), 2.6 (s, 3H, —CH3), 3.6-4.2 (m, 4H, sugar), 6.1 (s,1H, 1′-H), 8.7 (s, 1H, —Ar).

### Example 111

Synthesis of 2-[6-Amino-8-(N′-methyl-hydrazino)-purin-9-yl]-5-hydroxymethyl-tetrahydro-furan-3,4-diol (185)

To a solution of 8-bromoadenosine (Aldrich, 0.1 g, 0.289 mmol) in DMF was added methyl hydrazine (0.15 mL, 2.89 mmol) and the mixture was heated to 85° C. for 3 hours. The crude product was purified by column chromatography on silica gel using 2.5% methanol in methylene chloride to wash and the product eluded with 20% methanol. The appropriate fractions were pooled, concentrated in vacuo to give 90 mg (100%) of the title compound.

MS: 312.16 (M+H).

H¹NMR (DMSO-d6): 3.05 (s, 3H, —CH3) 3.4-4.2, 4.85 (m, 5H, sugar), 5.0-5.2, 5.9 (m, 3H, —OH), 4.7 (m, 2H, NH), 6.35 (d, 1H, 1′-H), 6.9 (s, 2H, —NH2), 7.95 (s, 1H, —Ar).

### Example 112

Synthesis of 2-(6-Amino-8-methoxy-purin-9-yl)-5-hydroxymethyl-tetrahydro-furan-3,4-diol

To a solution of 8-bromoadenosine (Aldrich, 0.1 g, 0.289 mmol) in MeOH (25 mL) was added sodium methoxide (0.1 g, 1.81 mmol) and the mixture was heated to 85° C. for 2 hours. The reaction was quenched with Dow-X 500 resin (H⁺), filtered and Dow-X washed with MeOH (15 mL) followed by 7M ammonia in methanol (15 mL). The flowthrough was concentrated and purified by column chromatography on silica gel using 20% methanol in methylene as eluent. The appropriate fractions were pooled, concentrated in vacuo to give 81 mg (94%) of the title compounds.

MS: 298.10 (M+H).

H¹-NMR (DMSO-d6): 4.1 (s, 3H, —CH3) 3.4-4.2, 4.85 (m, 5H, sugar), 5.1-5.5 (m, 3H, —OH), 5.7 (d, 1H, 1′-H), 7.0 (s, 2H, —NH2), 8.0{(s, 1H, —Ar).

### Example 113

Synthesis of 7-(3,4-Dihydroxy-5-hydroxymethyl-3-methyl-tetrahydro-furan-2-yl)-3,7-dihydro-pyrrolo[2,3-d]pyrimidin-4-one (188)

To a solution of 2-(4-amino-pyrrolo[2,3-d]pyrimidin-7-yl)-5-hydroxymethyl-3-methyl-tetrahydrofuran-3,4-diol (0.09 g, 0.321 mmol) in NMP (2 mL) and acetonitrile (2 mL) was added chloroacetaldehyde (50% solution in H20, 40.8 μl, 0.321 mmol) and the mixture was heat to 50° C. for 24 hours. The reaction was concentrated in vacuo diluted with H₂O and purified by HPLC 2% Buffer B, isocratic over 30min at a flow rate of 20 mLs/min. Buffer A—0.1% triflouroacetic acid in water, Buffer B—0.1% trifluoroacetic acid in CH₃CN. The appropriate fractions were pooled and concentrated in vacuo to give 53 mg (59%) of the title compound.

MS: 282.10 (M+H).

H¹-NMR (DMSO-d6): 0.65 (s, 3H, 2′-CH3), 3.5-4.0 (m, 4H, sugar), 6.1 (s, 1H, 1′-H), 6.5 (d, 1H, —Ar), 7.5 (d, 1H, —Ar) 7.9 (s, 1H, —Ar), 11.95, (s, 1H, —NH)

### Example 114

Synthesis of 6-Amino-9-(3,4-dihydroxy-5-hydroxymethyl-3-methyl-tetrahydro-furan-2-yl)-7,9-dihydro-purin-8-one (173)

Step 1. Synthesis of Trifluoro-acetic acid 5-[8-bromo-6-(2,2,2-trifluoro-acetylamino)-purin-9-yl]-4-methyl-3,4-bis-(2,2,2-trifluoro-acetoxy)-tetrahydro-furan-2-ylmethyl ester.

To a suspension of 8-bromoadensoine (Aldrich, 1.0 g, 2.89 mmol) in dry methylene chloride (14.5 mL) was added triflouroacetic anhydride (10 mL, 57.8 mmol) and stirred for 4 hours. The clear solution was concentrated in vacuo and co-evaporated with dry methylene chloride (3×15 mL) and foamed to give 2 g (100%) of the desired compound which was used directly without further purification in Step 2.

Step 2. Synthesis of 6-Amino-9-(3,4-dihydroxy-5-hydroxymethyl-3-methyl-tetrahydro-furan-2-yl)-7,9-dihydro-purin-8-one

To a solution of the product of Step 1 above (1.05 g, 1.45 mmol) in dry acetonitrile (in a pre-dryed flask cooled under argon) was added Cul (13.7 mg, 0.0725 mmol), TEA (3.67 mL, 0.4M), Palladium tetrakis (83 mg, 5 mole %), and Trimethylsilyl acetylene (0.4 mL, 2.90 mmol). The mixture was heated to 80° C. for 20 hours, cooled, passed through short bed of celite and concentrated in vacuo to an oil. The crude product was purified by column chromatography on silica gel using 1:1.6:4 ratio of EtOAc:MeOH:CH2C12 as the eluent. The appropriate fractions were pooled, concentrated in vacuo to an oil which was precipitated with alcohol/ether to give 250 mg (61%) of the title compound.

MS: 284.11 (M+H).

H¹-NMR (DMSO-d6): 3.2-4.2, 4.85 (m, 5H, sugar), 5.0-5.3 (m, 3H, —OH), 5.7 (d, 1H, 1′-H), 6.6 (s, 2H, —NH2), 8.0 (s, 1H, —Ar), 10.4 (s, 1H, —NH).

### Example 115

Synthesis of 2-Hydroxymethyl-5-(1,3a,5,6-tetraaza-as-indacen-6-yl)-tetrahydro-furan-3,4-diol (186).

To a solution of Tubercidin (Sigma, 0.03 g, 0.11 3 mmol) in DMF (2 mL) was added chloroacetaldehyde (14 mL, 0.226 mmol) and heated to 50° C. for 20 hours. The reaction was concentrated in vacuo and purified by column chromatography on silica gel using 20% methanol in methylene as eluent. The appropriate fractions were pooled, concentrated in vacuo to give 30 mg (94%) of the title compound.

MS: 291.12 (M+H).

H¹-NMR (CD3OD): 3.7-4.6 (m, 5H, sugar), 6.25 (d, 1H, 1′-H), 6.85 (d, 1H, —Ar), 7.45 (d, 1H, —Ar), 7.6 (d, 1H, —Ar), 7.9 (d, 1H, —Ar), 8.95 (s, 1H, —Ar).

### Example 116

Synthesis of 5-Hydroxymethyl-3-methyl-2-(1,3a,5,6-tetraaza-as-indacen-6-yl)-tetrahydro-furan-3,4-diol (166)

To a solution of 2-(4-amino-pyrrolo[2,3-d]pyrimidin-7-yl)-5-hydroxymethyl-3-methyl-tetrahydrofuran-3,4-diol (0.7 g, 0.25 mmol) in DMF (12 mL) was added chloroacetaldehyde (50% solution in H2O, 35 μl, 0.275 mmol) in 7.0 μl aliquots every 4 hours over the course of 20hour. After the final addition, the mixture was allowed to stir for 2 hours then concentrated in vacuo and purified by column chromatography on silica gel using 20% methanol in methylene as eluent. The appropriate fractions were pooled, concentrated in vacuo to give 71 mg (94%) of the title compound.

MS: 305.11 (M+H).

H¹-NMR (CD3OD): 0.8 (s, 3H, 2′-CH3), 3.7-4.2 (m, 4H, sugar), 6.4 (s, 1H, 1′-H), 6.85 (d, 1H, —Ar), 7.45 (d, 1H, —Ar), 7.7 (d, 1H, —Ar), 7.9 (d, 1H, —Ar), 8.95 (s, 1H, —Ar).

### Example 117

Synthesis of 2-(4-Amino-6-methyl-pyrrolo[2,3-d]pyrimidin-7-yl)-5-hydroxymethyl-tetrahydro-furan-3,4-diol (219

Step 1. Synthesis of 6-Methyl-7H-pyrrolo[2,3-d]pyrimidin-4-ylamine

N′N′-dimethylformamide dimethyl acetal (1 equiv.) is added to 2,6-diamino pyrimidine in DMF and heated to 80° C. The resuting mono protected compound is purified and converted to the hydrazine with NaNO₂, 6 N HCl, 0° C., then SnCl₂-2H₂O. To the hydrazine in EtOH is added acetone and TEA and refluxed. The resulting hydrazone is heated in the presence of PPA to form the desired product.

Step 2. Synthesis of 2-(4-Amino-6-methyl-pyrrolo[2,3-d]pyrimidin-7-yl)-5-hydroxymethyl-tetrahydro-furan-3,4-diol

The title compound is prepared as described in Step 2 and 3 of Example 107 using β-D-1-O-methyl-2,3,5,-tri(2,4-dichlorobenzyl)-ribofuranose and the compound from Step 1 above.

### Example 118

Synthesis of 2-(4-Amino-6-methyl-pyrrolo[2,3-d]pyrimidin-7-yl)-5-hydroxymethyl-3-methyl-tetrahydro-furan-3,4-diol (220)

The product of Step 1 of Example 117 is silylated and condensed with 1-methyl-3,5-bis-(2,4-dichlorobenzyloxy)-2-C-methyl-β-D-ribofuranose as described in Step 2 and 3 of Example 107.

### Example 119

Synthesis of 4-Amino-8-(3 4-dihydroxy-5-hydroxymethyl-3-methyl-tetrahydro-furan-2-yl)-2-methylsulfanyl-7-oxo-7,8-dihydro-pteridine-6-carboxylic acid amide (230)

Step 1. Synthesis of 4-Amino-2-methylsulfanyl-7-oxo-7,8-dihydro-pteridine-6-carboxylic acid ethyl ester

Synthesis of 4-Amino-7-oxo-7,8-dihydro-pteridine-6-carboxylic acid ethyl ester is synthesized as described in M. Ott and W. Pfleiderer Chem. Ber. 1974, 107, 339-361.

Step 2. Synthesis of 4-Amino-8-(3,4-dihydroxy-5-hydroxymethyl-3-methyl-tetrahydro-furan-2-yl)-2-methylsulfanyl-7-oxo-7,8-dihydro-pteridine-6-carboxylic acid amide

The product of Step 1 above is silylated and condensed with 1,2,3,5-Tetra-O-benzoyl-2′-C-methyl β-D-ribofuranose (See Example 26, Steps 2 and 3) to provide for the title compound.

### Example 120

Synthesis of 4-Amino-8-(3,4-dihydroxy-5-hydroxymethyl-3-methyl-tetrahydro-furan-2-yl)-7-oxo-7,8-dihydro-pteridine-6-carboxylic acid amide

4-Amino-8-(3,4-dihydroxy-5-hydroxymethyl-3-methyl-tetrahydro-furan-2-yl)-2-methylsulfanyl-7-oxo-7,8-dihydro-pteridine-6-carboxylic acid amide is treated with Raney nickel (see Example 108, Step 1) to give the title compound.

### Example 121

Synthesis of 4-Amino-8-(3,4-dihydroxy-5-hydroxymethyl-3-methyl-tetrahydro-furan-2-yl)-5-oxo-5,8-dihydro-pyrido[2,3-d]pyrimidine-6-carboxylic acid amide (225)

Step 1. Synthesis of 4-chloro-5-oxo-5,8-dihydro-pyrido[2,3-d]pyrimidine-6-carboxylic acid ethyl ester

2-Methylsulfanyl-4,5-dioxo-3,4,5,8-tetrahydro-pyrido[2,3-d]pyrimidine-6-carboxylic acid ethyl ester is treated with Raney nickel to remove the thiomethyl group. The resulting compound is refluxed in POCl₃.

Step 2. Synthesis of 4-Amino-8-(3,4-dihydroxy-5-hydroxymethyl-3-methyl-tetrahydro-furan-2-yl)-5-oxo-5,8-dihydro-pyrido[2,3-d]pyrimidine-6-carboxylic acid amide

The product of Step 1 above is silylated and condensed with 1,2,3,5-Tetra-O-benzoyl-2′-C-methyl β-D-ribofuranose and treated with liquid ammonia (See Example 26, Steps 2 and 3).

### Example 122

Synthesis of 4-Amino-8-(3,4-dihydroxy-5-hydroxymethyl-3-methyl-tetrahydro-furan-2-yl)-8H-pyrido[2,3-d]pyrimidin-5-one (226)

Step 1. Synthesis of 4-chloro-8H-pyrido[2,3-d]pyrimidin-5-one

4-chloro-5-oxo-5,8-dihydro-pyrido[2,3-d]pyrimidine-6-carboxylic acid ethyl ester is saponified and then decarboxylated by heating in quinoline in the presence of copper to give the title compound.

Step 2. Synthesis of 4-Amino-8-(3,4-dihydroxy-5-hydroxymethyl-3-methyl-tetrahydro-furan-2-yl)-8H-pyrido[2,3-d]pyrimidin-5-one

The product of Step 1 above is silylated and condensed with 1,2,3,5-Tetra-O-benzoyl-2′-C-methyl β-D-ribofuranose and treated with liquid ammonia (See Example 26, Steps 2 and 3).

### Example 123

Synthesis of 2-(2,4-Dichloro-5H-pyrrolo[3,2-d]pyrimidin-7-yl)-5-hydroxymethyl-3-methyl-tetrahydro-furan-3,4-diole (183)

Step 1. Synthesis of 4-(2,4-Dichloro-benzyloxy)-5-(2,4-dichloro-benzyloxymethyl)-2-(4,6-dichloro-imidazo[4,5-c]pyridin-1-yl)-3-methyl-tetrahydro-furan-3-ol. 4,6-Dichloroimidazo[4,5-c]pyridine was synthesized as described in R. J. Rousseau and R. K. Robins, J. Heterocycl. Chem. 1965, 2, 196-201. To a solution of 4,6-dichloroimidazo[4,5-c]pyridine (400 mg, 2.1 mmol) in 30 mL anhydrous acetonitrile under argon was added at room temperature sodium hydride (60%, 93.2 mg, 2.3 mmol). The solution was allowed to stir for 4 h.

To a solution of 1-methyl-3,5-bis-(2,4-dichloro-benzyloxy)-2—C-methyl-β-D-ribofuranose (350.6 mg, 0.7 mmol) in 15 mL anhydrous dichloromethane under argon at 0° C. was added 6 eq. 30% HBr in acetic acid dropwise. The solution was allowed to stir at 0° C. for 1 hr and then at room temperature for 3 h. The solution was then evaporated in vacuo and coevaporated with toluene. The residue was dissolved in 10 mL anhydrous acetonitrile and added to the solution of the sodium salt, prepared above.

The combined mixture was stirred at room temperature for 24 h, and then evaporated to dryness. The residue was dissolved in ethyl acetate, and washed with water. The water was extracted three times with ethyl acetate. The combined organic extracts were washed with brine and dried with anhydrous sodium sulfate. The solvent was removed in vacuo. Column chromatography was used for final purification to give 252 mg (0.386 mmol, 54.65%) of 4-(2,4-Dichloro-benzyloxy)-5-(2,4-dichloro-benzyloxymethyl)-2-(4,6-dichloro-imidazo[4,5-c]pyridin-1-yl)-3-methyl-tetrahydro-furan-3-ol.

Step 2. Synthesis of 2-(2,4-Dichloro-5H-pyrrolo[3,2-d]pyrimidin-7-yl)-5-hydroxymethyl-3-methyl-tetrahydro-furan-3,4-diole

The product from Step 1 above (252 mg, 0.39 mmol) was dissolved in dichloromethane (10 mL) and the temperature was reduced to −78° C. Boron trichloride (1.0M in dichloromethane, 3.9 mL, 3.9 mmol) was added to the reaction dropwise. The reaction was stirred at −78° C. for 2 h and then warmed to −20° C. overnight. The reaction was quenched with 1:1 methanol:dichloromethane (20 mL) and stirred at −20° C. for 15 minutes. NH40H was used to neutralize the reaction, and it was then concentrated in vacuo to furnish solid. The product was purified via column chromatography on silica gel to yield a white compound (60 mg).

MS 334.08, 336.08 (M+H),

H¹-NMR (CD3OD): 8.90 (s, 1H), 7.87 (s, 1H), 5.97 (s, 1H), 4.02-4.07 (m,3H), 3.84-3.89 (m, 1H), 0.88 (s, 3H).

### Example 124

Synthesis of 2-(4-Amino-2-chloro-5H-pyrrolo[32-d]pyrimidin-7-yl)-5-hydroxymethyl-3-methyl-tetrahydro-furan-3,4-diol. (187)

2-(2,4-Dichloro-5H-pyrrolo[3,2-d]pyrimidin-7-yl)-5-hydroxymethyl-3-methyl-tetrahydro-furan-3,4-diole (183) (40 mg) was evaporated in a metal bomb and the bomb cooled to −80° C. (acetone/dry ice bath). Ammonia (5 mL) was condensed from a gas tank, until the exit needle showed splattering and bomb was sealed. The reaction was then heated to 135° C. for 2 days. Evaporation and TLC showed an almost complete reaction. A column (chloroform:methanol 5:1) gave 20 mg of product.

MS 315.08 (M+H),

H —NMR (CD3OD): 8.53 (s, 1H), 6.99 (s, 1H), 5.83 (s, 1H), 5.54 (d, 1H), 4.02-4.09 (m, 3H), 3.84-3.89 (m, 1H), 0.88 (s, 3H).

### Example 125

Synthesis of 2-(4-Amino-5H-pyrrolo[3,2-d]pyrimidin-7-yl)-5-hydroxymethyl-3-methyl-tetrahydro-furan-3,4-diol. (201)

Compound 187(40 mg) was dissolved in a 1:1 mixture of ethyl acetate and methanol and 100 mg of 10% pd/C were added, as well as 2 mL of 1N aq. Sodium hydroxide solution. Hydrogenation at 40 psi for 3 h gave product, which was evaporated and then purified via silica gel column chromatography (2:1 chloroform: methanol) to give 24 mg of pure title compound.

MS 281.11 (M+H),

H¹-NMR (CD3OD): 8.60 (s, 1H), 7.70 (d, 1H), 6.99 (d, 1H), 5.91 (s, 1H), 4.02-4.09 (m, 3H), 3.84-3.89 (m, 1H), 0.88 (s, 3H).

### Example 126

Synthesis of 4-Chloro-7-fluoro-1-(2+-C-methyl-β-D-ribofuranosyl)imidazo[4,5-c]pyridine (213)

Step 1. Synthesis of 2-(4-Chloro-7-fluoro-imidazo[4,5-cl pyridin-1-yl)-⁴-(2,4-dichloro-benzyloxy)-5-(2,4-dichloro-benzyloxymethyl)-3-methyl-tetrahydro-furan-3-ol

4-Chloro-7-fluoroimidazo[4,5-c]pyridine is synthesized as described in M. -C. Liu et al. Nucleosides, Nucleotides & Nucleic Acids 2001, 20(12), 1975-2000.

To a solution of 1-methyl-3,5-bis-(2,4-dichloro-benzyloxy)-2—C-methyl-β-D-ribofuranose in anhydrous dichloromethane at 0° C. is added HBr (30% by weight in acetic acid, 1 mL), dropwise. The resulting solution is stirred at 0° C. for 1 hour, then at room temperature for 3 hours, evaporated in vacuo and co-evaporated with anhydrous toluene. They oily residue is dissolved in anhydrous acetonitrile and added to a solution of the sodium salt of 4-Chloro-7-fluoroimidazo[4,5-c]pyridine, prepared by stirring 4-Chloro-7-fluoroimidazo[4,5-c]pyridine with sodium hydride (60% in mineral oil) in anhydrous acetonitrile for 4 hours. The combined mixture is stirred for 24 hours, then evaporated to dryness. The residue is diluted with ethyl acetate and water. The aqueous layer is removed and re-extracted with ethyl acetate. The combined organic fractions are then washed with brine and dried over magnesium sulfate. The reaction is purified by column chromatography on silica gel to give the title compound.

Step 2. Synthesis of 4-Chloro-7-fluoro-1-(2′-C-methyl-β-D-ribofuranosyl) imidazo[4,5-c]pyridine.

The product of Step 1 above is dissolved in dichloromethane and the temperature is reduced to −78° C. Boron trichloride (1.OM in dichloromethane) is added to the reaction dropwise. The reaction is stirred at −78° C. for 2 h and then warmed to −20° C. overnight. The reaction is quenched with 1:1 methanol:dichloromethane and stirred at −20° C. for 15 minutes. NH₄OH is used to neutralize the reaction, and it is then concentrated in vacuo. The product is purified via column chromatography on silica gel to give the title compound.

### Example 127

Synthesis of 4-Amino-7-fluoro-1-(2′-C-methyl-β-D-ribofuranosyl)imidazo [4,5-c]pyridine.(214)

A suspension of Compound 213 in anhydrous hydrazine is refluxed for 1 h. The reaction mixture is then evaporated in vacuo to dryness and the residue co-evaporated with ethanol and deoxygenated water. The crude intermediate is then dissolved in desoxygenated water, Raney Nickel catalyst is added and the mixture is the refluxed with stirring under hydrogen for 8 h. The reaction mixture is filtered through Celite while hot, and the catalyst is washed with hot water. The filtrate is evaporated to dryness and purified via column chromatography to give the title compound.

### Example 128

Synthesis of 2-(4-Amino-5H-pyrrolo[3,2-d]pyrimidin-7-yl)-5-hydroxymethyl-3-methyl-tetrahydro-furan-3,4-diol (215)

Step 1. 3,4-Bis-(2,4-dichloro-benzyloxy)-5-(2,4-dichloro-benzyloxymethyl)-2-methoxy-3-methyl-tetrahydro-furan

2.3 g of 1-methyl-3,5-bis-(2,4-dichloro-benzyloxy)-2—C-methyl-β-D-ribofuranose is dissolved in 25 mL DMF. To this solution is added NaH and heated to 60° C. After the hydrogen evolution subsides, 2,4-dichlorobenzyl-chloride is added dropwise at 40° C. The mixture is stirred for another 16 h, then 5 mL methanol are added. Column chromotography (9:1 ethyl acetate/hexanes) gave 1.77 g of product.

Step 2. 3,4-Bis-(2,4-dichloro-benzyloxy)-5-(2,4-dichloro-benzyloxymethyl)-3 methyl-dihydro-furan-2-one

The product of Step 1 above (1.42 g) is dissolved in 40 mL dioxane. To this solution is added 40 mL of 4N HCl and it is heated to 100 deg C. After the 16 hr, the solution is brought to pH 11 with NaHCO₃ (sat.) and extracted with EtOAc(3×100 mL). The combined organic fractions are dried with Na₂SO₄ and evaporated. The crude mixture is dissolved in 15 mL dry methylene chloride and 1.466 g (1.6 eq) of Dess Martin periodinane are added. After stirring for a day the mixture is poured into 40 mL sat. NaHCO₃ containing 9 g of NaHSO₃. Extraction with EtOAc (3×100 mL), drying of organic layers and column chromatography (19:1Hex/EtOAc) gave 0.72 g product.

Step 3. N′-(7-Bromo-5H-pyrrolo[3,2-d]pyrimidin-4-yl)-N,N-dimethyl-formamidine

5H-Pyrrolo[3,2-d]pyrimidin-4-ylamine is synthesized as described by Montgomery and Hewson, J. Org. Chem., 1965, 30, 1528-1531. 5H-Pyrrolo[3,2-d]pyrimidin-4-ylamine is dissolved in methylene chloride and cooled to 0° C. To this solution is added via addition funnel bromine in methylene chloride. After reaction is complete as can be seen via TLC, it is extracted with EtOAc, dried with sodium sulfate and purified via column chromatography. The product is dissolved in DMF and 1.2 eq. DMFdimethylacetal are added. The reaction mixture is heated to 80° C. until reaction is completed via TLC,evaporated, and chromatographed to furnish the title compound.

Step 4. 2-(4-Amino-5H-pyrrolo[3,2-d]pyrimidin-7-yl)-5-hydroxymethyl-3-methyl-tetrahydro-furan-3,4-diol

To a solution of the product of Step 3 above in THF is added at −75° C. n-BuLi. After 1 h at −75° C. a solution of lactone the product of Step 2 above in THF is added at −75° C., stirred for 2 h at this temperature and then allowed to warm to 0° C. over the next 3 h. Saturated NaHCO₃ is added and the mixture extracted with ether. The organic layer is dried with brine, dried over MgSO₄ and concentrated. The residue is dried, dissolved in CH₂Cl₂ and triethylsilane and BF₃OEt₂ are added dropwise at −75° C. The reaction mixture is allowed to warm up overnight, quenched with 1N HCl and stirred for 1 h at room temperature. The organic mixture is neutralized with NaOH and extracted with EtOAc. Organic layers are washed with brine, dried over MgSO₄, concentrated and purified via column chromatography. The resulting compound is dissolved in dichloromethane and the temperature is reduced to −78° C. Boron trichloride (1.0M in dichloromethane) is added to the reaction dropwise. The reaction is stirred at −78° C. for 2 h and then warmed to −20° C. overnight. The reaction is quenched with 1:1 methanol:dichloromethane and stirred at −20° C. for 15 minutes. NH₄OH is used to neutralize the reaction, and it is then concentrated in vacuo. The product is stirred in Ammonia in MeOH overnight. The product is purified via column chromatography on silica gel.

### Example 129

Synthesis of 4-Amino -1-(β-D-ribofuranosyl)imidazo[4,5-c]pyridine. (216)

4-Amino-7-fluoro-1-(P-D-ribofuranosyl)imidazo[4,5-c]pyridine (216) is synthesized as described in R R. J. Rousseau, L. B. Townsend, and R. K. Robins, Biochemistry 1966, 5(2), 756-760.

### Example 130

Synthesis of 4-Chloro-7-fluoro-1-(β-D-ribofuranosyl)imidazo[4,5-c]pyridine. (217)

4-Chloro-7-fluoro-1-(β-D-ribofuranosyl)imidazo[4,5-c]pyridine (217) is synthesized as described in M. -C. Liu et al. Nucleosides, Nucleotides & Nucleic Acids 2001, 20(12), 1975-2000.

### Example 131

Synthesis of 4-Amino-7-fluoro-1-(β-D-ribofuranosyl)imidazo[4,5-c]pyridine. (218) 4-Amino-7-fluoro-1-(β-D-ribofuranosyl)imidazo[4,5-c]pyridine (218) is synthesized as described in M. -C. Liu et al. Nucleosides, Nucleotides & Nucleic Acids 2001, 20(12), 1975-2000.

### Example 132

Synthesis of 5-Hydroxymethyl-3-methyl-2-(7-nitro-imidazo[4,5-b]-pyridin-3-yl)-tetrahydro-furan-3,4-diol (168)

Step1. Synthesis of 7—Nitro-3H-imidazo[4,5-b]pyridine 7—Nitro-3H-imidazo[4,5-b]pyridine was synthesized as described in G. Cristalli, P. Franchetti, M. Grifantini, S. Vittori, T. Bordoni and C. Geroni J. Med. Chem. 1987, 30, 1686-1688.

Step2. Synthesis of 2′,3′,5′-Trisbenzoyl protected 5-Hydroxymethyl-3-methyl-2-(7-nitro-imidazo[4,5-b]-pyridin-3-yl)-tetrahydro-furan-3,4-diol

The product of Step 1 above (131.1 mg, 0.8 mmol) was dissolved in 10 mL dry acetonitrile. 0.5 mL (2.0 mmol) of N,O-bis(trimethylsilyl)acetamide was added, and the solution was kept at reflux until clear—approximately 15 min. Next, 1,2,3,5-tetra-O-benzoyl-2′-C-methyl β-D-ribofuranose (ribose X) (290.3 mg, 0.5 mmol) and trimethylsilyl trifluoromethanesulfonate (0.3 mL, 2.0 mmol) was added to solution. The reaction was kept at reflux for 1 h. After this time the reaction was allowed to cool to room temperature and was quenched by the addition of solid sodium bicarbonate (294 mg). The mixture was further diluted with 60 mL saturated sodium bicarbonate. The product was extracted with chloroform. The organic phase was washed with brine, dried with sodium sulfate and evaporated. The product was a greasy, yellow solid which was taken immediately to the next step in crude form.

MS: 645.23 (M+Na).

Step 3. Synthesis of 5-Hydroxymethyl-3-methyl-2-(7-nitro-imidazo[4,5-b]-pyridin-3-yl)-tetrahydro-furan-3,4-diol

Nucleoside the product of Step 2 above was dissolved in 100 mL 7N ammonia in methanol. The reaction mixture was allowed to stand at 3° C. overnight. The next day liquids were removed in vacuo. The resulting crude mixture was purified via column chromatography on silica gel using 10% methanol in chloroform. The fractions containing the title nucleoside were combined and evaporated to get 121.5 mg (49%) of desired nucleoside.

MS: 311.10(M+H).

### Example 133

Synthesis of 2-(7-Amino-imidazo[4,5-b]pyridin-3-yl)-5-hydroxymethyl-3-methyl-tetrahydro-furan-3,4-diol (61)

5-Hydroxymethyl-3-methyl-2-(7-nitro-imidazo[4,5-b]-pyridin-3-yl)-tetrahydro-furan-3,4-diol (47.0 mg, 0.15 mmol) was dissolved in 20 mL methanol. A portion of palladium on carbon (10%) was added to solution and the reaction mixture was placed under 50 psi hydrogen for 0.5 h. The palladium catalyst was filtered off, and the solvent was removed in vacuo. The product was lyophilized from 1,4-dioxane to produce title nucleoside as a white fluffy powder (34.1 mg, 80%):

MS 281.16 (M+H).

### Example 134

Synthesis of 5-Hydroxymethyl-3-methyl-2-(4-nitro-benzoimidazol-1-yl)-tetrahydro-furan-3,4-diol (175)

Step 1. Synthesis of 4-Nitro-1H-benzoimidazole

4_Nitro-1H-benzoimidazole was synthesized as described in Sagi, G, et. al., J. Med Chem., 35, 24, 1992, 4549-4556.

Step2. Synthesis of 2′,3′,5′-Trisbenzoyl protected 5-Hydroxymethyl-3-methyl-2-(4-nitro-benzoimidazol-1-yl)-tetrahydro-furan-3,4-diol

The product from Step 1 above (130.5 mg, 0.8 mmol) was dissolved in 10 mL dry acetonitrile. 0.5 mL (2.0 mmol) of N,O-bis(trimethylsilyl)acetamide was added, and the solution was kept at reflux until clear—approximately 15 min. Next, 1,2,3,5-Tetra-O-benzoyl-2′-C-methyl β-D-ribofuranose (ribose X) (280.6 mg, 0.5 mmol) and trimethylsilyl trifluoromethanesulfonate (0.3 mL, 2.0 mmol) was added to solution. The reaction was kept at reflux for 1 h. After this time the reaction was allowed to cool to room temperature and was quenched by the addition of solid sodium bicarbonate (294 mg). The mixture was further diluted with 60 mL saturated sodium bicarbonate. The product was extracted with chloroform. The organic phase was washed with brine, dried with sodium sulfate and evaporated. The product was a greasy solid which was immediately taken to the next step in crude form.

MS: 680.20 (M+CH₃COO).

Step 3. Synthesis of 5-Hydroxymethyl-3-methyl-2-(4-nitro-benzoimidazol-1-yl)-tetrahydro-furan-3,4-diol

The product of Step 2 above was dissolved in 100 mL 7N ammonia in methanol. The reaction mixture was allowed to stand at 3° C. overnight. The next day liquids were removed in vacuo. The resulting crude mixture was purified via column chromatography on silica gel using 10% methanol in chloroform. The fractions containing the title nucleoside were combined and evaporated to get 120.2 mg (78%) of the title nucleoside.

MS: 368.14 (M+CH₃COO).

### Example 135

Synthesis of 2-(4-Amino-benzoimidazol-1-yl)-5-hydroxymethyl-3-methyl-tetrahydro-furan-3,4-diol (176)

Nucloeside 5-Hydroxymethyl-3-methyl-2-(4-nitro-benzoimidazol-1-yl)-tetrahydro-furan-3,4-diol (59.3 mg, 0.19 mmol) was dissolved in 20 mL methanol. A portion of palladium on carbon (10%) was added to solution and the reaction mixture was placed under 50 psi hydrogen for 0.5 h. The palladium catalyst was filtered off, and the solvent was removed in vacuo. The product was evaporated from anhydrous ethanol 3 times to produce title nucleoside as a white powder (47.5 mg, 89%):

MS 280.15 (M+H).

### Example 136

Synthesis of 2-(4-Amino-pyrrolo[2,3-b]pyridin-1-yl)-5-hydroxymethyl-3-methyl-tetrahydro-furan-3,4-diol (179)

Step 1. Synthesis of 4-Nitro-1H-pyrrolo[2,3-b]pyridine

4—Nitro-1H-pyrrolo[2,3-b]pyridine was synthesized as described in Antonini, I, et. al., J. Med. Chem, 1982, 25, 1261-1264.

Step 2. Synthesis of 4-(2,4-Dichloro-benzyloxy)-5-(2,4-dichloro-benzyloxymethyl)-3-methyl-2-(4-nitro-pyrrolo[2,3-b]pyridin-1-yl)-tetrahydro-furan-3-ol

To a solution of the product of Step 1 above (188.9 mg, 1.2 mmol) in 30 mL anhydrous acetonitrile under argon at room temperature was added sodium hydride. The solution was allowed to stir for 4 h. To a solution of the β-D-1-O-methyl-2,3,5,-tri(2,4-dichlorobenzyl)-ribofuranose (sugar Y) (191.5 mg, 0.39 mmol) in 15 mL anhydrous dichloromethane under argon at 0° C. was added 0.46 mL HBr (30%) dropwise. The resulting solution was allowed to stir at 0° for 1 h and then at room temperature for 3 h. The solution was then evaporated in vacuo and coevaporated with toluene. The residue was dissolved in 10 mL anhydrous acetonitrile and added to the solution of the sodium salt of the product of Step 1 above. The combined mixture was stirred at room temperature for 24 h, and then evaporated to dryness. The residue was dissolved in EtOAc, and washed with water. The water was extracted 3× with EtOAc. The combined organic extracts were washed with brine and dried with Na₂SO₄. The solvent was removed in vacuo. Column chromatography with silica gel using 30% ethyl acetate in hexane was used for final purification. The title nucleoside was isolated as a dark brown oil (102.6 mg, 42%).

MS: 686.04 (M+CH₃COO).

Step 3. Synthesis of 5-Hydroxymethyl-3-methyl-2-(4-nitro-pyrrolo[2,3-b]pyridin-1-yl)-tetrahydro-furan-3,4-diol

The product of Step 2 above (102.6 mg, 0.16 mmol) was dissolved in 10 mL CH₂Cl₂ under argon. The solution was brought to −78° C., and BCl₃ (0.164 mL, 1.6 mmol) was added drop-wise over 5 min. The solution was allowed to stir for 2.5 hr at which time the flask was placed in a −20° C. environment overnight. After 20 h., the reaction flask was allowed to warm to room temperature, and quenched with 10 mL methanol: dichlormethane (1:1 ratio, 0.016M). The reaction flask was placed back in the 20° C. environment for 15 min., and then brought to alkaline conditions with 27% NH₄OH. The neutralized crude was evaporated in vacuo, and the product was isolated via column chromatography on silica gel using 10% methanol in chloroform as the running solvent. 37.0 mg (73%) of the title nucleosidewas isolated.

MS: 310.13 (M+H).

Step 4. Synthesis of 2-(4-Amino-pyrrolo[2,3-b]pyridin-1-yl)-5-hydroxymethyl-3-methyl-tetrahydro-furan-3,4-diol

The product of Step 3 above (24.7 mg, 0.08 mmol) was dissolved in 10 mL ethyl acetate. A portion of palladium on carbon (10%) was added to the mixture, which was placed in a hydrogen atmosphere for 30 min. The palladium catalyst was immediately filtered off, and the solvent was removed in vacuo. The title nucleoside was isolated as a pink solid (20.5 mg, 92%).

MS: 280.13 (M+H).

### Example 137

Synthesis of 2-(4,6-Dichloro-pyrrolo[3,2-c]pyridin-1-yl)-5-hydroxymethyl-3-methyl-tetrahydro-furan-3,4-diol (210)

Step 1. Synthesis of 4 4,6-Dichloro-1H-pyrrolo[3,2-c]pyridine

4,6-Dichloro-1H-pyrrolo[3,2-c]pyridine was synthesized as described in Scneller, S. W., Hosmane, R. S., J. Heterocyclic Chem, 15, 325 (1978).

Step 2. Synthesis of 4-(2,4-Dichloro-benzyloxy)-5-(2,4-dichloro-benzyloxymethyl)-2-(4,6-dichloro-pyrrolo[3,2-c]pyridin-1-yl)-3-methyl-tetrahydro-furan-3-ol

To a solution of the base prepared in step 1 above (1.01 g, 5.4 mmol) in 150 mL anhydrous acetonitrile under argon at room temperature was added sodium hydride (60%, 260 mg, 6.5 mmol). The solution was allowed to stir for 4 h. To a solution of the β-D-1-O-methyl-2,3,5,-tri(2,4-dichlorobenzyl)-ribofuranose (sugar Y) (1.11 g, 2.2 mmol) in 75 mL anhydrous dichloromethane under argon at 0° C. was added 0.86 mL HBr (30%) dropwise. The resulting solution was allowed to stir at 0° for 1 h and then at room temperature for 3 h. The solution was then evaporated in vacuo and coevaporated with toluene. The residue was dissolved in 50 mL anhydrous acetonitrile and added to the solution of the sodium salt of base prepared in Step 1 above. The combined mixture was stirred at room temperature for 24 h, and then evaporated to dryness. The residue was dissolved in EtOAc, and washed with water. The water was extracted 3× with EtOAc. The combined organic extracts were washed with brine and dried with Na₂SO₄. The solvent was removed in vacuo. Column chromatography with silica gel using 30% ethyl acetate in hexane was used for final purification. The title nucleoside was isolated as a dark brown oil (724.3 mg, 51%).

MS: 708.9555 (M+CH3COO).

Step 3. Synthesis of 2-(4,6-Dichloro-pyrrolo[3,2-c]pyridin-1-yl)-5-hydroxymethyl-3-methyl-tetrahydro-furan-3,4-diol

The product of Step 2 above (724.3 mg, 1.11 mmol) was dissolved in 22.5 mL CH₂Cl₂ under argon. The solution was brought to −78° C., and Bl₃ (0.98 mL, 1.6 mmol) was added drop-wise over 5 min. The solution was allowed to stir for 2.5 hr at which time the flask was placed in a −20° C. environment overnight. After 20 h., the reaction flask was allowed to warm to room temperature, and quenched with 70 mL methanol: dichloromethane (1:1 ratio, 0.016M). The reaction flask was placed back in the 20° C. environment for 15 min., and then brought to alkaline conditions with 27% NH₄OH. The neutralized crude was evaporated in vacuo, and the product was isolated via column chromatography on silica gel using 10% methanol in chloroform as the running solvent. 269.5 mg (73%) of the title nucleoside was isolated.

MS: 333.04 (M+H).

### Example 138

Synthesis of 2-(4-Amino-6-chloro-pyrrolo[3,2-c]pyridin-1-yl)-5-hydroxymethyl-3-methyl-tetrahydro-furan-3,4-diol (211)

2-(4,6-Dichloro-pyrrolo[3,2-c]pyridin-1-yl)-5-hydroxymethyl-3-methyl-tetrahydro-furan-3,4-diol (269.5 mg, 0.81 mmol) was placed in a metal reaction bomb and was dissolved in liquid ammonia. The bomb was sealed and the apparatus was immersed in an oil bath at 135° C. for 5 days. After that time, the bomb was cooled to −78° C., unsealed and the liquid ammonia was allowed to evaporate. The crude reaction product was purified via column chromatography on silica gel using 20% methanol in chloroform. The title nucleoside was isolated at 130.0 mg (51%).

### Example 139

Synthesis of 2-(4-Amino-pyrrolo[3,2-c]pyridin-1-yl)-5-hydroxymethyl-3-methyl-tetrahydro-furan-3,4-diol (212)

2-(4-Amino-6-chloro-pyrrolo[3,2-c]pyridin-1-yl)-5-hydroxymethyl-3-methyl-tetrahydro-furan-3,4-diol was dissolved in 20 mL methanol to which a portion of palladium on carbon (10%) and 2 mL sodium hydroxide (1N) was added. The reaction mixture was placed under 40 psi hydrogen for 4 hrs. After which time the palladium catalyst was filtered off and the solvent was removed in vacuo. The reaction mixture was purified via column chromatography on silica gel using 33% methanol in chloroform as the eluting solvent.

### Biological Examples

### Example 1

Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture is disclosed in U.S. Pat. No. 5,738,985 to Miles et al. In vitro assays have been reported in Ferrari et al. Jnl. of Vir., 73:1649-1654, 1999; Ishii et al, Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedom and A. Reinoldus as inventors, which claims priority to U.S. Ser. No. 60/004,383, filed on September 1995, describes an HCV polymerase assay that can be used to evaluate the activity of the of the compounds described herein. Another HCV polymerase assay has been reported by Bartholomeusz, et. al., Hepatitis C Virus (HCV) RNA polymerase assay using cloned HCV non-structural proteins; Antiviral Therapy 1996:1(Supp 4) 18-24.

Screens that measure reductions in kinase activity from HCV drugs are disclosed in U.S. Pat. No. 6,030,785, to Katze et al., U.S. Pat. No. Delvecchio et al., and U.S. Pat. No. 5,759,795 to Jubin et al. Screens that measure the protease inhibiting activity of proposed HCV drugs are disclosed in U.S. Pat. No. 5,861,267 to Su et al., U.S. Pat. No. 5,739,002 to De Francesco et al, and U.S. Pat. No. 5,597,691 to Houghton et al.

### Example 2

Replicon Assay

A cell line, ET (Huh-lucubineo-ET) is used for screening of compounds of the present invention for HCV RNA dependent RNA polymerase. The ET cell line is stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T12801; K1846T) (Krieger at al, 2001 and unpublished). The ET cells are grown in DMEM, supplemented with 10% fetal calf serum, 2 mM Glutamine, Penicillin (100 IU/mL)/Streptomycin (100 ug/mL), 1× nonessential amino acids, and 250 ug/mL G418 (“Geneticin”). They are all available through Life Technologies (Bethesda, Md.). The cells are plated at 0.5-1.0×10⁴ cells/well in the 96 well plates and incubated for 24 hrs before adding nucleoside analogs. Then the compounds each at 5 and 50 uM will be added to the cells. Luciferase activity will be measured 48-72 hours later by adding a lysis buffer and the substrate (Catalog number Glo-lysis buffer E2661 and Bright-Glo leuciferase system E2620 Promega, Madison, Wis.). Cells should not be too confluent during the assay. Percent inhibition of replication will be plotted relative to no compound control. Under the same condition, cytotoxicity of the compounds will be determined using cell proliferation reagent, WST-1(Roche, Germany). The compounds showing antiviral activities, but no significant cytotoxicities will be chosen to determine IC₅₀ and TC₅₀.

### Example 3

Cloning and Expression of Recombinant HCV-NS5b

The coding sequence of NS5b protein is cloned by PCR from pFKI₃₈₉luc/NS3-3′/ET as described by Lohmann, V., et al. (1999) Science 285, 110-113 using the following primers:

aggacatggatccgcggggtcgggcacgagacag (SEQ. ID. NO. 1)

aaggctggcatgcactcaatgtcctacacatggac (SEQ. ID. NO. 2)

The cloned fragment is missing the C terminus 21 amino acid residues. The cloned fragment is inserted into an IPTG-inducible expression plasmid that provides an epitope tag (His)6 at the carboxy terminus of the protein.

The recombinant enzyme is expressed in XL-1 cells and after induction of expression, the protein is purified using affinity chromatography on a nickel-NTA column. Storage condition is 10 mM Tris-HCl pH 7.5, 50 mM NaCl, 0.1 mM EDTA, 1 mM DTT, 20% glycerol at −20° C.

### Example 4

HCV-NS5b Enzyme Assay

The polymerase activity is assayed by measuring incorporation of radiolabeled UTP into a RNA product using a poly-A template (1000-10000 nucleotides) and oligo-U₁₂ primer. Alternatively, a portion of the HCV genome is used as template and radiolabeled GTP is used. Typically, the assay mixture (50 μl) contains 10 mM Tris-HCl (pH7.5), 5 mM MgCl₂, 0.2 mM EDTA, 10 mM KCl, 1 unit/μl RNAsin, 1 mM DTT, 10 μM each of NTP, alpha-[³²P]-GTP, 10 ng/μl polyA template and 1 ng/μl oligou primer. Test compounds are dissolved in water containing 0 to 1% DMSO. Typically, compounds are tested at concentrations between 1 nM and 100 μM. Reactions are started with addition of enzyme and allowed to continue at room temperature or 30° C. for 1 to 2 hours. Reactions are quenched with 20 μl 10 mM EDTA and reaction mixtures (50 μl) spotted on DE81 filter disc to capture the radiolabelled RNA products. After washing with 0.5 mM Na₂HPO₄ (3 times), water (1 time) and ethanol (1 time) to remove unincorporated NTP, the discs are dried and the incorporation of radioactivity is determined by scintillation counting.

### Formulation Examples

The following are representative pharmaceutical formulations containing a compound of Formula Ia, Ib, Ic, IV, IVA, V or VA.

### Example 1

Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Example 2

Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Example 3

Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. to 100 | mL |

### Example 4

Injectable Formulation

The following ingredients are mixed to form an injectable formulation.

| Ingredient | Amount | |
|---|---|---|
| compound of this invention | 0.2 mg-20 | mg |
| sodium acetate buffer solution, 0.4 M | 2.0 | mL |
| HCl (1N) or NaOH (1N) | q.s. to suitable | pH |
| water (distilled, sterile) | q.s. to 20 | mL |

### Example 5

Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Amount |
|---|---|
| compound of the invention | 500 mg |
| Witepsol ® H-15 | balance |

What is claimed is:

1. A compound of Formula Ia, Ib, or Ic 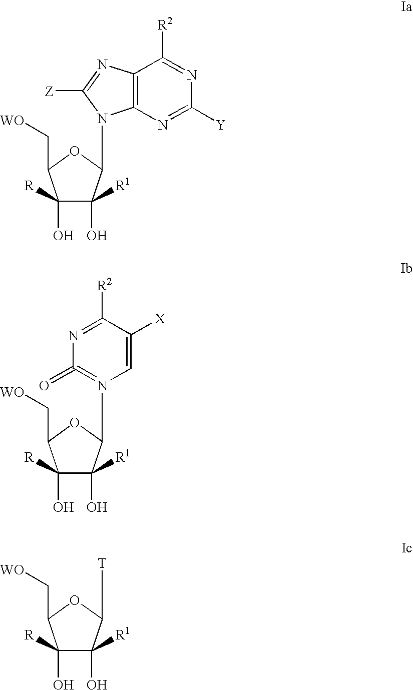
wherein R and R¹ are independently selected from the group consisting of:
hydrogen,
alkyl,
substituted alkyl,
alkenyl,
substituted alkenyl,
alkynyl, and
substituted alkynyl
provided that R and R¹ are not both hydrogen;
R² is selected from the group consisting of:
alkyl,
substituted alkyl,
cycloalkyl,
substituted cycloalkyl,
alkenyl,
substituted alkenyl,
alkynyl,
substituted alkynyl,
acylamino
guanidino
amidino
thioacylamino,
hydroxy,
alkoxy,
substituted alkoxy,
halo,
nitro,
thioalkyl
aryl,
substituted aryl,
heteroaryl,
substituted heteroaryl,
—NR³R⁴ where R³ and R⁴ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic and where R³ and R⁴ are joined to form, together with the nitrogen atom bond thereto, a heterocyclic, substituted heterocyclic, heteroaryl, or substituted heteroaryl,
—NR⁵NR³R⁴ where R³ and R⁴ are as defined above and R⁵ is selected from the group consisting of hydrogen and alkyl,
W is selected from the group consisting of:
hydrogen,
phosphate (including monophosphate, diphosphate, triphosphate or a stablilized phosphate prodrug),
phosphonate,
acyl,
alkyl,
sulfonate ester selected from the group consisting of alkyl esters, substituted alkyl esters, alkenyl esters, substituted alkenyl esters, aryl esters, substituted aryl esters, heteroaryl esters, substituted heteroaryl esters, heterocyclic esters and substituted heterocyclic esters,
a lipid,
an amino acid,
a carbohydrate,
a peptide, and
cholesterol;
X is selected from the group consisting of:
hydrogen,
halo,
alkyl,
substituted alkyl, and
—NR³R⁴ where R³ and R⁴ are as identified above;
Y is selected from the group consisting of:
hydrogen,
halo,
hydroxy,
alkylthio
—NR³R⁴ where R³ and R⁴ are as identified above;
Z is selected from the group consisting of:
hydrogen,
halo,
hydroxy,
alkyl,
azido, and
—NR³R⁴ where R³ and R⁴ are as identified above
—NR⁵NR³R⁴ where R³, R⁴ and R⁵ are as identified above;
and wherein T is selected from the group consisting of
a) 1- and 3-deazapurines of the formula below: 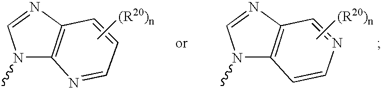
b) purine nucleosides of the formula below: 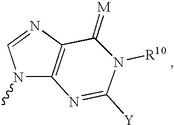
c) benzimidazole nucleosides of the formula below: 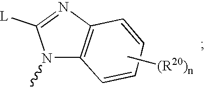
d) 5-pyrrolopyridine nucleosides of the formula below: 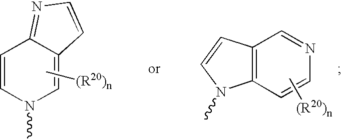
e) 4-pyrimidopyridone sangivamycin analogs of the formula below: 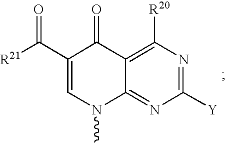
f) 2-pyrimidopyridone sangivamycin analogs of the formula below: 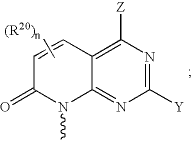
g) 4-pyrimidopyridone sangivamycin analogs of the formula below: 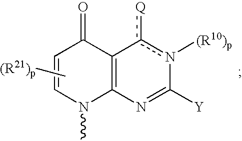
h) pyrimidopyridine analogs of the formulae below: 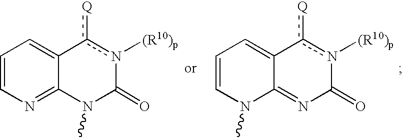
i) pyrimido-tetrahydropyridines of the formula below: 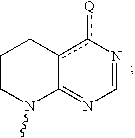
j) Furanopyrimidines (& tetrahydro furanopyrimidines) of the formulae below: 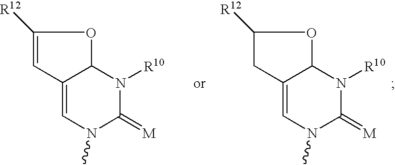
k) pyrazolopyrimidines of the formula below: 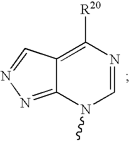
l) pyrolopyrimidines of the formula below: 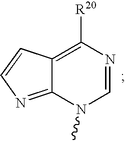
m) triazolopyrimidines of the formula below: 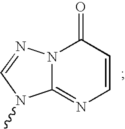
n) pteridines of the formula below: 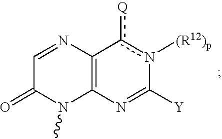
o) pyridine C-nucleosides of the formula below: 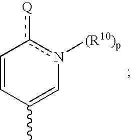
p) pyrazolotriazine C-nucleosides of the formula below: 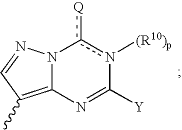
q) Indole nucleosides of the formula below: 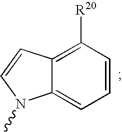
r) a base of the formula below: 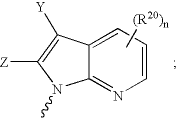
s) a base of the formula below: 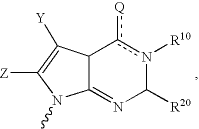
t) a base of the formula below: 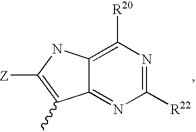
u) a base of the formula below: 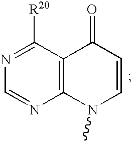
v) a base of the formula below: 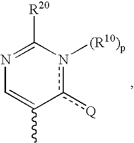
w) a base of the formula below: 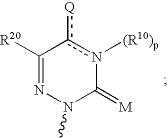
x) a base of the formula below: 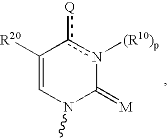
y) a base of the formula below: 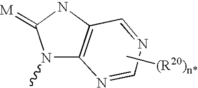
and further wherein one of bonds characterized by [symbol] is a double bond and the other is a single bond provided that, when the [symbol] between the N and a ring carbon is a double bond, then p is 0 and when the [symbol] between Q and a ring carbon is a double bond, then p is 1;
each p is independently 0 or 1;
each n is independently 0 or an integer from 1 to 4;
each n* is independently 0 or an integer from 1 to 2;
L is selected from the group consisting of hydrogen, halo, alkyl, substituted alkyl, amino, substituted amino, azido, and nitro;
Q is selected from the group consisting of hydrogen, halo, ═0,—OR¹¹, ═N—R¹¹, —NHR¹¹, ═S, —SR¹¹, aryl, substituted aryl, heteroaryl, substituted heteroaryl, heterocyclic, and substituted heterocyclic;
M is selected from the group consisting of═O, ═N—R¹¹, and ═S;
Y is as defined above;
R¹⁰ is selected from the group consisting of hydrogen, alkyl, substituted alkyl, cycloalkyl, substituted cycloalkyl, heterocyclic, substituted heterocyclic, alkylthioether, substituted alkylthioether, aryl, substituted aryl, heteroaryl, and substituted heteroaryl, with the proviso that when T is b), s), v), w) or x), then R¹⁰ is not hydrogen;
each R¹¹ and R¹² is independently selected from the group consisting of hydrogen, alkyl, substituted alkyl, cycloalkyl, substituted cycloalkyl, heterocyclic, substituted heterocyclic, amino, substituted amino, alkylthioether, substituted alkylthioether, aryl, substituted aryl, heteroaryl, and substituted heteroaryl;
each R²⁰ is independently selected from the group consisting of:
hydrogen,
alkyl,
substituted alkyl,
aryl,
substituted aryl,
cycloalkyl,
substituted cycloalkyl,
alkenyl,
substituted alkenyl,
alkynyl,
substituted alkynyl,
heteroaryl,
substituted heteroaryl,
acylamino
guanidino
amidino
thioacylamino,
alkoxy,
substituted alkoxy,
alkylthio,
nitro,
halo,
hydroxy
—NR³R⁴ where R³ and R⁴ are as defined above,
—NR⁵NR³R⁴ where R³, R⁴ and R⁵ are as defined above;
each R²¹ and R²² are independently selected from the group consisting of:
—NR³R⁴ where R³ and R⁴ are as defined above, and
—NR⁵NR³R⁴ where R³, R⁴ and R⁵ are as defined above
—C(O)NR³R⁴ where R³ and R⁴ are as defined above, and
—C(O)NR⁵NR³R⁴ where R³, R⁴ and R⁵ are as defined above;
and pharmaceutically acceptable salts thereof;
with the provisos that
1) for a compound of formula Ia, when Z is Z is hydrogen, halo, hydroxy, azido, or NR³R⁴, where R³ and R⁴ are independently H, or alkyl; Y is hydrogen or —NR³R⁴ where R³ and R⁴ are independently hydrogen or alkyl; then R² is not alkyl, alkoxy, halo, hydroxy, CF₃, or —NR³R⁴ where R³ and R⁴ are independently hydrogen or alkyl;
2) for a compound of formula Ia, when Z is hydrogen, halo, hydroxy, azido, or NR³R⁴, where R³ and R⁴ are independently H, or alkyl; Y is hydrogen, halo, hydroxy, or alkylthio; then R is not
alkyl,
substituted alkyl, wherein the substituted alkyl is substituted with hydroxyl, amino, alkylamino, arylamino, alkoxy, aryloxy, nitro, cyano; sulfonic acid, sulfate, phosphonic acid, phosphate, or phosphonate, either unprotected or protected,
halo,
hydroxy,
alkoxy,
thioalkyl, or
—NR³R⁴, where R³ and R⁴ are independently hydrogen, alkyl or alkyl substituted with hydroxyl, amino, alkylamino, arylamino, alkoxy, aryloxy, nitro, cyano, sulfonic acid, sulfate, phosphonic acid, phosphate, or phosphonate, either unprotected or protected);
3) for a compound of formula Ib, when X is hydrogen, halo, alkyl, CF₃ or —NR³R⁴ where R³ is hydrogen and R⁴ is alkyl, then R² is not alkyl, alkoxy, halo, hydroxy, CF₃, or —NR³R⁴ where R³ and R⁴ are independently hydrogen or alkyl;and
4) for a compound of formula Ib, R² is not, halo, alkoxy, hydroxy, thioalkyl, or —NR³R⁴ (where R³ and R⁴ are independently hydrogen, alkyl or alkyl substituted with hydroxyl, amino, alkylamino, arylamino, alkoxy, aryloxy, nitro, cyano, sulfonic acid, sulfate, phosphonic acid, phosphate, or phosphonate, either unprotected or protected) and further with the proviso that the compound of Formual Ia, Ib or Ic is not
c) 2-Hydroxymethyl-5-(6-phenyl-purin-9-yl)-tetrahydro-furan-3,4-diol; or
b) 2-Hydroxymethyl-5-(6-thiophen-3-yl-purin-9-yl)-tetrahydro-furan-3,4-diol.

2. A compound of formula II: 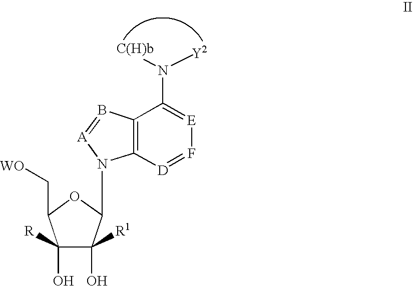
wherein R and R¹ are independently selected from the group consisting of:
hydrogen,
alkyl,
substituted alkyl,
alkenyl,
substituted alkenyl,
alkynyl,
substituted alkynyl,
halogen,
azido,
amino, and
substituted amino
provided that R and R′ are not both hydrogen;
Y² is CH₂, N, S, SO, or SO₂;
N together with —C(H)_{b} and Y² forms a heterocyclic, substituted heterocyclic, heteroaryl or substituted heteroaryl group wherein each of said heterocyclic, substituted heterocyclic, heteroaryl or substituted heteroaryl group is optionally fused to form a bi- or multi-fused ring system (preferably no more than 5 fused rings) with one or more ring structures selected from the group consisting of cycloalkyl, cycloalkenyl, heterocyclic, aryl and heteroaryl group which, in turn, each of such ring structures is optionally substituted with 1 to 4 substituents selected from the group consisting of hydroxyl, halo, alkoxy, substituted alkoxy, thioalkyl, substituted thioalkyl, aryl, heteroaryl, heterocyclic, nitro, cyano, carboxyl, carboxyl esters, alkyl, substituted alkyl, alkenyl, substituted alkenyl, alkynyl, substituted alkynyl, amino, and substituted amino;
b is an integer equal to 0 or 1;
A, B, D, and E are independently selected from the group consisting of >N, >CH, >C—CN, >C—NO₂, >C-alkyl, >C-substituted alkyl, >C—NHCONH₂, >C—CO¹⁵R¹⁶, >C—COOR¹⁵, >C-hydroxy, >C-alkoxy, >C-amino, >C-alkylamino, >C-dialkylamino, >C-halogen, >C-(1,3-oxazol-2-yl), >C-(1,3-thiazol-2-yl) and >C-(imidazol-2-yl);
F is selected from>N, >C—CN, >C—NO₂, >C-alkyl, >C-substituted alkyl, >C—NHCONH₂, >C—CONR¹⁵R¹⁶, >C—COOR¹⁵, >C-alkoxy, >C-(1,3-oxazol-2-yl), >C-(1,3-thiazol-2-yl), >C-(imidazol-2-yl), and >C—Y, where Y is selected from the group consisting of hydrogen, halo, hydroxy, alkylthioether, and —NR³R⁴ where R³ and R⁴ are independently selected from the group consisting of hydrogen, hydroxy, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclic, substituted heterocyclic and where R³ and R⁴ are joined to form, together with the nitrogen atom bond thereto, a heterocyclic group, provided that only one of R³ and R⁴ are hydroxy, alkoxy, or substituted alkoxy;
R¹⁵ and R¹⁶ are independently selected from the group consisting of:
hydrogen,
alkyl,
substituted alkyl,
cycloalkyl,
substituted cycloalkyl,
aryl,
substituted aryl,
heteroaryl,
substituted heteroaryl, and
R¹⁵ and R¹⁶ together with the atom to which they are attached may form a cycloalkyl, substituted cycloalkyl, hetercycloalkyl, substituted heterocylcoalkyl, heteroaryl, or substituted heteroaryl;
W is selected from the group consisting of:
hydrogen,
phosphate (including monophosphate, diphosphate, triphosphate or a stablilized phosphate prodrug),
phosphonate,
acyl,
alkyl,
sulfonate ester selected from the group consisting of alkyl esters, substituted alkyl esters, alkenyl esters, substituted alkenyl esters, aryl esters, substituted aryl esters, heteroaryl esters, substituted heteroaryl esters, heterocyclic esters and substituted heterocyclic esters,
a lipid,
an amino acid,
a carbohydrate,
a peptide, and
cholesterol;
and pharmaceutically acceptable salts thereof.

3. A compound of formula IIA: 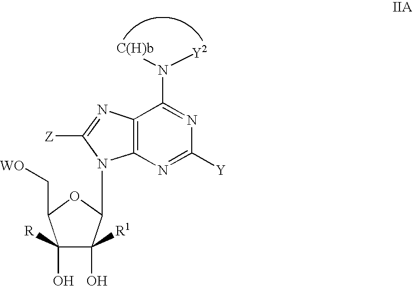
wherein R and R¹ are independently selected from the group consisting of:
hydrogen,
alkyl,
substituted alkyl,
alkenyl,
substituted alkenyl,
alkynyl,
substituted alkynyl,
halogen,
azido,
amino, and
substituted amino;
provided that R and R′ are not both hydrogen;
Y 2is CH₂, N, S, SO, or SO₂;
N together with —C(H)_{b} and Y² forms a heterocyclic, substituted heterocyclic, heteroaryl or substituted heteroaryl group wherein each of said heterocyclic, substituted heterocyclic, heteroaryl or substituted heteroaryl group is optionally fused to form a bi- or multi-fused ring system (preferably no more than 5 fused rings) with one or more ring structures selected from the group consisting of cycloalkyl, cycloalkenyl, heterocyclic, aryl and heteroaryl group which, in turn, each of such ring structures is optionally substituted with 1 to 4 substituents selected from the group consisting of hydroxyl, halo, alkoxy, substituted alkoxy, thioalkyl, substituted thioalkyl, aryl, heteroaryl, heterocyclic, nitro, cyano, carboxyl, carboxyl esters, alkyl, substituted alkyl, alkenyl, substituted alkenyl, alkynyl, substituted alkynyl, amino, and substituted amino;
b is an integer equal to 0 or 1;
W is selected from the group consisting of:
hydrogen,
phosphate (including monophosphate, diphosphate, triphosphate or a stablilized phosphate prodrug),
phosphonate,
acyl,
alkyl,
sulfonate ester selected from the group consisting of alkyl esters, substituted alkyl esters, alkenyl esters, substituted alkenyl esters, aryl esters, substituted aryl esters, heteroaryl esters, substituted heteroaryl esters, heterocyclic esters and substituted heterocyclic esters,
a lipid,
an amino acid,
a carbohydrate,
a peptide, and
cholesterol;
Y is selected from the group consisting of Y is selected from the group consisting of:
hydrogen,
halo,
hydroxy,
alkylthioether
—NR³R⁴ where R³ and R⁴ are independently selected from the group consisting of hydrogen, hydroxy, alkyl, substituted alkyl, alkenyl, substituted alkenyl, alkynyl and substituted alkynyl, alkoxy, substituted alkoxy, aryl, substituted aryl, heteroaryl, substituted heteroaryl, heterocyclic, substituted heterocyclic and where R³ and R⁴ are joined to form, together with the nitrogen atom bond thereto, a heterocyclic group, provided that only one of R³ and R⁴ are hydroxy, alkoxy, or substituted alkoxy;
Z is selected from the group consisting of:
hydrogen,
halo,
hydroxy,
alkyl,
azido, and
—NR³R⁴ where R³ and R⁴ are independently selected from the group consisting of hydrogen, hydroxy, alkyl, substituted alkyl, alkenyl, substituted alkenyl, alkynyl and substituted alkynyl, alkoxy, substituted alkoxy, aryl, substituted aryl, heteroaryl, substituted heteroaryl, heterocyclic, substituted heterocyclic and where R³ and R⁴ are joined to form, together with the nitrogen atom bond thereto, a heterocyclic group, provided that only one of R³ and R⁴ are hydroxy, alkoxy, or substituted alkoxy;
and pharmaceutically acceptable salts thereof.

4. A compound according to any of claims 1-3 wherein R is hydrogen and R¹ is methyl.

5. A compound according to claims 1 and 3 wherein R¹³ and R¹⁴ are hydrogen.

6. A compound according to claims 1 and 3 wherein R¹³ is methyl and R¹⁴ is hydrogen.

7. A compound selected from the group consisting of:
9-(2′-C-methyl-β-D-ribofuranosyl)-6-(thiophen-3-yl)-purine;
9-(2′-C-methyl-β-D-ribofuranosyl)-6-(thiophen-2-yl)-2-aminopurine;
9-(2′-C-methyl-β-D-ribofuranosyl)-6-(pyrrol-3-yl)-purine;
9-(2′-C-methyl-β-D-ribofuranosyl)-6-phenyl-2-aminopurine;
9-(2′-C-methyl-β-D-ribofuranosyl)-6-β-cyanophenyl)-purine;
9-(2′-C-methyl-β-D-ribo furanosyl)-6-(pyridin-3-yl)-purine;
9-(2′-C-methyl-β-D-ribofuranosyl)-6-(Benzo[b]thiophen-3-yl)-2-aminopurine;
9-(2′-C-methyl-β-D-ribofuranosyl)-6-(1H-Indol-5-yl)-purine;
9-(2′-C-methyl-β-D-ribofuranosyl)-6-(naphthalen-2-yl)-purine;
9-(2′-C-methyl-β-D-ribofuranosyl)-6-(dibenzofuran-4-yl)-2-aminopurine;
9-(2′-C-methyl-β-D-ribofuranosyl)-6-(thianthren-1-yl)-purine;
9-(2′-C-methyl-β-D-ribofuranosyl)-6-cyclopropyl-2-aminopurine;
9-(2′-C-methyl-β-D-ribofuranosyl)-6-(ethynyl)-purine;
7-(2′-C-methyl-β-D-ribofuranosyl)-4-thiophen-3-yl-7H-pyrrolo[2,3-d]pyrimidine;
7-(2′-C-methyl-β-D-ribofuranosyl)-4-phenyl-7H-pyrrolo[2,3-d]pyrimidin-2-ylamine;
1-(2′-C-methyl-β-D-ribofuranosyl)-4-thiophen-3-yl-1H-pyrimidin-2-one;
1-(2′-C-methyl-β-D-ribofuranosyl)-4-phenyl-1H-pyrimidin-2-one;
1-(2′-C-Methyl-β-D-ribofuranosyl)-4-benzo[b]thiophen-2-yl-1H-pyrimidin-2-one;
1-(2′-C-methyl-β-D-ribofuranosyl)-;
4-cyclopentyl-1H-pyrimidin-2-one;
9-(2′-C-methyl-β-D-ribofuranosyl)-N⁶-(2-dimethylaminoethyl)-adenine;
9-(2′-C-methyl-β-D-ribofuranosyl)-N⁶-(2-aminoethyl)adenine;
9-(2 ′-C-methyl-β-D-ribofuranosyl)-N⁶-[2-(3H-indol-3-yl)-ethyl]adenine;
9-(2′-C-methyl-β-D-ribofuranosyl)-6-[2-aminocarbonyl-(pyrrolidine-1-yl)]-purine;
1-(2′-C-methyl-β-D-ribofuranosyl)-N⁴-(aminocarbonylmethyl) cytidine;
1-(2′-C-methyl-β-D-ribofuiranosyl)-N⁴-[(pyridin-1-yl)-methyl]cytidine;
9-(2′-C-methyl-β-D-ribofuranosyl)-N⁶-[(adenin-8-yl)-aminoethyl]adenine;
9-(2′-C-methyl-β-D-ribofuranosyl)-N⁶-[(benzene-3,4,5-triol)methyl]adenine;
9-(2′-C-methyl-β-D-ribofuranosyl)-N⁶-[1-aminocarbonyl-2-(3H-indol-3-yl)-ethyl]adenine;
9-(2′-C-methyl-β-D-ribofuranosyl)-6-(1,3,4,9-tetrahydro-beta-carbolin-2-yl)purine;
1-(2′-C-methyl-β-D-ribofuranosyl)-N⁴-[1-aminocarbonyl-2-(3H-indol-3-yl)-ethyl]cytosine;
1-(2′-C-methyl-β-D-ribofuranosyl)-4-(pentafluorophenyl-hydrazino)-pyrimidin-2-one;
1-(2′-C-methyl-β-D-ribofuranosyl)-4-[4-(3,4-dixydroxy-benzyl)-6,7-dihydroxy-3,4-dihydro-1H-isoquinolin-2-yl]-pyrimidin-2-one;
1-(2′-C-methyl-β-D-ribofuranosyl)-N⁴-[2-(3H-indol-3-yl)-ethyl]cytosine;
1-(2′-C-methyl-β-D-ribofuranosyl)-N⁴-(2-aminoethyl)cytosine;
1-(2′-C-methyl-β-D-ribofuranosyl)-N⁴-(aminocarbonyl-isopropyl-methyl)cytidine;
9-(2′-C-methyl-β-D-ribofuranosyl)-N⁶-{[(3H-indol-3-yl)-acetic acid]-hydrazide}adenine;
9-(2′-C-methyl-β-D-ribofuranosyl)-N⁶-[2-(5-fluoro-benzimidazol-1-yl)-ethyl]adenine;
9-(2′-C-methyl-β-D-ribofuranosyl)-6-hydrazino-purine;
9-(2′-C-methyl-β-D-ribofuranosyl)-N⁶-(2,2,3,3,3,-pentafluoropropyl)adenine;
9-(2′-C-methyl-β-D-ribofuranosyl)-6-(piperidin-1-yl)purine;
1-(2′-C-methyl-β-D-ribofuranosyl)-1H-benzimidazole;
3-(2′-C-methyl-β-D-ribofuranosyl)-3H-imidazo[4,5-b]pyridin-7-ylamine;
9-(2 ′-C-trifluoromethyl-β-D-ribofuranosyl)-N⁶-(2-aminoethyl)adenine;
9-(2′-C-trifluoromethyl-β-D-ribofuranosyl)-N⁶-[2-(3H-indol-3-yl)-ethyl]adenine;
9-(2′-C-trifluoromethyl-β-D-ribofuranosyl)-6-[2-aminocarbonyl-(pyrrolidine-1-yl)]-purine;
9-(2′-C-trifluoromethyl-β-D-ribofuranosyl)guanine;
1-(2′-C-trifluoromethyl-β-D-ribofuranosyl)-1H-benzimidazole;
9-(2′-C-ethenyl-β-D-ribofuranosyl)-N⁶-(2-aminoethyl)adenine;
9-(2′-C-ethenyl-β-D-ribofuranosyl)-N⁶-[2-(3H-indol-3-yl)-ethyl]adenine;
9-(2 ′-C-ethenyl-β-D-ribofuranosyl)-6-[2-aminocarbonyl-(pyrrolidine-1-yl)]-purine;
1-(2′-C-ethenyl-β-D-ribofuranosyl)-1H-benzimidazole;
9-(2′-C-ethynyl-β-D-ribofuranosyl)-N⁶-(2-aminoethyl)adenine;
9-(2′-C-ethynyl-β-D-ribofuranosyl)-N⁶-[2-(3H-indol-3-yl)-ethyl]adenine;
9-(2′-C-ethynyl-β-D-ribofuranosyl)-6-[2-aminocarbonyl-(pyrrolidine-1-yl)]-purine;
1-(2′-C-ethynyl-β-D-ribofuranosyl)-1H-benzimidazole;
5-(2′-C-methyl-β-D-ribo furanosyl)-5H-pyrrolo [3,2-c]pyridin-4-ylamine;
4-Amino-8-(2′-C-methyl-β-D-ribofuranosyl)-5-oxo-5,8-dihydro-pyrido[2,3-d]pyrimidine-6-carboxylic acid amide;
2,4-Diamino-8-(2′-C-methyl-β-D-ribofuranosyl)-5-oxo-5,8-dihydro-pyrido[2,3-d]pyrimidine-6-carboxylic acid amide;
4-Amino-8-(2′-C-methyl-β-D-ribofuranosyl)-7-oxo-7,8-dihydro-pyrido[2,3-d]pyrimidine-5-carboxylic acid amide;
2,4-Diamino-8-(2′-C-methyl-β-D-ribofuranosyl)-7-oxo-7,8-dihydro-pyrido[2,3-d]pyrimidine-5-carboxylic acid amide;
8-(2′-C-methyl-β-D-ribofuranosyl)-2-methylsulfanyl-4,5-dioxo-3,4,5,8-tetrahydro-pyrido[2,3-d]pyrimidine-6-carboxylic acid amide;
8-(2′-C-methyl-β-D-ribofuranosyl)-8H-pyrido[2,3-d]pyrimidine-2,4-dione;
1-(2′-C-methyl-β-D-ribofuranosyl)-1H-pyrido [2,3-d]pyrimidine-2,4-dione;
8-(2′-C-methyl-β-D-ribofuranosyl)-4-methylsulfanyl-5,6,7,8-tetrahydro-pyrido[2,3-d]pyrimidine;
3-(2′-C-methyl-β-D-ribofuranosyl)-6-methyl-3,7a-dihydro-1H-furo[2,3-d]pyrimidin-2-one;
3-(2′-C-methyl-β-D-ribofuranosyl)-3,5,6,7a-tetrahydro-1H-furo[2,3-d]pyrimidin-2-one;
7-(2′-C-methyl-β-D-ribofuranosyl)-4-methylsulfanyl-7H-pyrrolo[2,3-d]pyrimidine;
1-(2′-C-methyl-β-D-ribofuranosyl)-4-methylsulfanyl-1H-pyrrolo[2,3-d]pyrimidine;
3-(2′-C-methyl-β-D-ribofuranosyl)-3H-[1,2,4]triazolo[1,5-a]pyrimidin-7-one;
3-methyl-8-(2′-C-methyl-β-D-ribofuranosyl)-2-methylsulfanyl-3H,8H-pteridine-4,7-dione;
5-(2′-C-methyl-β-D-ribofuranosyl)-pyridin-2-ylamine;
5-(2′-C-methyl-β-D-ribofuranosyl)-1H-pyridin-2-one;
8-(2′-C-methyl-β-D-ribofuranosyl)-pyrazolo[1,5-a][1,3,5]triazin-4-ylamine;
8-(2′-C-methyl-β-D-ribofuranosyl)-3H-pyrazolo[1,5-a][1,3,5]triazin-4-one;
2-Amino-8-(2′-C-methyl-β-D-ribofuranosyl)-3H-pyrazolo[1,5-a][1,3,5]triazin-4-one;
1-(2′-C-methyl-β-D-ribofuranosyl)-4-nitroindole;
1-(2′-C-methyl-β-D-ribofuranosyl)-4-aminoindole;
9-(2′-C-methyl-β-D-ribofuranosyl)-6-[2-(1H-imidazol-4-yl)-ethyl]purine;
9-(2′-C-methyl-β-D-ribofuranosyl)-6-(azetidin-1-yl)purine;
9-(2′-C-methyl-β-D-ribofuranosyl)-6-(pyrrolidin-1-yl)purine;
(2′-C-methyl-β-D-ribofuranosyl)-hypoxanthine;
9-(2′-C-methyl-β-D-ribofuranosyl)-6-methylhydrazinopurine;
9-(2′-C-methyl-β-D-ribofuranosyl)-6-(3,6-dihydro-2H-pyridin-1-yl)purine;
9-(2′-C-methyl-β-D-ribofuranosyl)-6-(3,4-dihydro-1H-isoquinolin-2-yl)purine;
2-C-methyl-β-D-ribofuranosyl-6-methythio-purine;
2-C-methyl-β-D-ribofuranosyl-uracil;
2-C-methyl-β-D-ribofuranosyl-thymine;
2-C-methyl-β-D-ribofuranosyl-6-phenyladenin;
9-(2′-C-methyl-β-D-ribofuranosyl)-6-(2-(1H-imidazo-1-4-yl)-ethylamino)purine;
9-(2′-C-methyl-β-D-ribofuranosyl)-6-(2-piperidin-1-yl-ethylamino)purine;
9-(2′-C-methyl-β-D-ribofuranosyl)-6-(cyclopropylamino) purine;
9-(2′-C-methyl-β-D-ribofuranosyl)-6-(cyclopentylamino)purine;
9-(2′-C-methyl-β-D-ribofuranosyl)-6-(cyclohexylamino)purine;
8-(3,4-dihydroxy-5-hydroxymethyl-3-methyl-tetrahydro-furan-2-yl)-4,5-dioxo-3,4,5,8-tetrahydro-pyrido[2,3-d]pyrimidine-6-carboxylic acid amide;
2-(4-Chloro-pyrrolo[2,3-d]pyrimidin-7-yl)-5-hydroxymethyl-3-methyl-tetrahydro-furan-3,4-diol;
9-(2′-C-methyl-β-D-ribofuranosyl)-6-(6-Fluoro-1,3,4,9-tetrahydro-β-carbolin-2-yl)purine;
9-(2′-C-methyl-β-D-ribofuranosyl)-6-(3,6-Dihydro-2H-pyridin-1-yl)purine;
4-Amino-8-(3,4-dihydroxy-5-hydroxymethyl-3-methyl-tetrahydro-furan-2-yl)-2-methylsulfanyl-8H-pyrido[2,3-d]pyrimidin-7-one;
5-Hydroxymethyl-3-methyl-2-(1,3a,5,6-tetraaza-as-indacen-6-yl)-tetrahydro-furan-3,4-diol;
5-Hydroxymethyl-3-methyl-2-(7-nitro-imidazo[4,5-b]-pyridin-3-yl)-tetrahydro-furan-3,4-diol;
2-(3,4-Dihydroxy-5-hydroxymethyl-3-methyl-tetrahydro-furan-2-yl)-;
2H-[1,2,4]triazine-3,5-dione;
5-Hydroxymethyl-3-methyl-2-(6-phenyl-purin-9-yl)-tetrahydro-furan;
3,4-diol;
2-(4-Amino-pyrrolo[2,3-d]pyrimidin-7-yl)-5-hydroxymethyl-3-methyl-tetrahydro-furan-3,4-diol;
5-Amino-2-(3,4-dihydroxy-5-hydroxymethyl-3-methyl-tetrahydro-furan-2-yl)-4,5-dihydro-2H-[1,2,4]triazine-3-thione;
6-Amino-9-(3,4-dihydroxy-5-hydroxymethyl-3-methyl-tetrahydro-furan-2-yl)-7,9-dihydro-purin-8-one;
5-Amino-2-(3,4-dihydroxy-5-hydroxymethyl-3-methyl-tetrahydro-furan-2-yl)-2H-[1,2,4]triazin-3-one;
5-Hydroxymethyl-3-methyl-2-(4-nitro-benzoimidazol-1-yl)-tetrahydro-furan-3,4-diol;
2-(4-Amino-benzoimidazol-1-yl)-5-hydroxymethyl-3-methyl-tetrahydro-furan-3,4-diol;
1-(3,4-Dihydroxy-5-hydroxymethyl-3-methyl-tetrahydro-furan-2-yl)-;
4-hydroxy-1H-pyridin-2-one;
9-(2′-C-methyl-β-D-ribofuranosyl)-6-(tetramethylguanidino)purine;
2-(4-Amino-pyrrolo[2,3-b]pyridin-1-yl)-5-hydroxymethyl-3-methyl-tetrahydro-furan-3,4-diol;
4-Amino-8-(3,4-dihydroxy-5-hydroxymethyl-3-methyl-tetrahydro-furan-2-yl)-8H-pyrido[2,3-d]pyrimidin-7-one;
2-(2,4-Dichloro-5H-pyrrolo[3,2-d]pyrimidin-7-yl)-5-hydroxymethyl-3-methyl-tetrahydro-furan-3,4-diole;
1-(2′-C-methyl-β-D-ribofuranosyl)-5-aminobenzimidazole;
and
1-(2′-C-methyl-β-D-ribofuranosyl)-6-aminobenzimidazole;
2-[6-Amino-8-(N′-methyl-hydrazino)-purin-9-yl]-5-hydroxymethyl-tetrahydro-furan-3,4-diol;
2-Hydroxymethyl-5-(1,3a,5,6-tetraaza-as-indacen-6-yl)-tetrahydro-furan-3,4-diol;
7-(3,4-Dihydroxy-5-hydroxymethyl-3-methyl-tetrahydro-furan-2-yl)-3,7-dihydro-pyrrolo[2,3-d]pyrimidin-4-one;
2-(4-Amino-2-[1,2,4]triazol-1-yl-pyrimidin-5-yl)-5-hydroxymethyl-tetrahydro-furan-3,4-diol;
2-Hydroxymethyl-5-(4-methylamino-2-[1,2,4]triazol-1-yl-pyrimidin-5-yl)-tetrahydro-furan-3,4-diol;
2-Hydroxymethyl-5-[4-methylamino-2-(N′-methyl-hydrazino)-pyrimidin-5-yl]-tetrahydro-furan-3,4-diol;
2-(4-Amino-5H-pyrrolo[3,2-d]pyrimidin-7-yl)-5-hydroxymethyl-3-methyl-tetrahydro-furan-3,4-diol;
7-(3,4-Dihydroxy-5-hydroxymethyl-3-methyl-tetrahydro-furan-2-yl)-;
4-oxo-4,7-dihydro-3H-pyrrolo[2,3-d]pyrimidine-5-carboxamidine;
2-(4-Amino-5-furan-2-yl-pyrrolo[2,3-d]pyrimidin-7-yl)-;
5-hydroxymethyl-tetrahydro-furan-3,4-diol;
2-(4-Amino-5-oxazol-2-yl-pyrrolo[2,3-d]pyrimidin-7-yl)-;
5-hydroxymethyl-tetrahydro-furan-3,4-diol;
4-Cyclopropylamino-1-(3,4-dihydroxy-5-hydroxymethyl-3-methyl-tetrahydro-furan-2-yl)-1H-pyrimidin-2-one;
1-(3,4-Dihydroxy-5-hydroxymethyl-3-methyl-tetrahydro-furan-2-yl)-;
4-hydrazino-3,4-dihydro-1H-pyrimidin-2-one;
2-C-methyl-β-D-ribofuranosyl-purine-6-carboxamide;
9-(3,4-Dihydroxy-5-hydroxymethyl-3-methyl-tetrahydro-furan-2-yl)-9H-purine-6-carbothioic acid amide;
2-(4,6-Dichloro-pyrrolo[3,2-c]pyridin-1-yl)-5-hydroxymethyl-3-methyl-tetrahydro-furan-3,4-diol;
2-(4-Amino-6-chloro-pyrrolo[3,2-c]pyridin-1-yl)-5-hydroxymethyl-3-methyl-tetrahydro-furan-3,4-diol;
2-(4-Amino-pyrrolo[3,2-c]pyridin-1-yl)-5-hydroxymethyl-3-methyl-tetrahydro-furan-3,4-diol;
4-Chloro-7-fluoro-1-(2′-C-methyl-β-D-ribofuranosyl)imidazo[4,5-c]pyridine;
4-Amino-7-fluoro-1-(2′-C-methyl-β-D-ribofuranosyl)imidazo;
[4,5-c]pyridine;
2-(4-Amino-5H-pyrrolo[3,2-d]pyrimidin-7-yl)-5-hydroxymethyl-3-methyl-tetrahydro-furan-3,4-diol;
4-Amino-1-(β-D-ribofuranosyl)imidazo[4,5-c]pyridine;
4-Chloro-7-fluoro-1-(β-D-ribofuranosyl)imidazo[4,5-c]pyridine;
4-Amino-7-fluoro-1-(β-D-ribofuranosyl)imidazo[4,5-c]pyridine;
2-(4-Amino-6-methyl-pyrrolo[2,3-d]pyrimidin-7-yl)-5-hydroxymethyl-tetrahydro-furan-3,4-diol;
2-(4-Amino-6-methyl-pyrrolo[2,3-d]pyrimidin-7-yl)-5-hydroxymethyl-3-methyl-tetrahydro-furan-3,4-diol;
4-Amino-8-(3,4-dihydroxy-5-hydroxymethyl-tetrahydro-furan-2-yl)-7-oxo-7,8-dihydro-pteridine-6-carboxylic acid amide;
4-Amino-8-(3,4-dihydroxy-5-hydroxymethyl-3-methyl-tetrahydro-furan-2-yl)-7-oxo-7,8-dihydro-pteridine-6-carboxylic acid amide;
4-Amino-8-(3,4-dihydroxy-5-hydroxymethyl-3-methyl-tetrahydro-furan-2-yl)-5-oxo-5,8-dihydro-pyrido[2,3-d]pyrimidine-6-carboxylic acid amide;
4-Amino-8-(3,4-dihydroxy-5-hydroxymethyl-3-methyl-tetrahydro-furan-2-yl)-5-oxo-5,8-dihydro-pyrido[2,3-d]pyrimidine-6-carboxylic acid amide;
4-Amino-8-(3,4-dihydroxy-5-hydroxymethyl-tetrahydro-;
furan-2-yl)-5-oxo-5,8-dihydro-pyrido[2,3-d]pyrimidine-6-carboxylic acid amide;
4-Amino-8-(3,4-dihydroxy-5-hydroxymethyl-3-methyl-tetrahydro-furan-2-yl)-8H-pyrido[2,3-d]pyrimidin-5-one;
4-Amino-8-(3,4-dihydroxy-5-hydroxymethyl-tetrahydro-furan-²-yl)-8H-pteridin-7-one;
4-Amino-8-(3,4-dihydroxy-5-hydroxymethyl-tetrahydro-furan-²-yl)-8H-pyrido[2,3-d]pyrimidin-7-one;
4-Amino-8-(3,4-dihydroxy-5-hydroxymethyl-tetrahydro-furan-²-yl)-2-methylsulfanyl-8H-pyrido[2,3-d]pyrimidin-7-one;
of 4-Amino-8-(3,4-dihydroxy-5-hydroxymethyl-3-methyl-tetrahydro-;
furan-2-yl)-2-methylsulfanyl-7-oxo-7,8-dihydro-pteridine-6-carboxylic acid amide;

8. A pharmaceutical composition comprising a pharmaceutically acceptable diluent and a therapeutically effective amount of a compound or mixture of any one of the compounds of claims 1-4 or 6-8.

9. A pharmaceutical composition comprising a pharmaceutically acceptable diluent and a therapeutically effective amount of a compound or mixture of claim 5.

10. A method for treating HCV in mammals which method comprises administering to a mammal diagnosed with HCV or at risk of developing HCV a pharmaceutical composition comprising a pharmaceutical composition of claim 9.
